(19) United States
(12) Patent Application Publication
(10) Pub. No.: US 2006/0241015 A1
(43) Pub. Date: Oct. 26, 2006

(54) Cancer genes

(76) Inventors: Zairen Sun, Rockville, MD (US); Xuan Li, Silver Spring, MD (US); Gilbert Jay, North Bethesda, MD (US); Karl Kovacs, Rockville, MD (US); Wufang Fan, San Diego, CA (US); Youmin Shu, Potomac, MD (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/502,394

(22) PCT Filed: Jan. 24, 2003
(86) PCT No.: PCT/US03/01943

§ 371 Date: Oct. 19, 2005

Related U.S. Application Data
(60) Provisional application No. 60/356,130, filed on Feb. 14, 2002.

(30) Foreign Application Priority Data
Jan. 25, 2002 (US) 10/054935
Mar. 22, 2002 (US) 10/102946
Apr. 8, 2002 (US) 10/117229
May 14, 2002 (US) 10/144198
Jul. 19, 2002 (US) 10/197824

(51) Int. Cl. A61K 38/17; G06Q 10/00; C12Q 1/68; G01N 33/574; C07H 21/04; C12P 21/06; C07K 14/82

(52) U.S. Cl. 514/2; 530/350; 435/69.1; 435/320.1; 435/325; 435/6; 435/7.23; 536/23.5; 705/2

(57) ABSTRACT

The present invention relates to all facets of novel polynucleotides, the polypeptides they encode, antibodies and specific binding partners thereto, and their applications to research, diagnosis, drug discovery, therapy, clinical medicine, forensic science and medicine, etc. The polynucleotides are differentially expressed in prostate and breast cancers and are therefore useful in variety of ways, including, but not limited to, as molecular markers, as drug targets, and for detecting, diagnosing, staging, monitoring, prognosticating, preventing or treating, determining predisposition to, etc., cancers.

This application claims the benefit of U.S. application Ser. No. 10/054,935, filed Jan. 25, 2002; 60/356,130, filed Feb. 14, 2002; Ser. No. 10/102,946, filed Mar. 22, 2002; Ser. No. 10/117,229, filed Apr. 8, 2002; U.S. Ser. No. 10/144,198, May, 14, 2002; Ser. No. 10/197,824, Jul. 19, 2002, which are hereby incorporated by reference in their entirety.

### DESCRIPTION OF THE DRAWINGS

FIGS. 1-18 show amino acid sequence alignments between polypeptides of the present invention, and polypeptides listed in public databases. SEQ ID NOS for the polypeptides of the present invention are listed in Tables 3 and 4. Others are as follows: KIAA0803 (SEQ ID NO 31); KIAA0408 (SEQ ID NO 32); NM_{—}030817 (SEQ ID NO 33); NM_{—}015384 (SEQ ID NO 34); NM_{—}133433 (SEQ ID NO 35); XM_{—}033473 (SEQ ID NO 36); XM_{—}059862 (SEQ ID NO 37); NM_{—}012062 (SEQ ID NO 38); NM_{—}012063 (SEQ ID NO 39); NM_{—}005690 (SEQ ID NO 40); XM_{—}042775 (SEQ ID NO 41); NM_{—}000125 (SEQ ID NO 42); XM_{—}094949 (SEQ ID NO 43); XM_{—}050424 (SEQ ID NO>4; KIAA0534 (SEQ ID NO 76); KIAA1217 (SEQ ID NO 77); KIAA0301 (SEQ ID NO 78); AF441770 (SEQ ID NO 79); XM_{—}085817 (SEQ ID NO 80); AK001276 (SEQ ID NO 81); XM_{—}033473 (SEQ ID NO 82); AK022207 (SEQ ID NO 83).

FIG. 19 shows differential display patterns for genes of the present invention. The white arrowhead indicates the position of a DNA fragment derived from a differentially regulated gene of the present invention. The experiments were performed in duplicate. Each sample set (4 lanes) contains a duplicate from normal (left) prostate tissue and a duplicate tumor (right) prostate tissue from the same individual. There are several sample sets for each gene.

FIG. 20 (A-G) shows the amino acid alignments of human kidins2220 variants (XM_{—}045362, SEQ ID NO 90; and AB033076, SEQ ID NO 91) and rat variants (AF239045, SEQ ID NO 94; and AF313464, SEQ ID NO 93). The referenced numbers are GenBank identifiers.

FIG. 21 shows amino acid alignments between Urb-ctf (—BCU1041,” SEQ ID NO 96), AK014463 (mouse, SEQ ID NO 98) and XM_{—}058887 (human, SEQ ID NO 97). Regions of sequence identity are shaded.

FIG. 22 is the alignment of the amino acid sequences of human BCU399 (SEQ ID NO 100), human XM_{—}059670 (SEQ ID NO 101), a partial sequence for BCU399, and monkey AB071059 (SEQ ID NO 104).

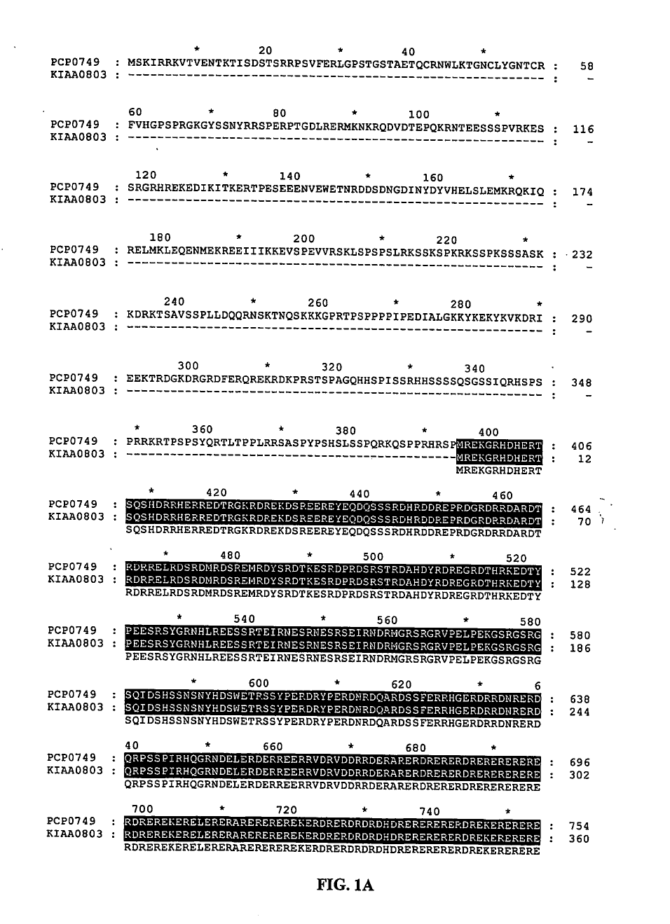

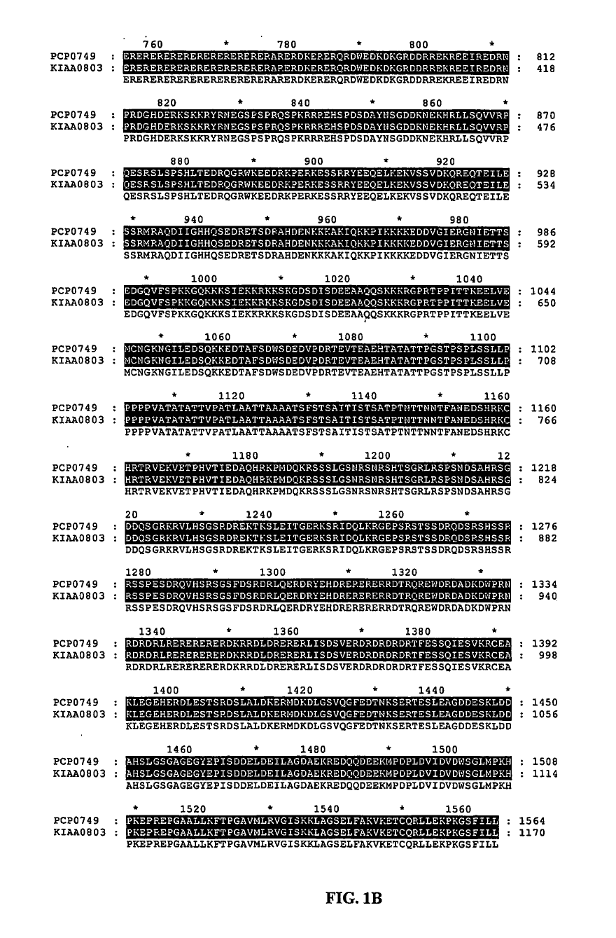

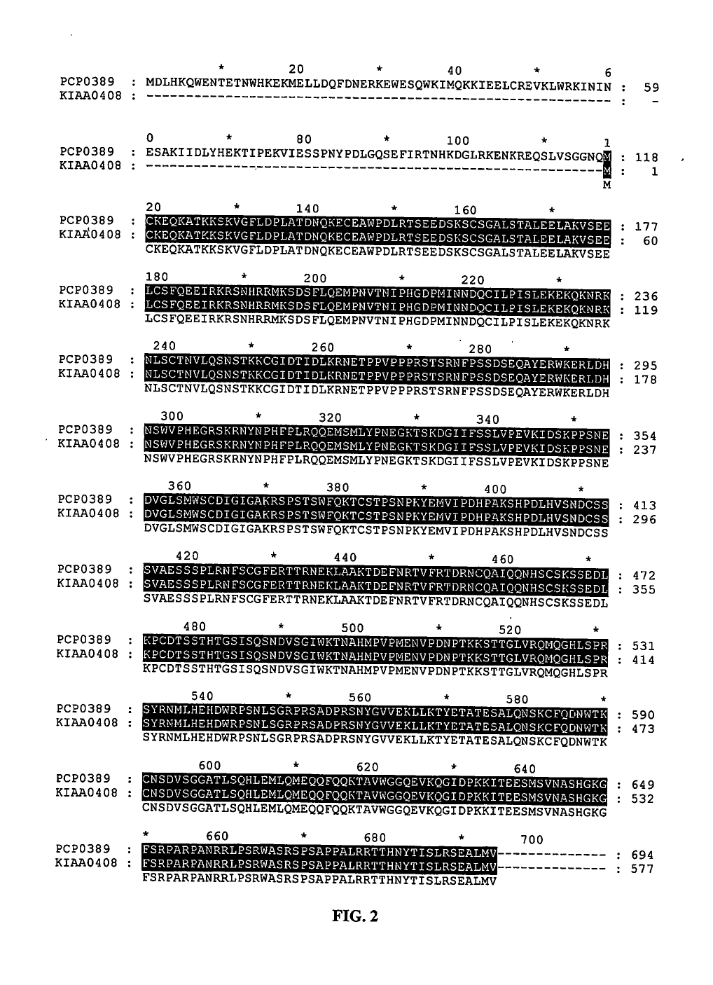

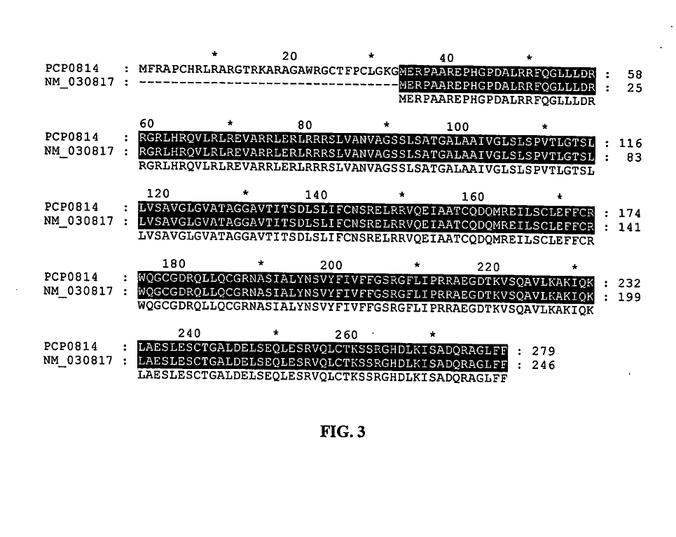

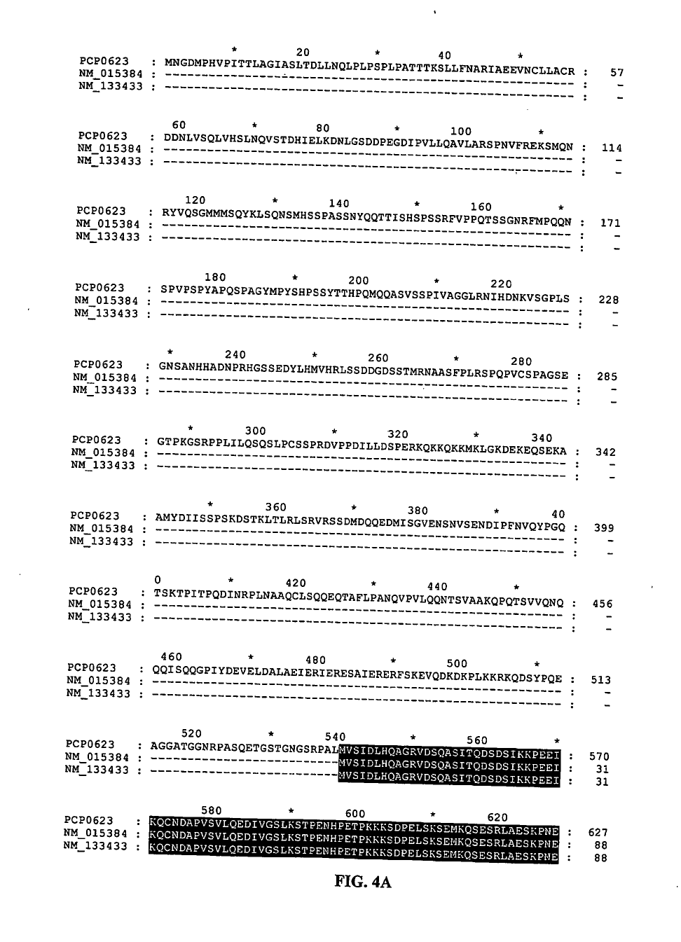

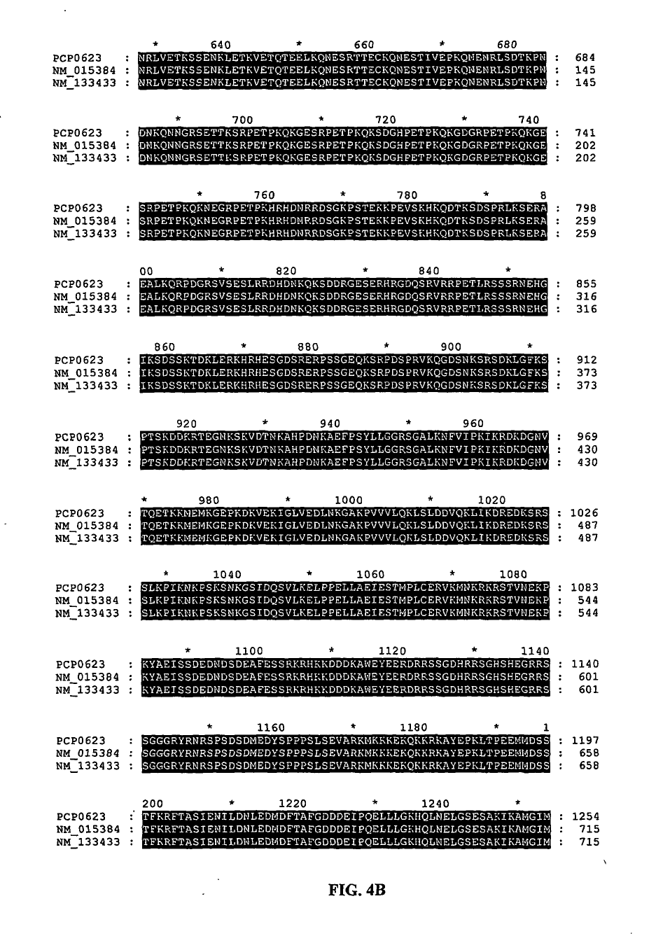

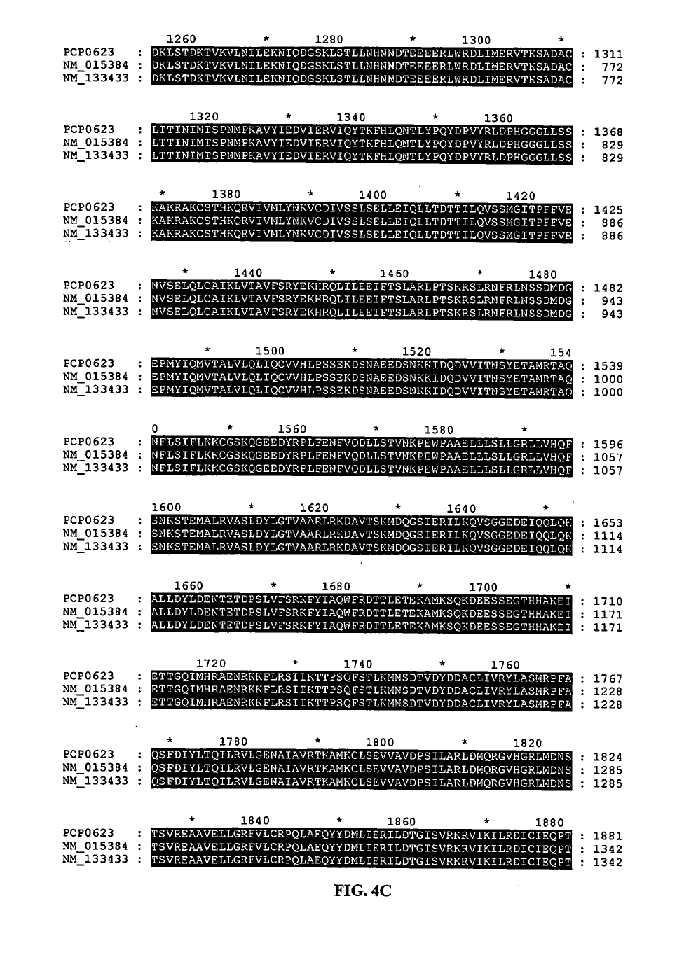

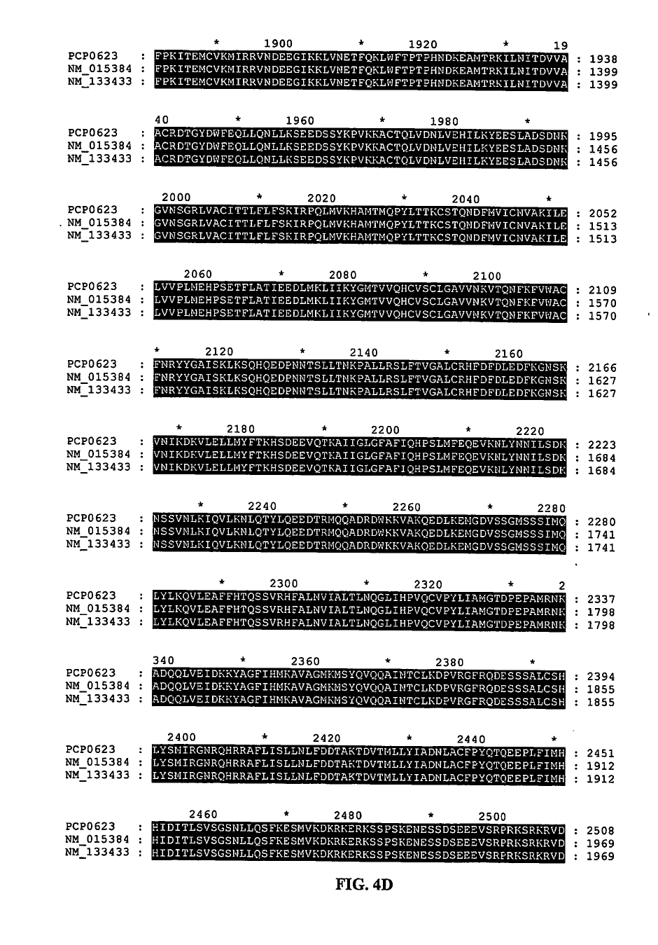

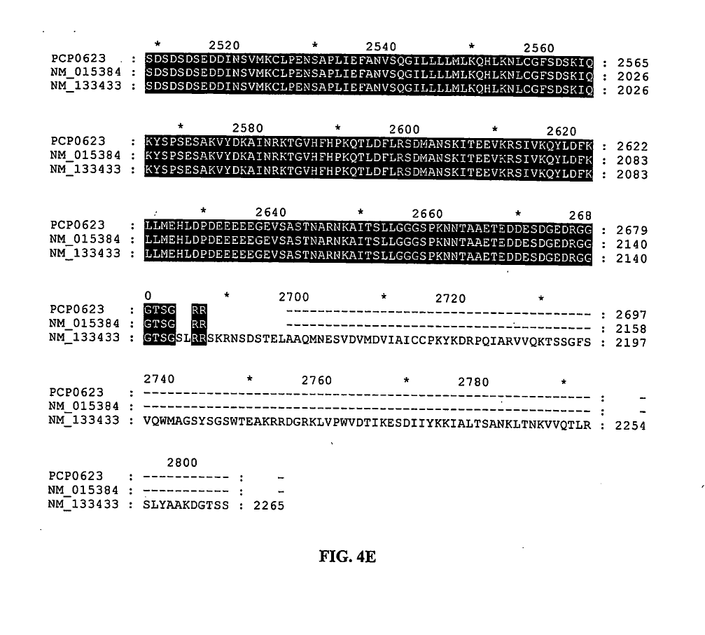

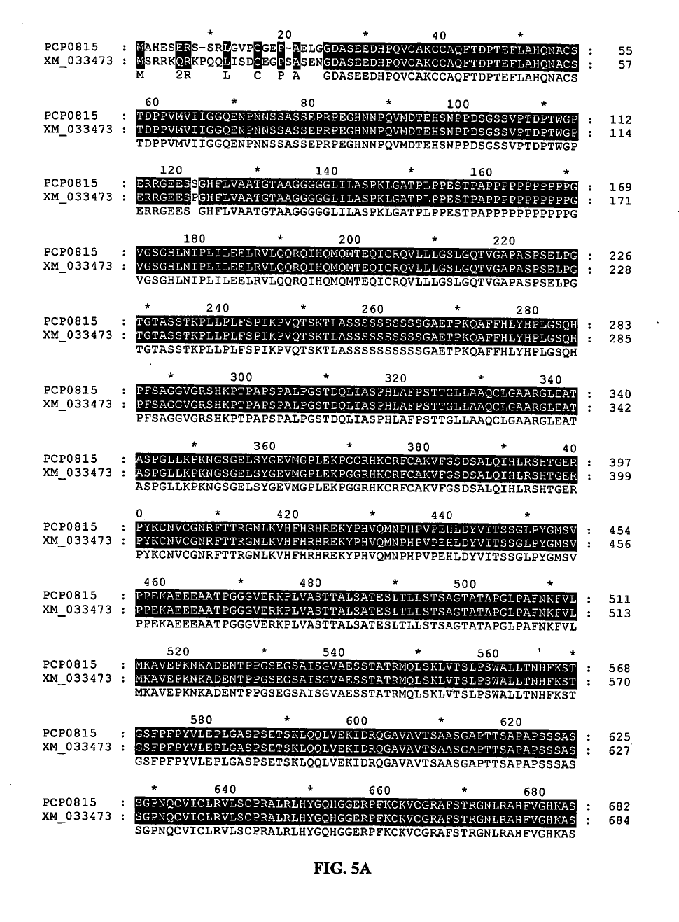

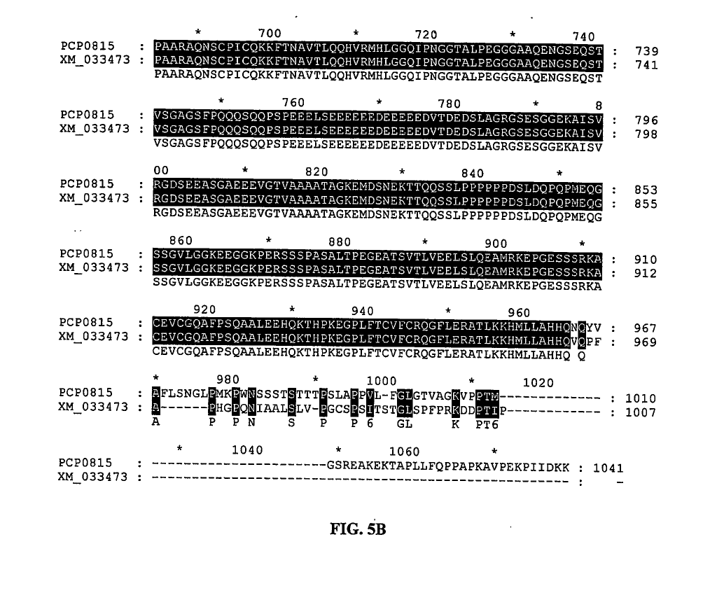

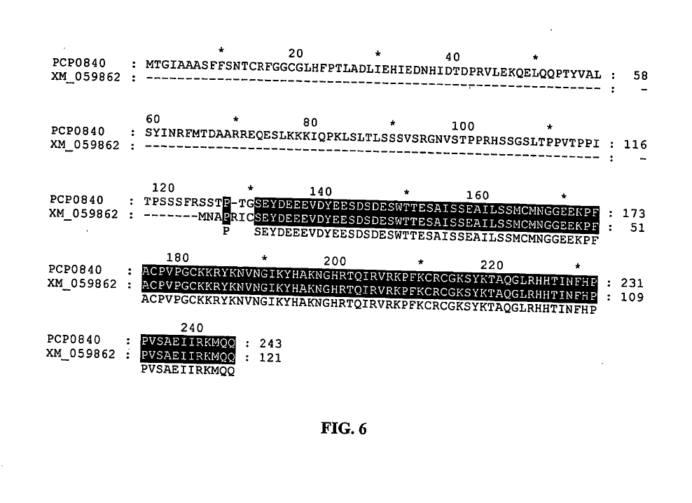

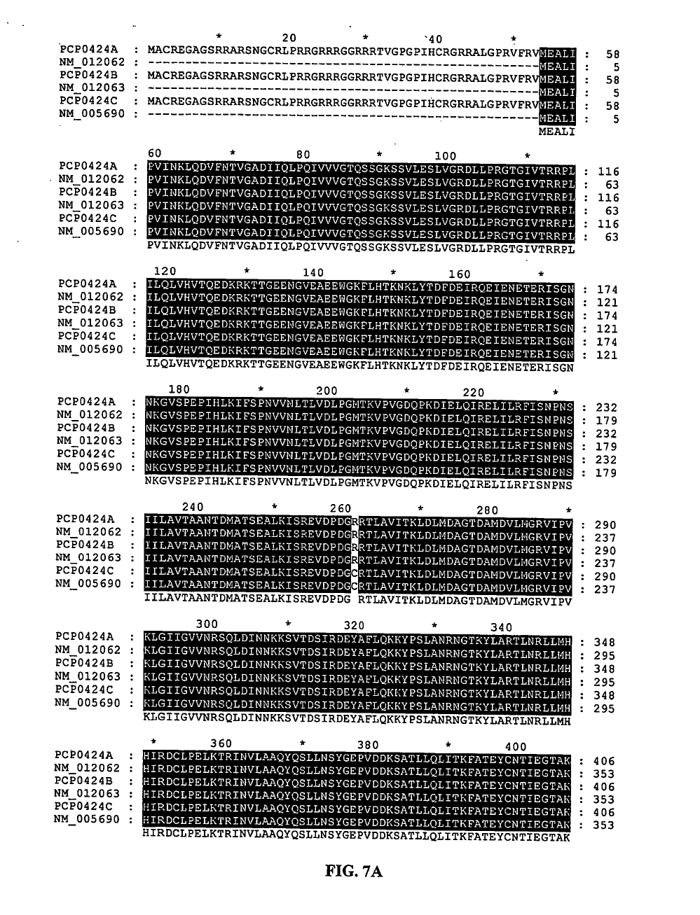

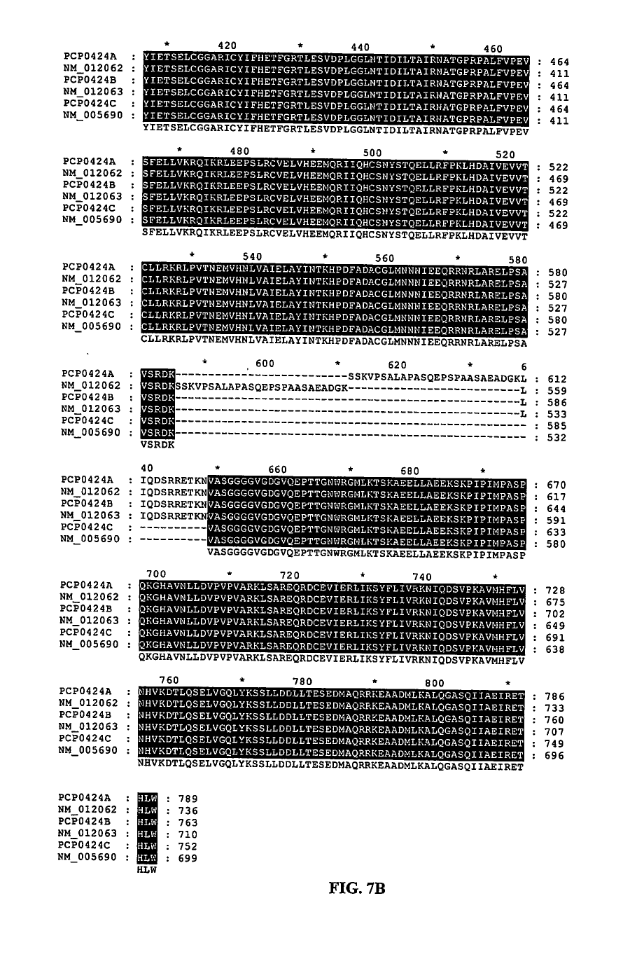

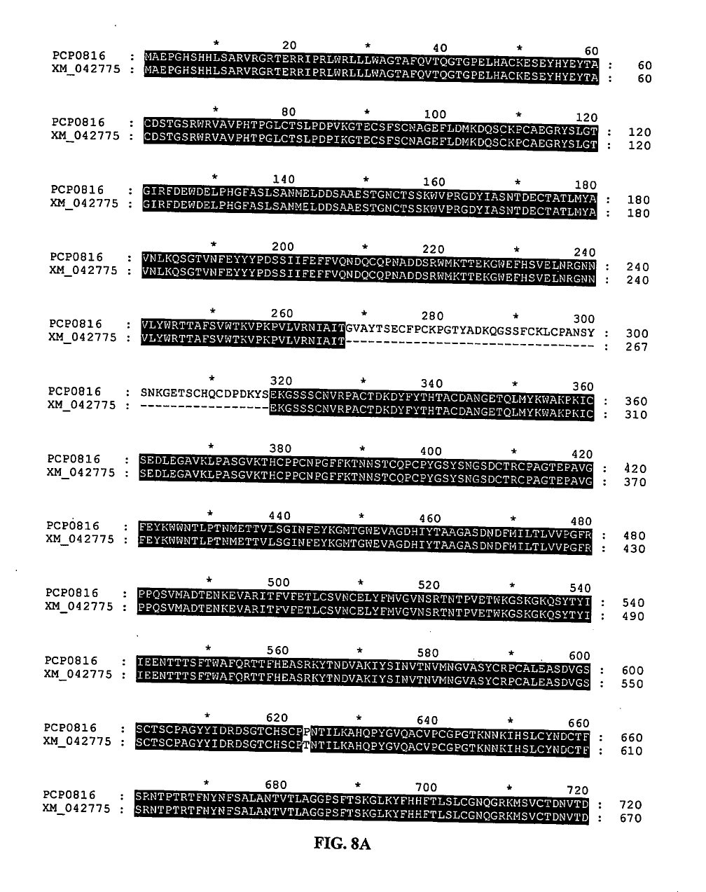

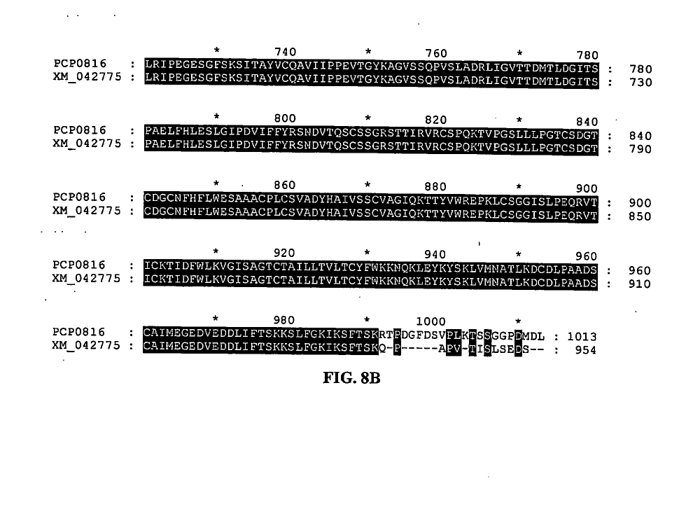

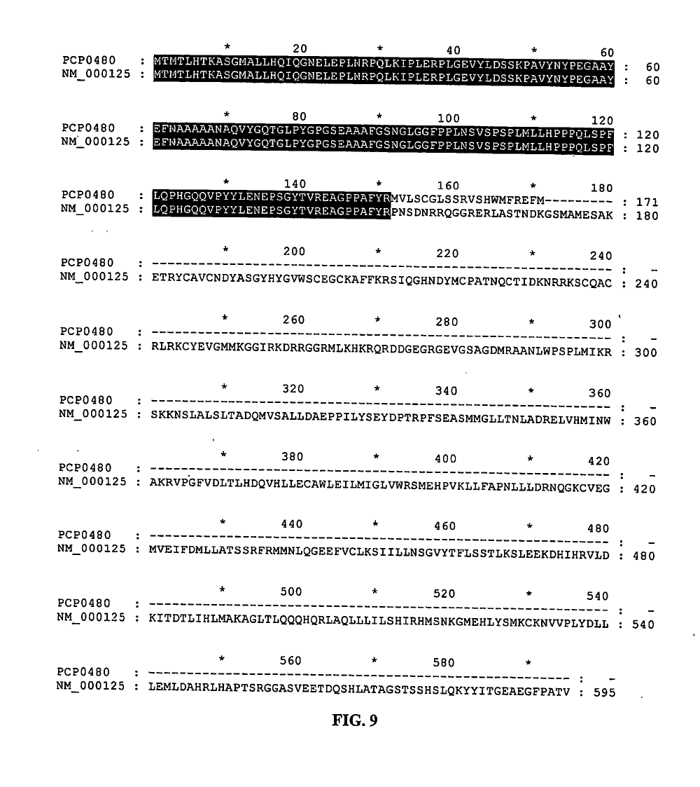

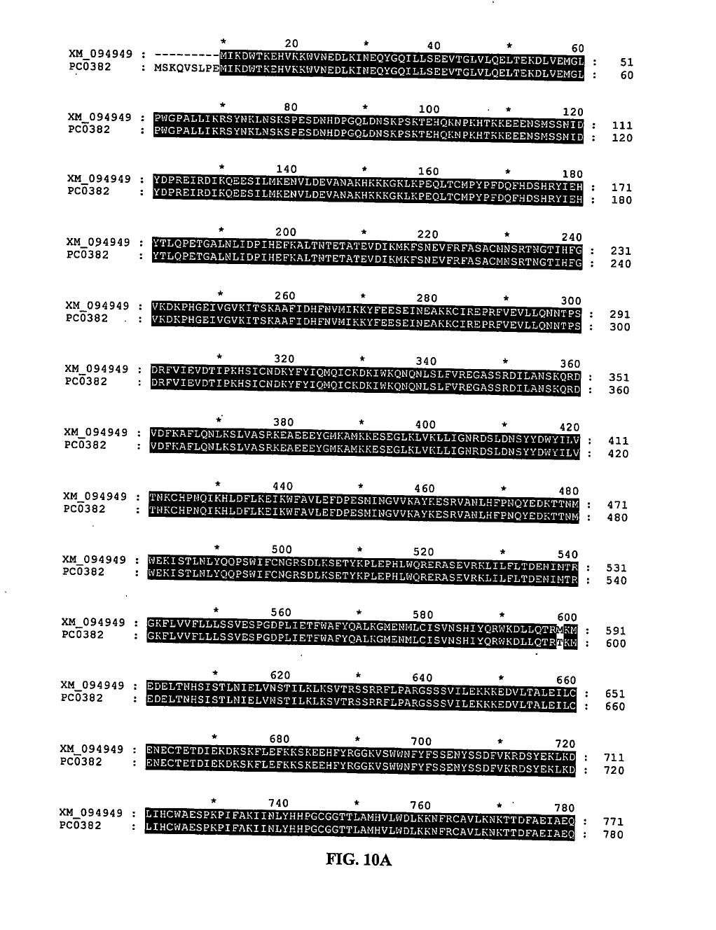

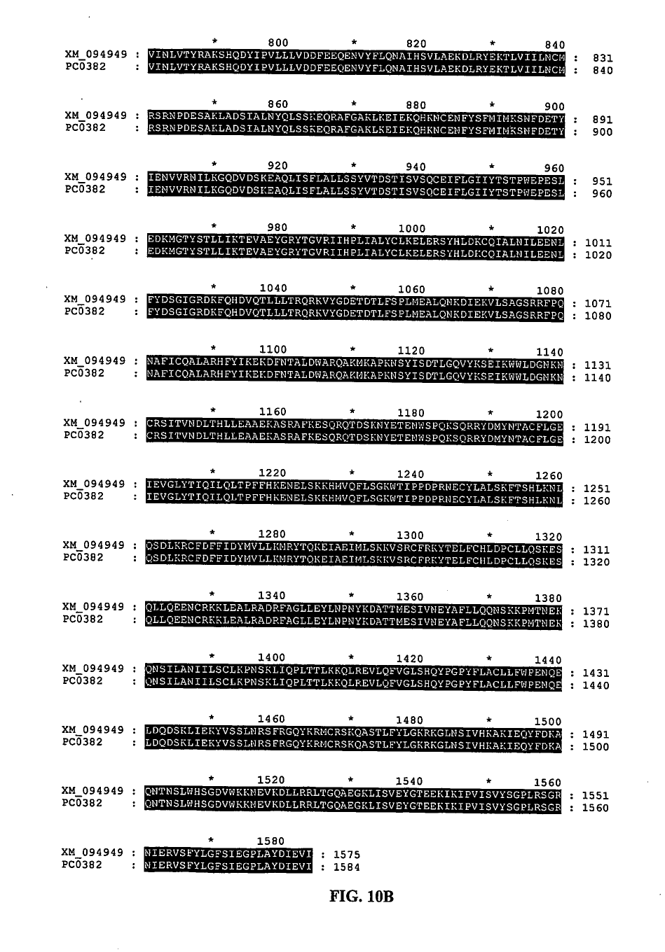

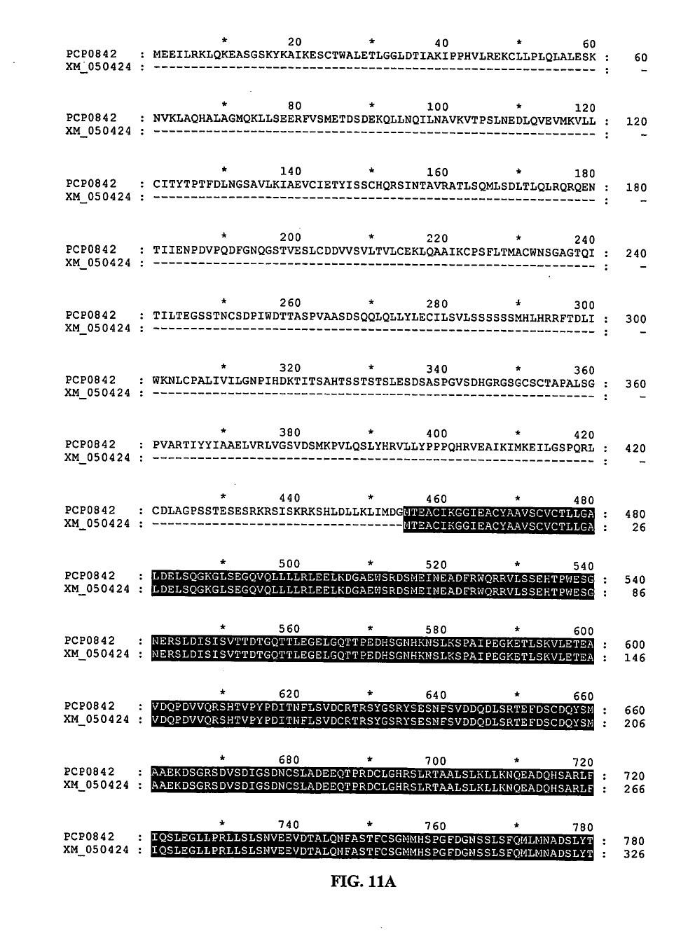

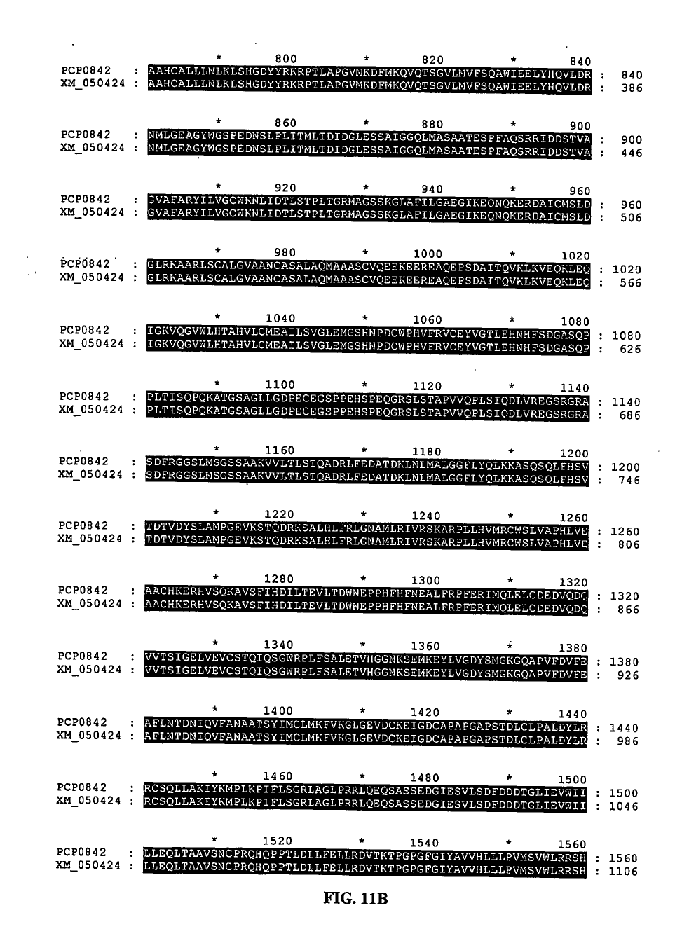

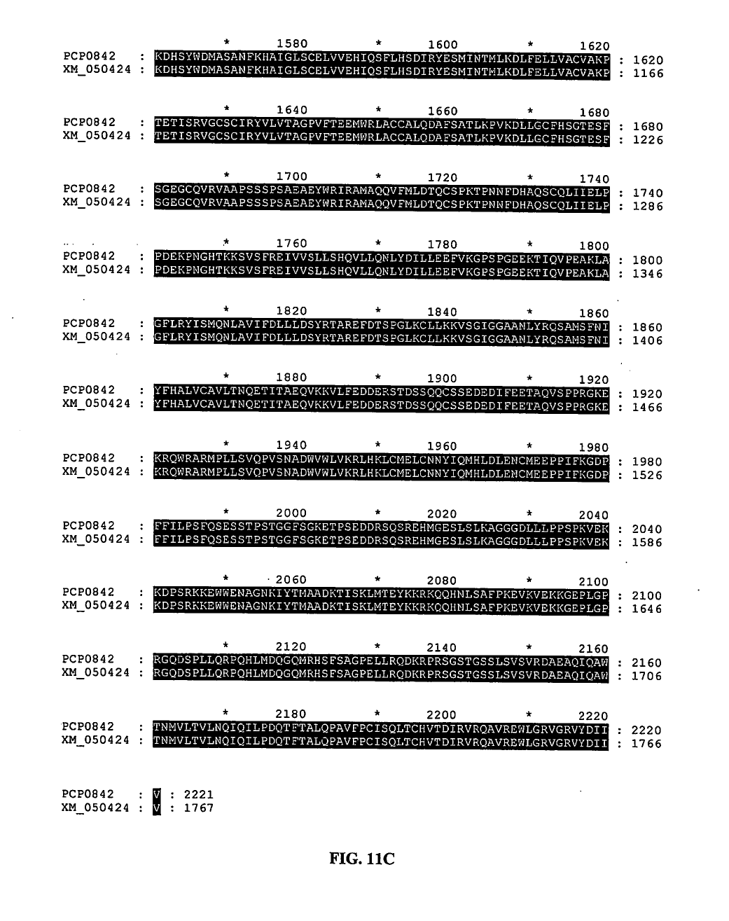

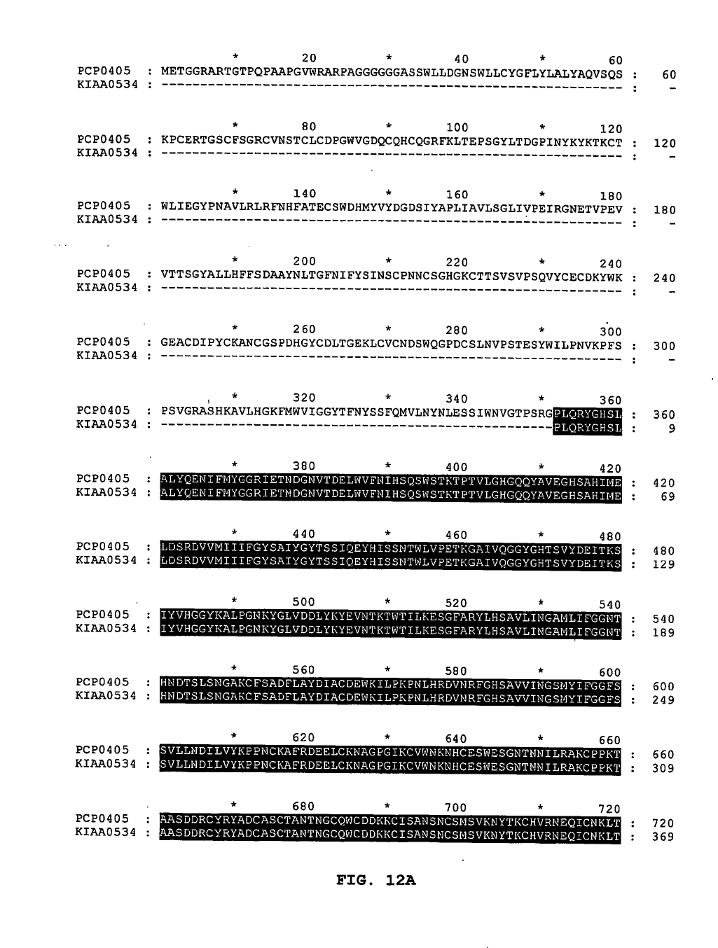

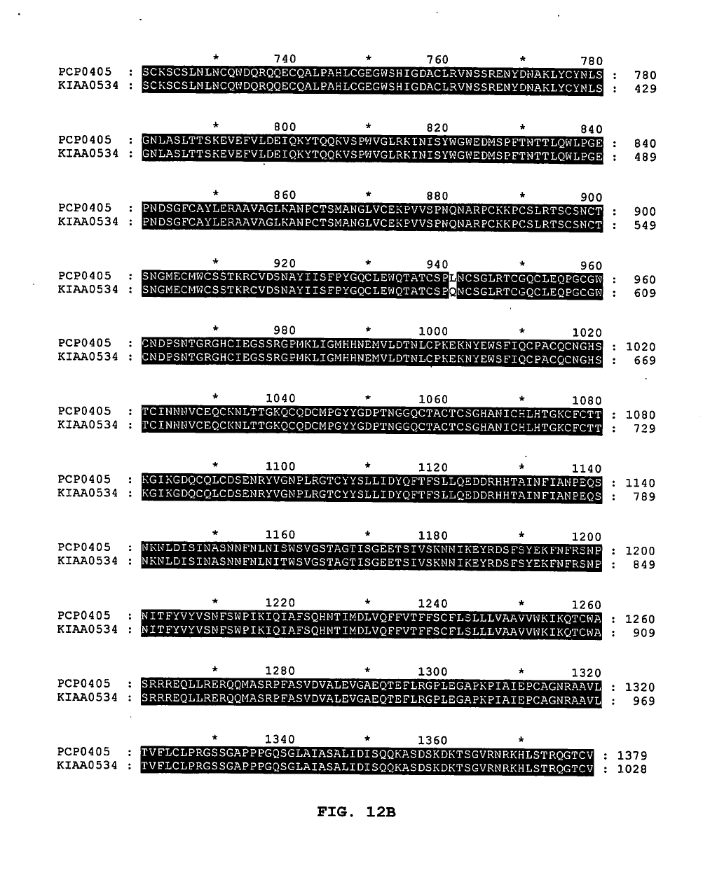

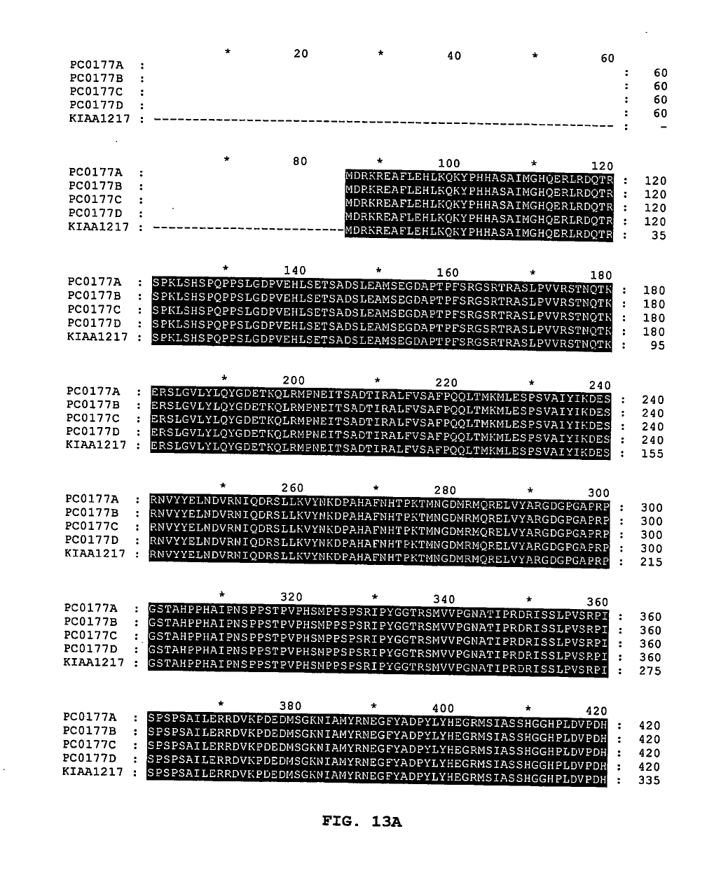

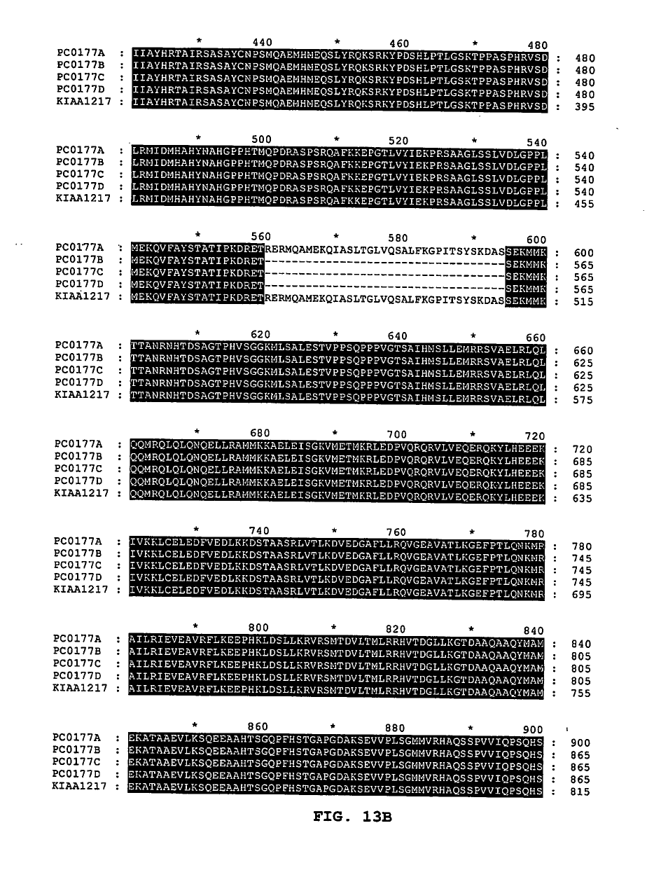

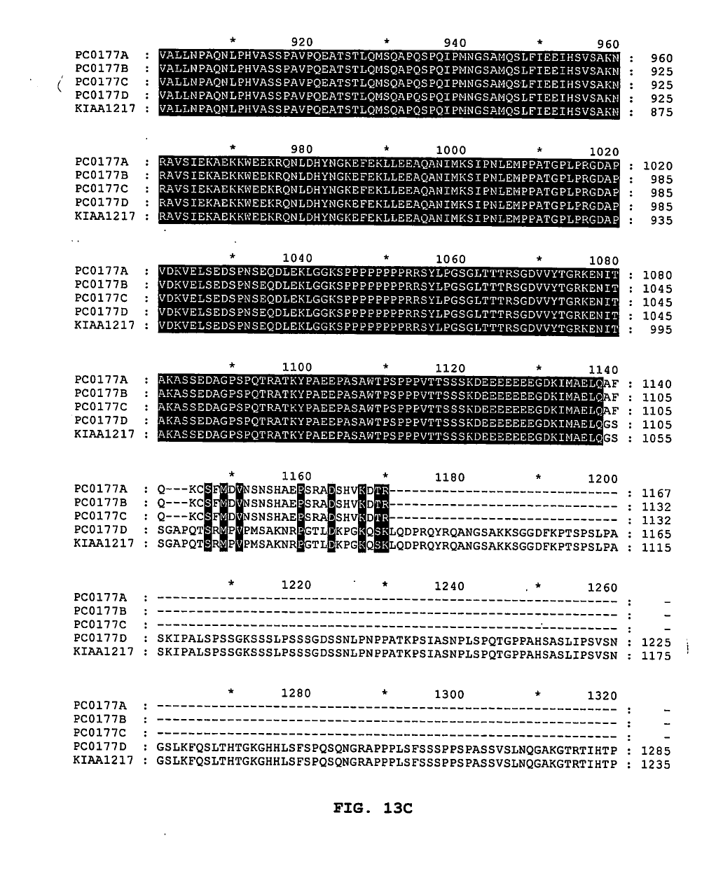

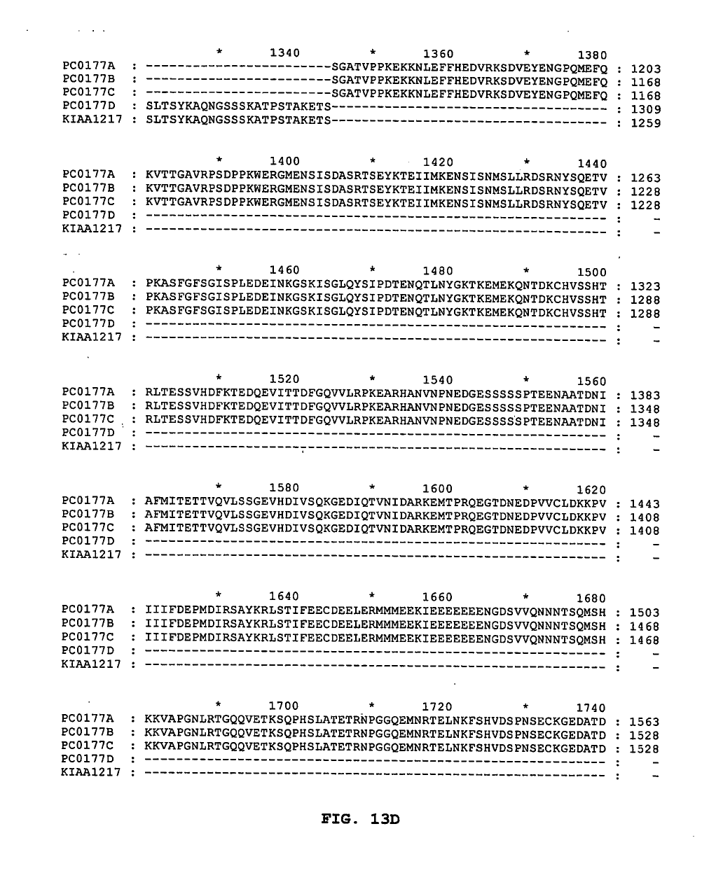

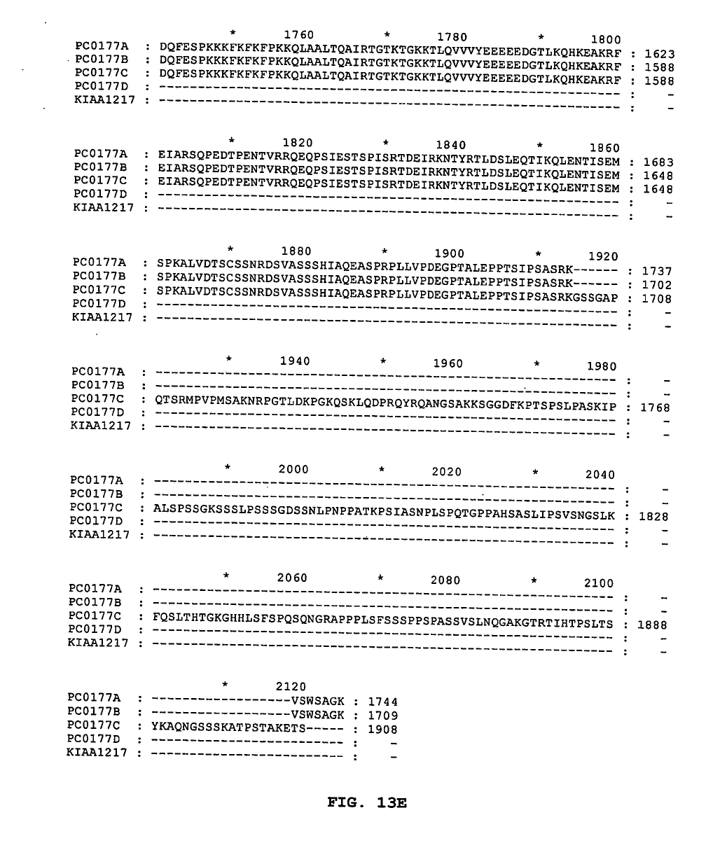

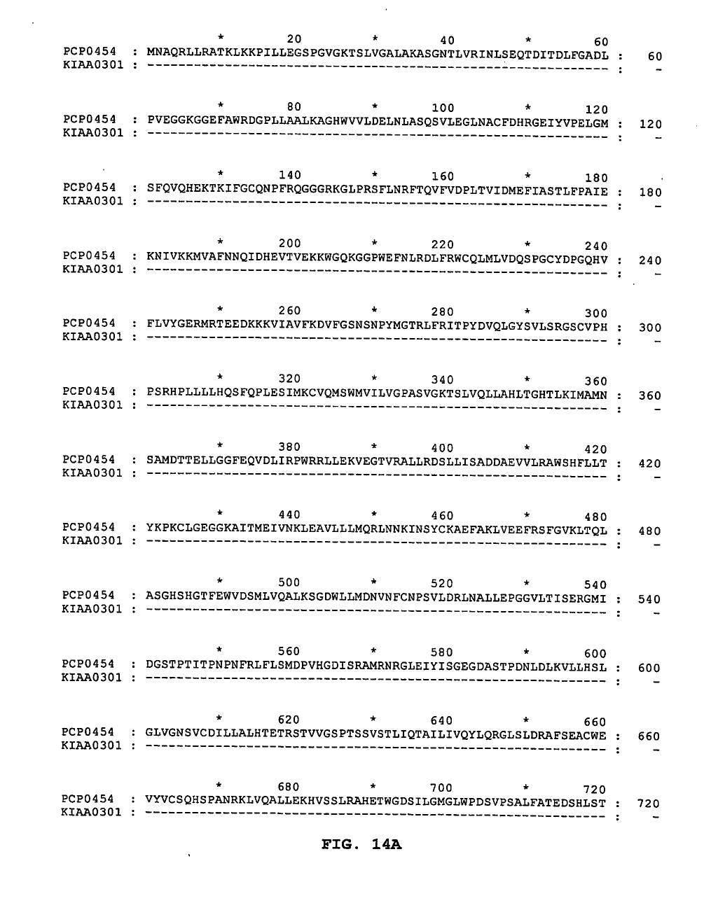

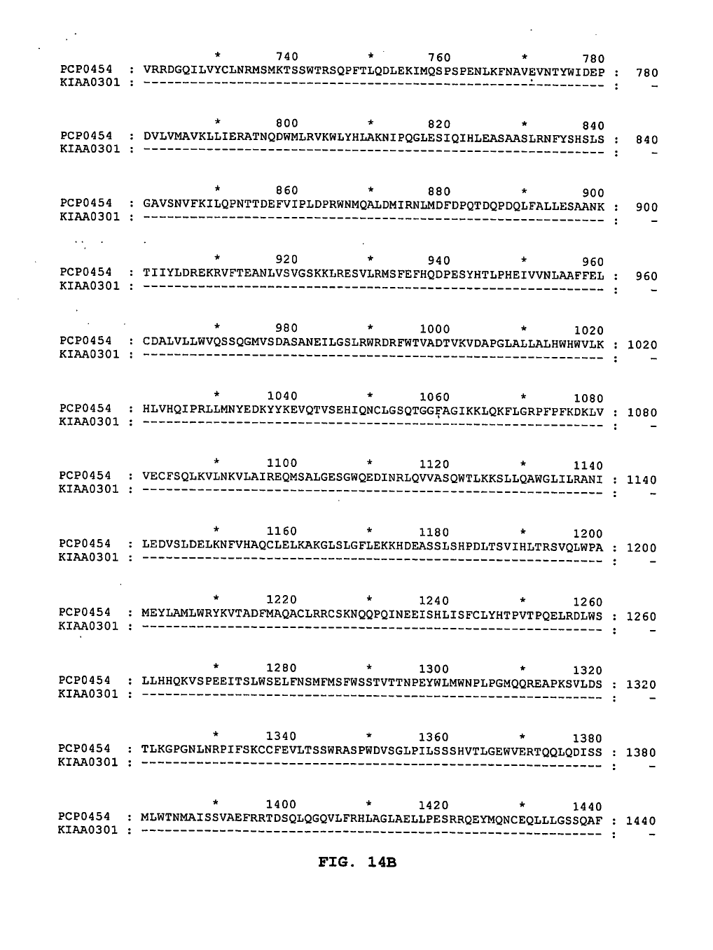

### DESCRIPTION OF THE INVENTION

The present invention relates to all facets of genes which are differentially regulated in cancer, polypeptides encoded by them, antibodies and specific binding partners thereto, and their applications to research, diagnosis, drug discovery, therapy, clinical medicine, forensic science and medicine, etc. The polynucleotides and polypeptides are useful in variety of ways, including, but not limited to, as molecular markers, as drug targets, and for detecting, diagnosing, staging, monitoring, prognosticating, preventing or treating, determining predisposition to, etc., diseases and conditions of the breast and prostate, especially cancer. The identification of specific genes, and groups of genes, expressed in pathways physiologically relevant to prostate and breast permits the definition of functional and disease pathways, and the delineation of targets in these pathways which are useful in diagnostic, therapeutic, and clinical applications. The present invention also relates to methods of using the polynucleotides and related products (proteins, antibodies, etc.) in business and computer-related methods, e.g., advertising, displaying, offering, selling, etc., such products for sale, commercial use, licensing, etc.

No single gene or protein has been identified which is responsible for the etiology of all prostate and breast cancers. For example, although prostate specific antigen (“PSA”) is widely used as a diagnostic reagent, it has limitations in its sensitivity and its ability to detect early cancers. It is estimated that approximately 20% to 30% of tumors will be missed when PSA is used alone. As a result, diagnostic and prognostic markers for cancer will involve the identification and use of many different genes and gene products to reflect its multifactorial origin With this in mind, combinations of the differentially-expressed genes of the present invention can be used as diagnostic and prognostic markers for prostate and breast cancers.

A continuing goal is to characterize the gene expression patterns of the various cancers to genetically differentiate them, providing important guidance in preventing, diagnosing, and treating cancers. Molecular pictures of cancer, such as the pattern of differentially-regulated genes identified herein, provide an important tool for molecularly dissecting and classifying cancer, identifying drug targets, providing prognosis and therapeutic information, etc. For instance, an array of polynucleotides corresponding to genes differentially regulated in prostate or breast cancer can be used to screen tissue samples for the existence of cancer, to categorize the cancer (e.g., by the particular pattern observed), to grade the cancer (e.g., by the number of up- or down-regulated genes and their amounts of expression), to identify the source of a secondary tumor, to screen for metastatic cells, etc. These arrays can be used in combination with other markers, e.g., PSA, PMSA (prostate membrane specific antigen), or any of the grading systems used in clinical medicine.

As indicated by these studies, cancer is a highly diverse disease. Although all cancers share certain characteristics, the underlying cause and disease progression can differ significantly from patient to patient. So far, over a dozen distinct genes have been identified which, when mutant, result in a cancer. In breast cancer, alone, a handful of different genes have been isolated which either cause the cancer, or produce a predisposition to it. As a consequence, disease phenotypes for a particular cancer do not look all the same. In addition to the differences in the gene(s) responsible for the cancer, heterogeneity among individuals, e.g., in age, health, sex, and genetic background, can also influence the disease and its progression. Gene penetrance, in particular, can vary widely among population members. Recent studies have shown tremendous diversity in gene expression patterns among cancer patients. For these and other reasons, one gene/polypeptide target alone can be insufficient to diagnose or treat a cancer. Even a gene which is highly differentially-expressed and penetrant in cancer patients may not be so highly expressed in all patients and at all stages of the cancer. By selecting a set of genes and/or the polypeptides they encode, cancer diagnostics and therapeutics can be designed which effectively diagnose and treat a population of diseased individuals, rather than only a small handful when single genes are targeted.

In accordance with the present invention, genes have been identified which are differentially expressed in prostate cancer. See, e.g., Tables 1-5 and below. These genes can be further divided into groups based on additional characteristics of their expression and the tissues in which they are expressed. For instance, genes can be further subdivided based on the stage and/or grade of the cancer in which they are expressed. Genes can also be grouped based on their penetrance in a cancer, e.g., expressed in all cancer examined, expressed in a certain percentage of cancers examined, etc. Additionally, genes can be categorized by their function and/or the polypeptides they encode. This includes, but is not limited to, cellular localization, functional activity (e.g., kinase, cytoskeletal element, or transcriptional factor), functional pathway (e.g., protein manufacture, cell signaling, cell movement, cell adhesion, responsivity to cAMP, energy production, etc.), etc. These groupings do not restrict or limit the use such genes in therapeutic, diagnostic, prognostic, etc., applications. For instance, a gene which is expressed in only some cancers (e.g., incompletely penetrant) may be useful in therapeutic applications to treat a subset of cancers. Similarly, a co-penetrant gene, or a gene which is expressed in prostate cancer and other normal tissues, maybe useful as a therapeutic or diagnostic, even if its expression pattern is not highly prostate specific. Thus, the uses of the genes or their products are not limited by their patterns of expression.

In developing reagents for the diagnosis and treatment of a disease, it may be useful to know the cellular localization of a differentially expressed polypeptide to determine how to use it as a target. Proteins which are secreted or on the cell-surface are more readily accessible than intracellular proteins, and can be, e.g., blocked or inhibited to restore levels to normal.

In recent years, there have been numerous reports on specific targeting of tumor cells with monoclonal antibody-drug conjugates using cell-surface proteins. See, e.g., Chari., Adv. Drug Deliv. Res., 31: 89-104 (1998); Pietersz and Krauer, J. Drug Targeting, 2: 183-215 (1994); Sela et al., in Immunoconjugates, 189-216 (C. Vogel, ed. 1987); Ghose et al., in Targeted Drugs, 1-22 (E. Goldberg, ed. 1983); Diener et al., in Antibody mediated delivery systems, 1-23 (J. Rodwell, ed. 1988); Pietersz et al., in Antibody mediated delivery systems, 25-53 (J. Rodwell, ed. 1988); Bumol et al., in Antibody mediated delivery system, 55-79 (J. Rodwell, ed. 1988). Cytotoxic drugs such as methotrexate, daunorubicin, doxorubicin, vincristine, vinblastine, melphalan, mitomycin C, and chlorambucil have been conjugated to a variety of monoclonal antibodies. Therapeutic agents can be directly conjugated to the antibody, or through cleavable linkers which facilitate the release of the agent in active form only when it is inside the cell. See, e.g., U.S. Pat. No. 6,333,410.

By the phrase “differential expression,” it is meant that the levels of expression of a gene, as measured by its transcription or translation product, are different depending upon the specific cell-type or tissue (e.g., in an averaging assay that looks at a population of cells). There are no absolute amounts by which the gene expression levels must vary, as long as the differences are measurable.

The phrase “up-regulated” indicates that an mRNA transcript or other nucleic acid corresponding to a polynucleotide of the present invention is expressed in larger amounts in a cancer as compared to the same transcript expressed in normal cells from which the cancer was derived. The phrase “down-regulated” indicates that an mRNA transcript or other nucleic acid corresponding to a polynucleotide of the present invention is expressed in lower amounts in a cancer as compared to the same transcript expressed in normal cells from which the cancer was derived. In general, differential-regulation can be assessed by any suitable method, including any of the nucleic acid detection and hybridization methods mentioned below, as well as polypeptide-based methods. Up-regulation also includes going from substantially no expression in a normal tissue, from detectable expression in a normal tissue, from significant expression in a normal tissue, to higher levels in the cancer. Down-regulation also includes going from substantially no expression in a normal tissue, from detectable expression in a normal tissue, from significant expression in a normal tissue, to higher levels in the cancer.

Differential regulation can be determined by any suitable method, e.g., by comparing its abundance per gram of RNA (e.g., total RNA, polyadenylated mRNA, etc.) extracted from a prostate tissue in comparison to the corresponding normal tissue. The normal tissue can be from the same or different individual or source. For convenience, it can be supplied as a separate component or in a kit in combination with probes and other reagents for detecting genes. The quantity by which a nucleic acid is differentially-regulated can be any value, e.g., about 10% more or less of normal expression, about 50% more or less of normal expression, 2-fold more or less, 5-fold more or less, 10-fold more or less, etc.

The amount of transcript can also be compared to a different gene in the same sample, especially a gene whose abundance is known and substantially no different in its expression between normal and cancer cells (e.g., a “control” gene). If represented as a ratio, with the quantity of differentially-regulated gene transcript in the numerator and the control gene transcript in the denominator, the ratio would be larger, e.g., in prostate cancer than in a sample from normal prostate tissue.

Differential-regulation can arise through a number of different mechanisms. The present invention is not bound by any specific way through which it occurs. Differential-regulation of a polynucleotide can occur, e.g., by modulating (1) transcriptional rate of the gene (e.g., increasing its rate, inducing or stimulating its transcription from a basal, low-level rate, etc.), (2) the post-transcriptional processing of RNA transcripts, (3) the trasport of RNA from the nucleus into the cytoplasm, (4) RNA nuclear and cytoplasmic turnover, and polypeptide turnover (e.g., by virtue of having higher stability or resistance to degradation), and combinations thereof See, e.g., Tollervey and Caceras, Cell, 103:703-709, 2000.

A differentially-regulated polynucleotide is useful in a variety of different applications as described in greater details below. Because it is more abundant in cancer, it and its expression products can be used in a diagnostic test to assay for the presence of cancer, e.g., in tissue sections, in a biopsy sample, in total RNA, in lymph, in blood, etc. Differentially-regulated polynucleotides and polypeptides can be used individually, or in groups, to assess the cancer, e.g., to determine the specific type of cancer, its stage of development, the nature of the genetic defect, etc., or to assess the efficacy of a treatment modality. How to use polynucleotides in diagnostic and prognostic assays is discussed below. In addition, the polynucleotides and the polypeptides they encode, can serve as a target for therapy or drug discovery. A polypeptide, coded for by a differentially-regulated polynucleotide, which is displayed on the cefl-surface, can be a target for immunotherapy to destroy, inhibit, etc., the diseased tissue. Differentially-regulated transcripts can-also be used in drug discovery schemes to identify pharmacological agents which modulate, suppress, inhibit, activate, increase, etc., their differential-regulation, thereby preventing the phenotype associated with their expression. Thus, a differentially-regulated polynucleotide and its expression products of the present invention have significant applications in diagnostic, therapeutic, prognostic, drug development, and related areas.

The expression patterns of the selectively expressed genes disclosed herein can be described as a “fingerprint” in that they are a distinctive pattern displayed by a tissue. Just as with a fingerprint, an expression pattern can be used as a unique identifier to characterize the status of a tissue sample. The list of expressed sequences disclosed herein provides an example of such a tissue expression profile. It can be used as a point of reference to compare and characterize samples. Tissue fingerprints can be used in many ways, e.g., to classify a tissue as prostate cancer, to determine the origin of a metastatic cells, to assess the physiological status of a tissue, to determine the effect of a particular treatment regime on a tissue, and to evaluate the toxicity of a compound on a tissue of interest, to determine the presence of a cancer in a biopsy sample, to assess the efficacy of a cancer therapy in a human patient or a non-human animal model, to detect circulating cancer cells in blood or a lymph node biopsy, etc. While the expression profile of the complete gene set represented in the present invention may be most informative, a fingerprint containing expression information from less than the full collection can be useful, as well. In the same way that an incomplete fingerprint may contain enough of the pattern of whorls, arches, loops, and ridges, to identify the individual, a cell expression fingerprint containing less than the full complement may be adequate to provide useful and unique identifying and other information about the sample. Moreover, cancer is a multifactorial disease, involving genetic aberrations in more than gene locus. This multifaceted nature may be reflected in different cell expression profiles associated with prostate cancers arising in different individuals, in different locations in the same individual, or even within the same cancer locus. As a result, a complete match with a particular cell expression profile, as shown herein, is not necessary to classify a cancer as being of the same type or stage. Similarity to one cell expression profile, e.g., as compared to another, can be adequate to classify cancer types, grades, and stages.

For example, the tissue-selective genes disclosed herein represent the configuration of genes expressed by a cancer tissue. To determine the effect of a toxin on a tissue, a sample of tissue is obtained prior to toxin exposure (“control”) and then at one or more time points after toxin exposure (“experimental”). An array of tissue-selective probes can be used to assess the expression patterns for both the control and experimental samples. Methods of making and using arrays are described below.

Urb-ctf (BCU1041FB)

Urb-ctf (“Up-Regulated Breast Cancer Transcription Factor” or BCU1041FB or FB2847A11) codes for a transcription regulatory factor having 614 amino acids which is up-regulated in breast cancer. The nucleotide and amino acid sequences of Urb-ctf are shown in SEQ ID NOS 95 and 96. It contains a bZIP domain at about amino acid positions 228-275, conferring DNA-binding activity. It also has a leucine zipper providing a dimerization activity. There are a number of UniGene clusters that map close to the gene, including, e.g., Hs.350229, Hs.272458, Hs.350229, Hs.255286, Hs.184779, and Hs.276916. Predictions using GenomeScan (e.g., Yeh et al., Genome Res. 11: 803-816, 2001) revealed at least two different predicted genes, Hs17_{—}11001_{—}27_{—}4_{—}1 and Hs17_{—}11001_{—}27_{—}5_{—}2, instead of the single gene, Urb-ctf, described herein. A partial human cDNA (AL049450; XM_{—}058887; SEQ ID NO 97) for Urb-ctf was previously identified, but this coded for only 198 amino acids and contained only a part of the bZIP domain, as well as missing significant portions of the N- and C-termini. A mouse homolog, AK_{—}014463 (SEQ ID NO 98), has been cloned.

All or part of Urb-ctf is located in genomic DNA represented by GenBank ID: AC068669, BAC-ID: RP11-749116, and Contig ID: NT_{—}010844. The present invention relates to any isolated introns and exons that are present in the gene. Intron and exon boundaries can be routinely determined, e.g., using the polypeptide and genomic sequences disclosed herein. Using UniSTS probes, Urb-ctf can be chromosomally mapped at its 5′ end with UniSTS: 155813 to 40.144 Mb, and its 3′ end with UniSTS: 619 to 40.084 Mb. Strikingly, the Urb-ctf overlaps with the thyroid hormone receptor alpha 2 gene (CAB57886).

As indicated by the presence of a bZIP domain, Urb-ctf has transcriptional regulatory activity, DNA-binding activity, and dimerization activity. These activities can be determined routinely. For example, DNA-binding activity can be determined using gel-shift assays, e.g., as carried out in, e.g., U.S. Pat. No. 6,333,407 and 5,789,538. Transcriptional activity can be determined using conventional transcriptional assays, including in vivo and in vitro assays, such as those described in F. M. Ausubel et al., Eds., CURRENT PROTOCOLS IN MOLECULAR BIOLOGY (John Wiley & Sons, New York, 1994); de Wet et al., Mol. Cell Biol. 7:725 (1987); U.S. Pat 6,306,649; U.S. Pat. No. 6,214,588; Liao, S. M. et al., Genes. Dev. 5:2431-2440 (1991); Nonet, M., et al., Cell 50:909-915 (1987). The phrase “transcriptional regulatory activity” indicates that the polypeptide modulates transcription in analogy to the activity of other bZIP proteins, e.g., by binding to DNA and interacting with other proteins of the transcription apparatus. For example, both c-Jun and c-Fos are bZIP proteins that form a dimer known as the transcriptional activator AP-1, a transcriptional activator. See, e.g., Genes VII, Lewin, Pages 649-665, 2000. Dimerization activity, i.e., the ability to form hetero- or homodimers with other proteins (in analogy to the c-fos and c-jun system), can be measured routinely, e.g., using the yeast two-hybrid system.

Nucleic acids of the present invention map to chromosomal band 17q21.1. There are a number of different disorders which have been mapped to, or in close proximity to, this chromosome location. These include, e.g., Dementia, frontotemporal, with parkinsonism; Neuroblastoma; Osteoporosis, idiopathic; Ehlers-Danlos syndrome, types I and VIIA; Osteogenesis imperfecta; Glanzmann thrombasthenia, type B; Renal cell carcinoma, papillary; Thrombocytopenia, neonatal alloimmune; Trichodontoosseous syndrome; Hypertension; Epidermolytic hyperkeratosis; Hemolytic anemia due to band 3 defect; Spherocytosis, hereditary; Gliosis, familial progressive subcortical; Renal tubular acidosis, distal; Patella aplasia or hypoplasia; and Pseudohypoaldosteronism type II. Nucleic acids of the present invention can be used as linkage markers, diagnostic targets, therapeutic targets, for any of the mentioned disorders, as well as any disorders or genes mapping in proximity to it.

In addition to its expression in breast cancer, Urb-ctf can be detected in most tissues examined, but either none, or at very low levels, in normal breast tissue. Multiple forms of it can be detected in the brain, muscle, testes, and thymus. As these results indicate, Urb-ctf has a normal functional role in most tissues, and can consequently be involved with diseases associated with them, as well. For instance, Urb-ctf can be involved in renal cell carcinoma and familial gliosis disease. As discussed earlier, no single gene is responsible for all breast cancers. Thus, the fact that Urb-ctf is up-regulated in the breast cancers examined herein does necessarily mean that it will be up-regulated in all human breast cancers.

BCU399

Human BCU399 codes for a polypeptide of 487 amino acids, which is upregulated in breast cancer, in both early stage ductile carcinoma and in late stage invasive carcinoma The nucleotide and amino acid sequences of human BCU399 are shown in SEQ ID NOS 99 and 100. It contains seven transmembrane domains at about amino acids 106-128, 135-157, 172-194, 231-253, 268-285, 367-389 and 458480 of SEQ ID NO 100, a signature of the G protein-coupled receptor family. It contains a nucleotide-binding site motif (P-loop) at about amino acids 53-60, indicating that it is a purinergic receptor liganded by nucleotides, including ATP, ADP, GTP, GDP, UTP, and/or UDP. It contains a G-protein binding motif at about amino acids 63-75. It contains N-glycosylation motifs at about amino acids 34-37, 135-138, 203-206 and 397400. It contains phosphorylation motifs, important for regulatory functions, including PKC phosphorylation motifs at about amino acids 36-38, 227-229, 262-264, 313-315, and 445-447 of SEQ ID NO 100; CK2 phosphorylation motifs at about amino acids 44-47, 60-63, 89-92,91-94, and 356-359; and a tyrosine kinase phosphorylation motif at about amino acids 59-66. The human BCU399 contains 12 exons. The present invention relates to any isolated introns and exons that are present in the gene. Intron and exon boundaries can be routinely determined, e.g., using the sequences disclosed herein.

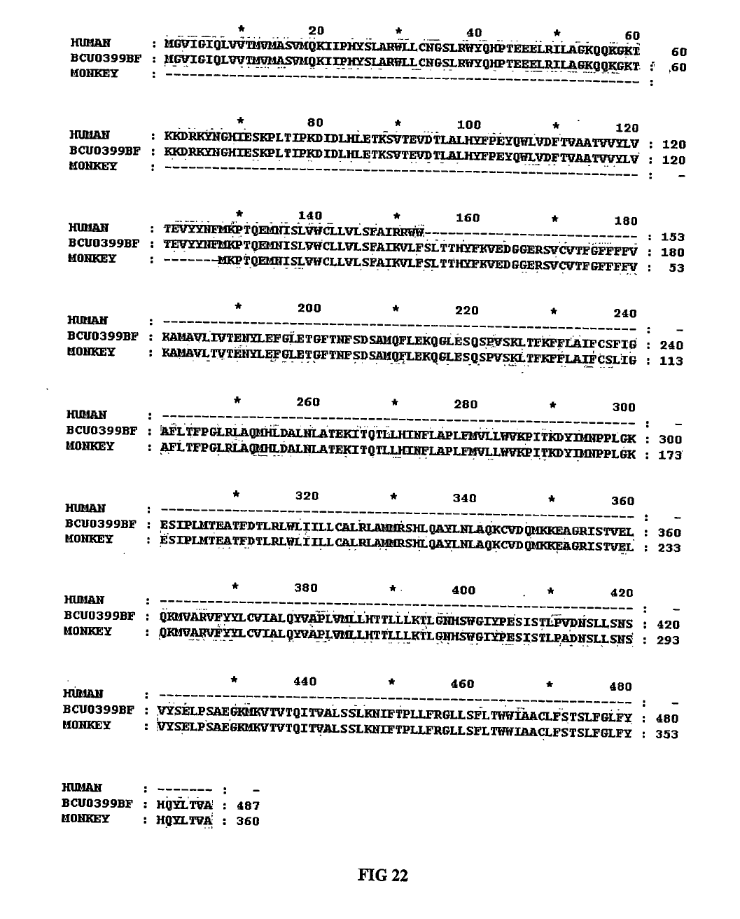

A partial sequence for human BCU399 was previously identified (Accession Number XM_{—}059670), but this sequence (SEQ ID 101) was incomplete, coding for only 153 amino acids (See FIG. 22, “Human”). Its homolog was identified in monkey (Accession Number AB071059), but this sequence was also incomplete, coding for only 360 amino acids (See FIG. 22, “Monkey”). Monkey BCU399 (SEQ ID NO 102) lacks the first 127 amino acids of human BCU399 (SEQ ID NO 100) but shares about 99% amino acid sequence identity to human BCU399 along its entire length of 360 amino acids, with three amino acids different from human BCU399 at about positions 187,238, and 412 (See FIG. 22). Related genes have been identified in human, mouse, and Drosophila. Bcu0399 shares 48% identity with the related full-length human sequence XM_{—}009330; 46% identity with its mouse homolog BC021367; and 41% identity with its fly homolog AE003546. The functions of these homologs are unknown, and all three lack the nucleotide-binding site of BCU399, indicating that they are functionally different from BCU399.

Because of the upregulation of BCU399 in breast cancer tissue, its polynucleotides, polypeptides, and peptides can be used as diagnostic, therapeutic, and research tools in breast cancer. Upregulation can be routinely assessed by, e.g., RT-PCR. Antibodies and other BCU399 ligands can be used to selectively target agents to breast tissue for purposes including, but not limited to, imaging, diagnostic, therapeutics, etc. In addition to its association with breast cancer, BCU399 is also expressed in lymphocytes and in adrenal, brain, kidney, lung, lymph node, breast, muscle, ovary, prostate, stomach, testis, thymus and thyroid tissue.

Imaging of tissues can be facilitated using agents such as BCU399 ligands that can be used to target contrast agents to a specific site in the body. Various imaging techniques have been used in this context, including, e.g., X-ray, CT, CAT, MRI, ultrasound, PET, SPECT, and scintographic. A reporter agent can be conjugated or associated routinely with a BCU399 ligand. Ultrasound contrast agents combined with ligands such as antibodies are described in, e.g., U.S. Pat. Nos 6,264,917; 6,254,852; 6,245,318; and 6,139,819. MRI contrast agents, such as metal chelators, radionucleotides, paramagnetic ions, etc., combined with selective targeting agents are also described in the literature, e.g., in U.S. Pat. Nos. 6,280,706 and 6,221,334. The methods described therein can be used generally to associate BCU399 and ligands thereof with an agent for any desired purpose.

An active agent can be associated in any manner with a BCU399 ligand that is effective to achieve its delivery to the target. The association of the active agent and the ligand (“coupling”) can be direct, e.g., through chemical bonds between the binding ligand and the agent or via a linking agent, or the association can be less direct, e.g., where the active agent is in a liposome, or other carrier, and the ligand is associated with the liposome surface. In such case, the ligand can be oriented in such a way that it is able to bind to BCU399 on the surfaces of breast tissue cells.

BCU399 maps to the chromosomal region 5q14.3. Consistent with its neuronal expression, a susceptibility to febrile seizures (Febrile Convulsions, Familial, 4) was mapped to this same chromosomal locus. Nakayama et al., Human Molecular Genetics, 9:87-91, 2000. In addition, several other diseases mapped to this location, including, e.g., Wagner syndrome and Usher syndrome, both disorders involved in eye disease. Black et al., Ophthalmology, 106:2074-2081, 1999. Pieke-Dahl et al., Journal of Medical Genetics, 37:256-262, 2000. Nucleic acids of the present invention can be used, e.g., as linkage markers, diagnostic targets, and therapeutic targets for any of the mentioned disorders, as well as any disorders or genes mapping in proximity of BCU399.

Its nucleotide binding properties make BCU399 polypeptides useful for assaying nucleotides such as ATP, GTP, etc. Various assay methods can be used, including filtration assays, column chromatography, etc. where labeled BCU399 polypeptides and/or nucleotides are used. BCU399 polypeptides or portions thereof including, e.g., the nucleotide binding motif and other motifs important in nucleotide binding can be used as a capture moiety. Various detection methods can be used. For example, nucleotide binding and relative concentration can be measured spectroscopically (e.g., EPR spectroscopy). BCU399 polypeptides or portions thereof can also be incorporated into column chromatography resins. After binding to the column resin, nucleotides can be chemically released and measured by commercially available bioluminescence assays (e.g., BioWhittaker ViaLight HS kit). Competitive binding assays can also be utiled, where concentration in an unknown sample is determined by its ability to compete with labeled nucleotide.

Useful human BCU399 polypeptides and corresponding nucleic acids include polypeptides comprising amino acids 1-127, 150487, 170-200, 230-250, 400420 and fragments thereof (See SEQ ID NO 100 and FIG. 22). Useful human BCU399 polypeptides and corresponding nucleic acids also include the nucleotide binding motif at about amino acids 53-60; extra-membrane loop sequences at about amino acids 1-105, 129-134, 158-171, 195-230, 254-267, 286-366, 390-457, and 481-487, and the motif for binding proteins, including G-proteins, at about amino acids 63-75 (See SEQ ID NO 100 and FIG. 22). The nucleic acids that code for BCU399 can be used for the generation of, e.g., nucleic acid probes, mutant sequences, including, e.g., chimeric sequences and antisense sequences, by PCR. BCU399 polypeptides and corresponding nucleic acids can be used, e.g., to generate antibodies for distinguishing between the human and monkey forms of BCU399. Its polypeptides and corresponding nucleic acids can be used to generate antibodies to the receptor surface to be used, e.g., as blocking agents in signal transduction pathways. The polypeptides or portions of them may be incorporated into resins for purification of ligands, e.g., G proteins, nucleotides, naturally-occurring ligands. The polypeptides can be used as competitors for ligand binding, e.g., ATP and G proteins, in ligand-binding assays.

BCU399 has several activities, including, e.g., nucleotide binding, ligand binding, signal transduction, phosphorylation, conformational change, etc. By “nucleotide binding” and “ligand binding” is meant the covalent or non-covalent association of a nucleotide, protein, or other molecule with one or more amino acids of BCU399, for example, as described in Merighi et al., British Journal of Pharmacology, 134:1215-26, 2001. By “signal transduction” is meant the activation of a chain of events that alters the concentration of one or more small intracellular signaling molecules (second messengers), e.g., cyclic AMP, calcium ions, as described in Sabala et al., British Journal of Pharmacology, 132:393-402, 2001. By “phosphorylation” is meant the covalent attachment to an amino acid, e.g., serine, threonine, tyrosine, etc., of a phosphate group from a nucleotide, e.g., ATP, GTP, etc., by means of a kinase, e.g., PK2, PKC, tyrosine kinase, etc. Hausdorff et al., FASEB Journal, 4:2881-2889, 1990. By “conformational change” is meant a change in the tertiary or quaternary structure of a polypeptide. Ballesteros et al., Molecular Pharmacology, 60:1-19, 2001. These activities can be determined routinely. For instance the binding affinity of nucleotides and other ligands can be measured with ligands fused to radioactive or fluorescent markers (e.g., γ³²P-ATP or green fluorescent protein) and visualized by phosphorimager analysis or fluorimetry. Signal transduction can be assessed by expression of BCU399 in cells, stimulation by appropriate ligands, e.g., nucleotides such as ATP, GTP, etc., or their analogs, and measurement of the concentrations of elicited second messengers or byproducts, e.g., Ca²⁺ or cAMP, by, e.g., atomic absorption spectrometry (ThermoElemental SOLAAR AA spectrometers), radioimmunoassay, etc. Phosphorylation can be assessed by, e.g., phosphorylation assay systems, (Perkin Elmer FlashPlate Plus). Conformational change can be assessed spectroscopically (circular dichroism, NMR spectroscopy) or using antibodies to specific conformations.

Human Kidins (PC473)

Human Kidins220Pc (kinase D-interacting substrate of 220 kDa) codes for a polypeptide containing 1715 amino acid. The nucleotide and amino acid sequences of Kidins220 are shown in SEQ ID NOS 88 and 89. It contains 11 ANK domains at about amino acid positions 37-66, 70-99, 103-132, 137-166, 170-199, 203-232, 236-265, 269-298, 302-331, 335-364, and 368-399. Four transmembrane domains are located at about amino acid positions 496-518, 525-547, 659-681, and 688-707. There is a SAM domain at about amino acids 1151-1223. It contains cAMP and cGMP protein linase phosphorylation site motifs at about 880-883, 901-904, 1250-1253, 1438-1441, and 1524-1527; protein kinase C phosphorylation site motifs at about 167-169, 219-221,233-235, 381-383, 471-473, 562-564, 590-592, 722-724, 791-793, 904-906, 939-941, 950-952, 998-1000, 1012-1014, 1034-1036, 1180-1182, 1298-1300, 1320-1322, 1351-1353, 1441-1443, 1567-1569, 1677-1679, and 1681-1683; ATP/GTP-binding site motif A (P-loop) at about amino acid positions 467-474; and tyrosine phosphorylation site motifs at 403-409 and 1397-1404. Its N- and C-terminus are cytoplasmic. A UniGene cluster is represented by Hs.9873.

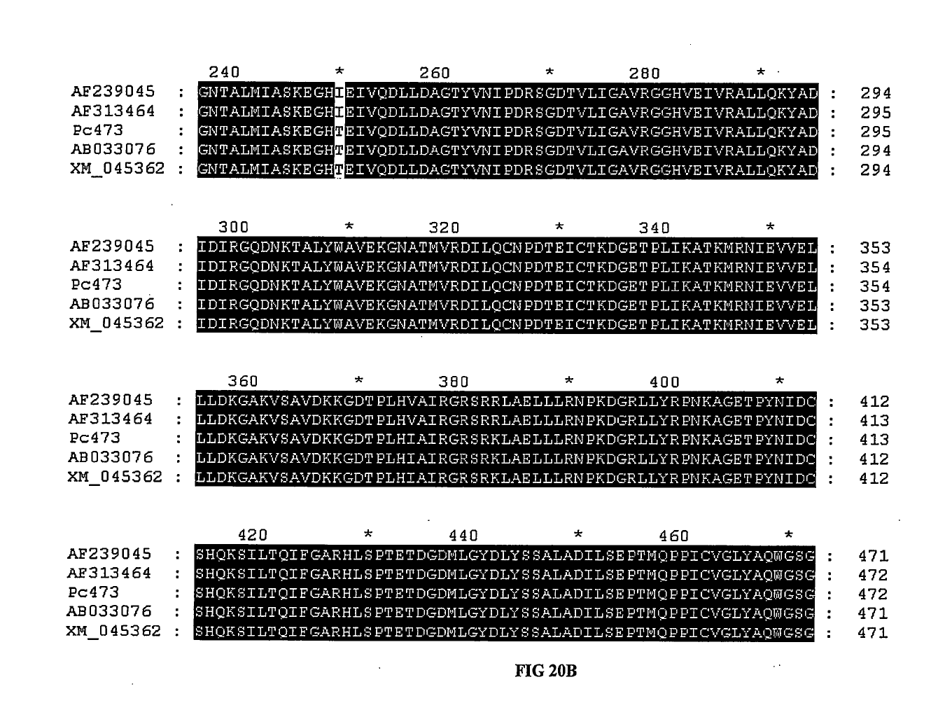

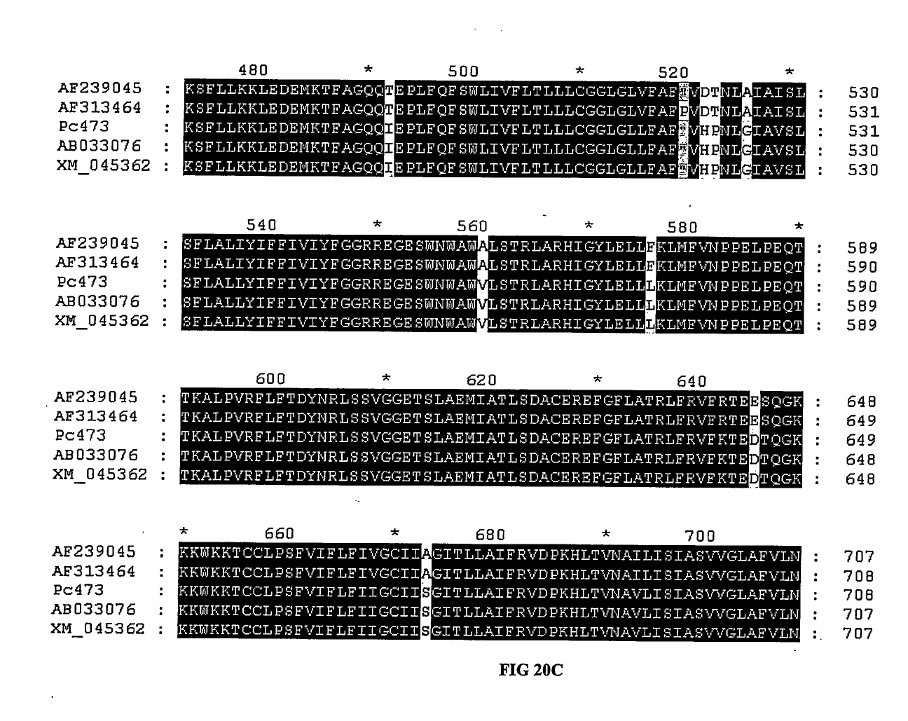

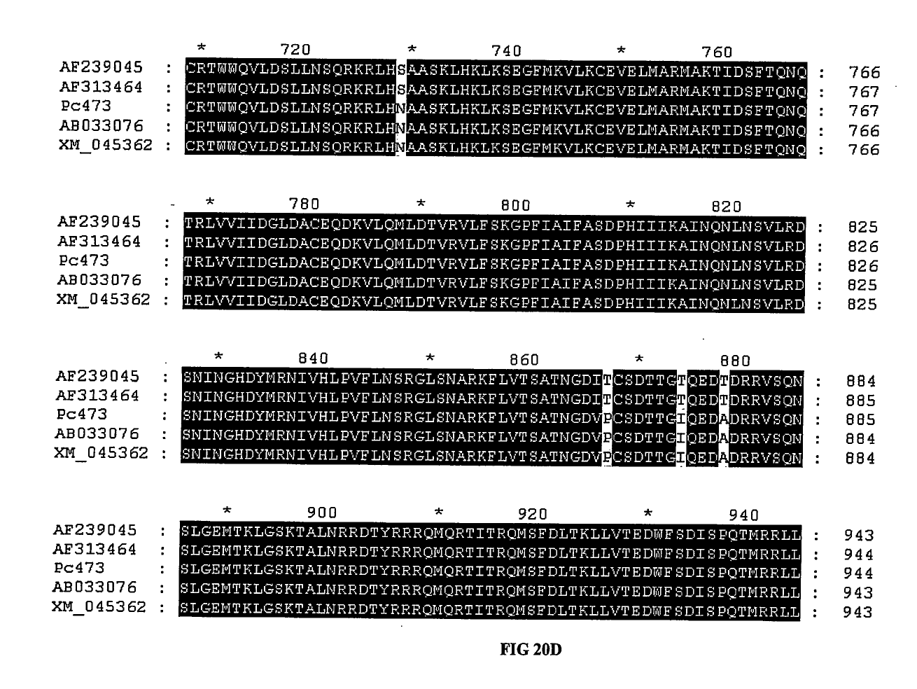

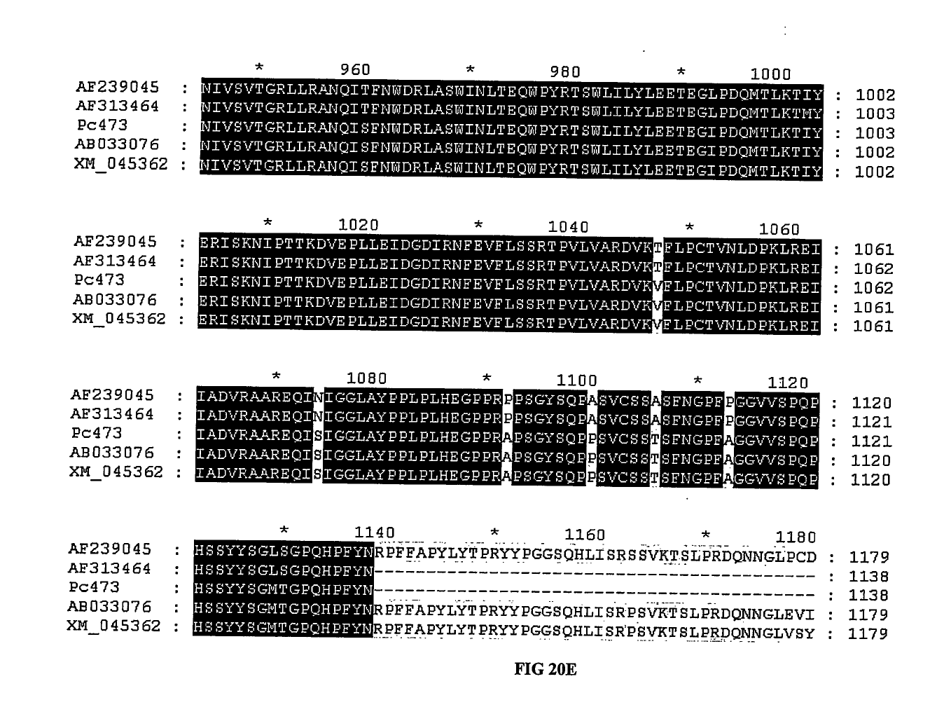

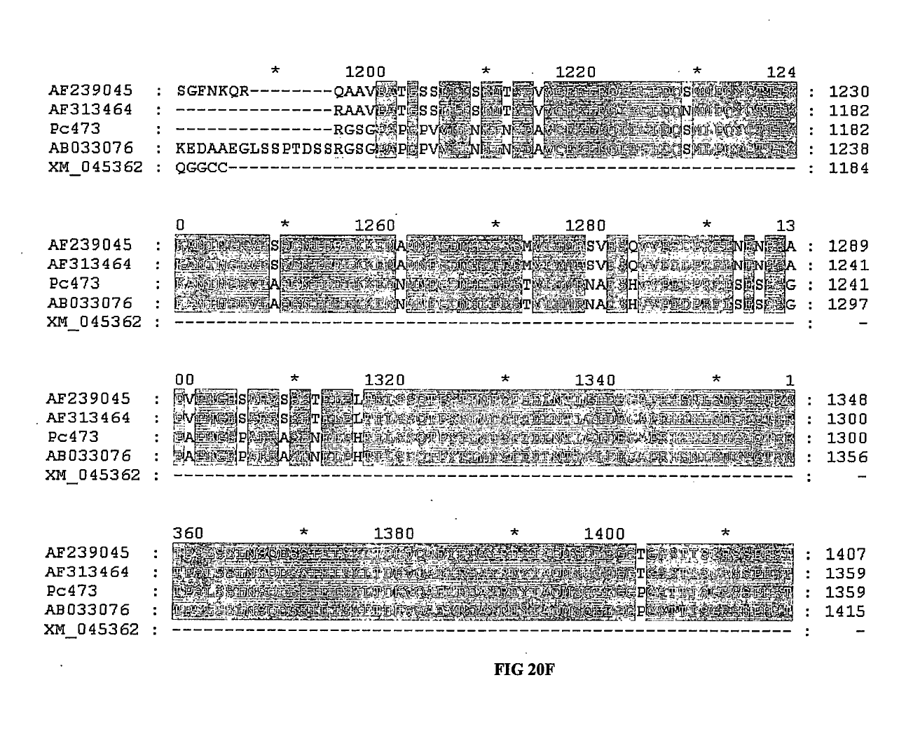

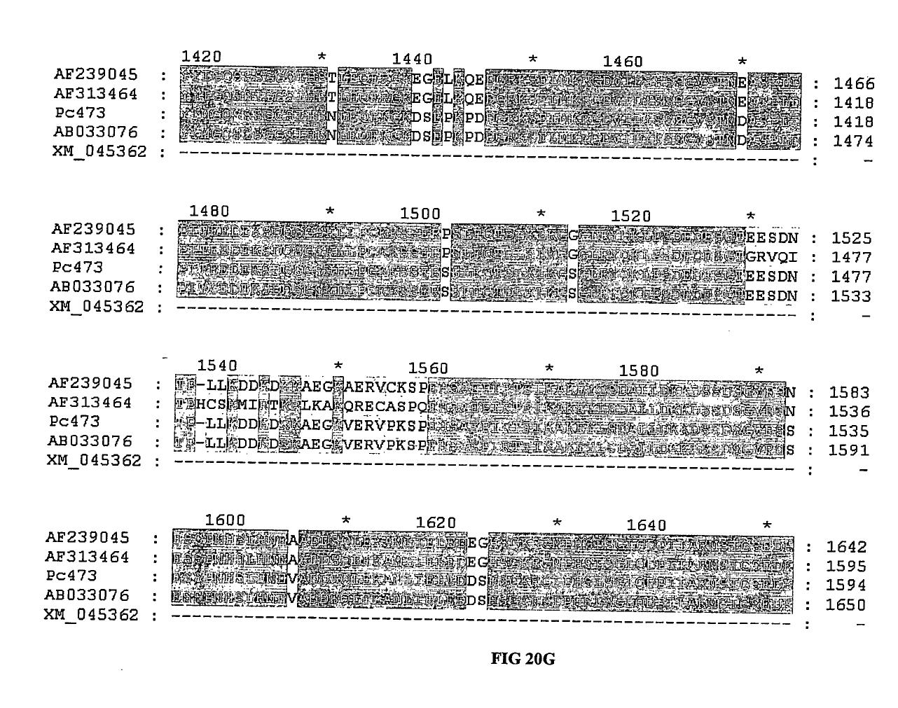

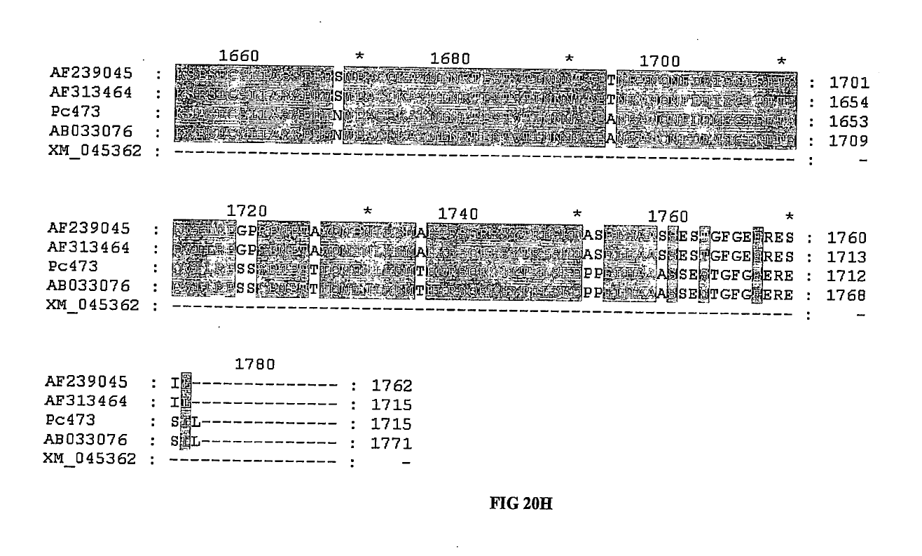

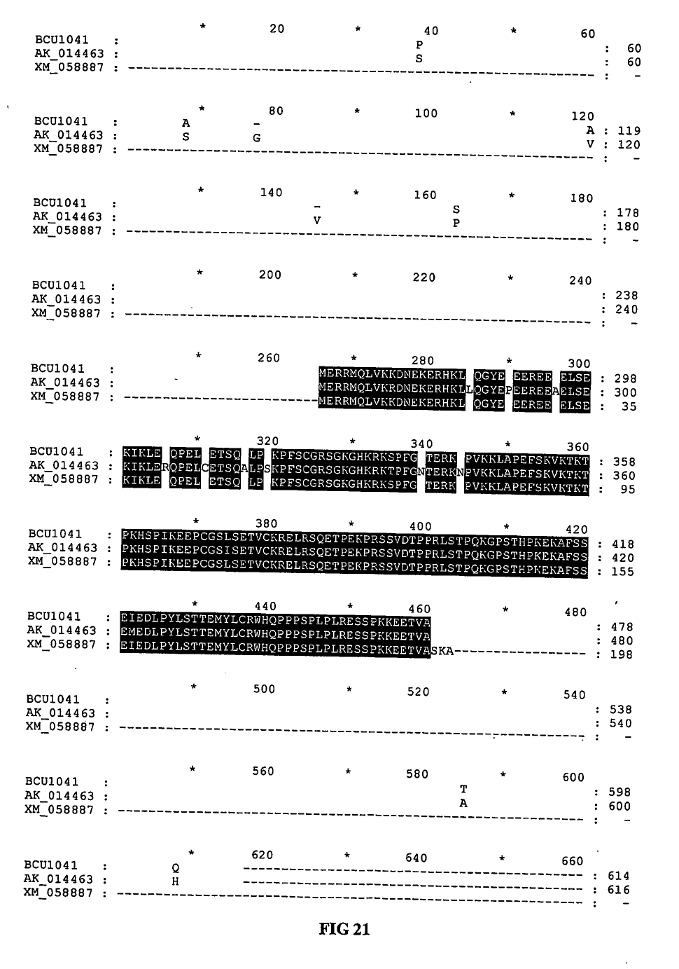

There are several alternative forms of Kidins220Pc (.e.g., different sequences as a result of alternative splicing, etc.). AB033076 (FIG. 20; SEQ ID NOS 91) appears to a complete cDNA having an insertion of about 57 amino acids after human Kidins220Pc residue 1138, as well as containing an addition amino acid residue, Q, at about amino acid position 136. See, FIG. 20. AB033076 also has a six-amino acid extension at its N-terminus, LQLSVK (SEQ ID NO 92), which is not shown. XM_{—}045362 (FIG. 20; SEQ ID NOS 90) is a partial and incomplete EST for human Kidins220Pc, missing from about amino acid 1138. See, FIG. 20. It contains the above-mentioned insertion, making it closer to the AB033076 variant. In addition to the Q residue at position 136, the following sequences (polypeptide and corresponding nucleotide) can be used to distinguish the different forms: 1138-1184 (SEQ ID NO 90), 1138-1176(SEQ ID NO 90), 1177-1184 (SEQ ID NO 90), 1138-1194 (SEQ ID NO 91), or 1177-1194 (SEQ ID NO 91).

There are several rat homologs of human Kidins220. AF313464 (FIG. 20; SEQ ID NO 93) shares about 92% amino acid sequence identity and 95% amino acid homology along its entire length. Like the human Kidins220Pcform, this rat homolog does not contain the amino acid insertion present in AB033076, but it does contain the Q residue at 136. AF239045 (FIG. 20; SEQ ID NO 94) is another rat homolog, closer to the AB033076 form, having about 91% amino acid sequence identity and 93% amino acid homology along its entire length to human kidins220Pc. A C. elegans homolog is NM-069656 and a Drosophila homolog is AE003453.

All or part-of Kidins220 is located in genomic DNA represented by GenBank ID: AC012495.8 and Contig ID: NT_{—}022194. The present invention relates to any isolated introns and exons that are present in the gene. Intron and exon boundaries can be routinely determined, e.g., using the polypeptide and genomic sequences disclosed herein.

Human Kidins220Pc maps to chromosomal band 2p25.1. Hereditary essential tremor (OMIM 602134) maps to this location. Nucleic acids of the present invention can be used as linkage markers, diagnostic targets, therapeutic targets, for this disorder, as well as any disorders or genes mapping in proximity to it.

Kidins220 was originally identified as a substrate protein kinase D (“PKD”), a serine/threonine kinase regulated by diacylglycerol signaling pathways. See, Iglesias, J. Biol. Chem., 275:40048-40056, 2000. It is phosphorylated by PKD at the serine at position 919, and its first physiologically-occurring substrate. See, Iglesias et al.. Thus, human Kidin220Pc can used as a substrate in assays for PKD activity. See, e.g. Iglesias et al. for how such assays can be carried out.

In addition to its association with prostate cancer, Kidins220Pc expression can be affected in other tissues, as well. For example, Iglesias et al. reported that it is expressed at very high levels in the brain and has a role in neurite ougriwth, making it useful for the treatment and analysis of neurodegenerative diseases, including spinal cord injuries, Parkinson's disease, Alzheimer's disease, multiple sclerosis, traumatic head injury, etc. For example, modulation of human kidins220Pc can be utilized to regulate neurite outgrowth and subsequent synaptogenesis.

DEPTA Genes

DEPTA-1, -2, and -3 (Pcp409, Pcp461, and Pcp578, respectively) are differentially expressed prostate tumor antigen genes (“DEPTA”) that are highly up-regulated in prostate cancers. DEPTA-1 (SEQ ID NO 84) and DEPTA-2 (SEQ ID NO 85) are non-coding transcripts. DEPTA-2 is encoded by three exons. DEPTA-1 is only a single exon and is located in the intron region of DEPTA-2. The present invention relates to the nucleic fragments comprising the individual introns and exons of the DEPTA-½ cluster. Expression of DEPTA-1 is highly restricted to the prostate, and substantially no other tissues. DEPTA-2 is not as highly restricted to prostate, but is expressed in testis and stomach tissue, as well.

DEPTA-3 codes for a polypeptide containing 139 amino acids. The nucleotide and amino acid sequences of DEPTA-3 are shown in SEQ ID NOS 86 and 87. DEPTA-3 is a highly-charged polypeptide having a putative phosphorylation site at about amino acid residues 49-51. It has homology to other proteins which have binding activity, suggesting that it binds to a nucleic acid or protein binding partner. DEPTA-3 is expressed in normal prostate, as well as kidney, heart, stomach, pancreas, and thyroid.

All or part of DEPTA-3 is located in genomic DNA represented by GenBank ID: AC018601, BAC-ID: RP11-28G15, and Contig ID: NT_{—}0054207. Its 5′ end is associated with UniGene cluster Hs.135941. The present invention relates to any isolated introns and exons that are present in the gene. Intron and exon boundaries can be routinely determined, e.g., using the polypeptide and genomic sequences disclosed herein. DEPTA-3 maps to chromosomal band 2p13.

Other Genes

Membrane (i.e., cell-surface) proteins coded for by up-regulated genes (e.g., PCP0816) are useful targets for antibodies and other binding partners (e.g., ligands, aptamers, small peptides, etc.) to selectively target agents to a breast cancer tissue for any purpose, included, but not limited to, imaging, therapeutic, diagnostic, drug delivery, gene therapy, etc. For example, binding partners, such as antibodies, can be used to treat carcinomas in analogy to how c-erbB-2 antibodies are used to breast cancer. Membrane (e.g., PCP0405 when shed into the blood and other fluid) and extracellular proteins (e.g., PCP0389 or PCP0664) can also be used as diagnostic markers for cancer, and to assess the progress of the disease, e.g., in analogy to how PSA levels are used to diagnose prostate cancer. Useful antibodies or other binding partners include those that are specific for parts of the polypeptide which are exposed extracellularly as indicated in Table 1 and 4. Tables 3 and 4 summarize the expression profile of these genes.

Polynucleotides of the present invention can also be used to detect metastatic cells in the blood. For instance, PCP0389, PCP0814, PCP0424, PC0382, PCP0840, PCP0842, PCP0405, PC0177, PCP0677, and PCP0806 are absent from peripheral blood cells, and can therefore be used in diagnostic tests to assess whether prostate cancer cells have metastasized from the primary site.

Polynucleotides of the present invention have been mapped to specific chromosomal bands. Different human disorders are associated with these chromosome locations. See, Tables 2 and 5. The polynucleotides and polypeptides they encode can be used as linkage markers, diagnostic targets, therapeutic targets, for any of the mentioned disorders, as well as any disorders or genes mapping in proximity to them. Of particular interest are those genes which map to cancer loci, such as PCP0749, PCP0814, PCP0816, PCP0405, PCP0459, PCP0677, and PCP0762.

The present invention relates to the complete polynucleotide and polypeptide sequences disclosed herein, as well as fragments thereof Useful fragments include those which are unique and which do not overlap any known gene (e.g., amino acid residues 1-394 of SEQ ID NO 2 of PCP0749), which overlap with a known sequence (e.g., amino acids residues 395-1564 of SEQ ID NO 2 of PCP0749, which span alternative splice junctions (e.g., comprising amino acid residues 585-586 of PCP0424A of SEQ ID NO 18), which are unique to a public sequence as indicated in the figures (e.g., e.g., amino acids residues 2149-2265 of NM_{—}133433 of SEQ ID NO 35), which span an alternative splice junction of a public sequence (e.g., 532-533 of NM_{—}005690 of SEQ ID NO 40), etc. Unique sequences can also be described as being specific for a gene because they are characteristic of the gene, but not related genes. The unique or specific sequences included polypeptide sequences, coding nucleotide sequences (e.g., as illustrated in the figures), and non-coding nucleotide sequences.

Below, for illustration, are some examples of polypeptides (included are the polynucleotides which encode them); however, the present invention includes all fragments, especially of the categories mentioned above are exemplified below.

PCP0749 (SEQ ID NO 1-2):polypeptides comprising, consisting of, or consisting essentially of about amino acids 1-394, polypeptide fragments thereof, and polynucleotides encoding said polypeptides;

PCP0389 (SEQ ID NO 5-6): polypeptides comprising, consisting of, or consisting essentially of about amino acids 1-1-117, polypeptide fragments thereof, and polynucleotides encoding said polypeptides;

PCP0814 (SEQ ID NO 9-10): polypeptides comprising, consisting of, or consisting essentially of about amino acids 1-33, polypeptide fragments thereof, and polynucleotides encoding said polypeptides;

PCP0623 (SEQ ID NO 11-12): polypeptides comprising, consisting of, or consisting essentially of about amino acids 1-539, polypeptide fragments thereof, and polynucleotides encoding said polypeptides;

PCP0815 (SEQ ID NO 13-14): polypeptides comprising, consisting of, or consisting essentially of about amino acids 1-22, 964-1010, 1011-1041, polypeptide fragments thereof, and polynucleotides encoding said polypeptides;

PCP0840 (SEQ ID NO 15-16): polypeptides comprising, consisting of, or consisting essentially of about amino acids 1-129, polypeptide fragments thereof, and polynucleotides encoding said polypeptides;

PCP0424A (SEQ ID NO 17-18): polypeptides comprising, consisting of, or consisting essentially of about amino acids 1-53, 585-586, 586-611, polypeptide fragments thereof, and polynucleotides encoding said polypeptides;

PCP0424B (SEQ ID NO 19-20): polypeptides comprising, consisting of, or consisting essentially of about amino acids 1-53, 585-586, polypeptide fragments thereof, and polynucleotides encoding said polypeptides;

PCP0424C (SEQ ID NO 21-22): polypeptides comprising, consisting of, or consisting essentially of about amino acids 1-53, 585-586, polypeptide fragments thereof, and polynucleotides encoding said polypeptides;

PCP0816 (SEQ ID NO 25-26): polypeptides comprising, consisting of; or consisting essentially of about amino acids 268-317, 623, 992-1013, polypeptide fragments thereof, and polynucleotides encoding said polypeptides;

PCP0480 (SEQ ID NO 27-28): polypeptides comprising, consisting of; or consisting essentially of about amino acids 1-151, 152-171, polypeptide fragments thereof, and polynucleotides encoding said polypeptides;

PC0382 (SEQ ID NO 23-24): polypeptides comprising, consisting of, or consisting essentially of about amino acids 1-9, polypeptide fragments thereof, and polynucleotides encoding said polypeptides;

PCP0842 (SEQ ID NO 29-30): polypeptides comprising, consisting of, or consisting essentially of about amino acids 1-454, polypeptide fragments thereof, and polynucleotides encoding said polypeptides.

PCP405 (SEQ ID NO 45-46): polypeptides comprising, consisting of, or consisting essentially of about amino acids 1-351, 941, polypeptide fragments thereof, and polynucleotides encoding said polypeptides. PCP405 has high expression in the adrenal gland, brain and pituitary gland, and codes for a polypeptide which comprises domains characteristic of the attractins and other cell adhesion and guidance proteins. See, e.g., Duke-Cohan et al., Proc. Natl. Acad. Sci., 95:11336-11341, 1998.

PC0177A (SEQ ID NOS 54-55): polypeptides comprising, consisting of; or consisting essentially of about amino acids 1-85, 560-594, 1139-1167, 1167-1168, 1168-1744, polypeptide fragments thereof; and polynucleotides encoding said polypeptides; PC0177B (SEQ ID NOS 56-57): polypeptides comprising, consisting of, or consisting essentially of about amino acids 1-85, 559-560, 1104-1132, 1132-1133, 1132-1709, polypeptide fragments thereof, and polynucleotides encoding said polypeptides; PC0177C (SEQ ID NOS 58-59): polypeptides comprising, consisting of, or consisting essentially of about amino acids 1-85, 559-560, 11041132, 1703-1908, polypeptide fragments thereof, and polynucleotides encoding said polypeptides; PC0177D (SEQ ID NOS 60-61): polypeptides comprising, consisting of, or consisting essentially of about amino acids 1-85, 559-560, polypeptide fragments thereof, and polynucleotides encoding said polypeptides. PC0177 comprise coil-coil domains involved in protein interactions.

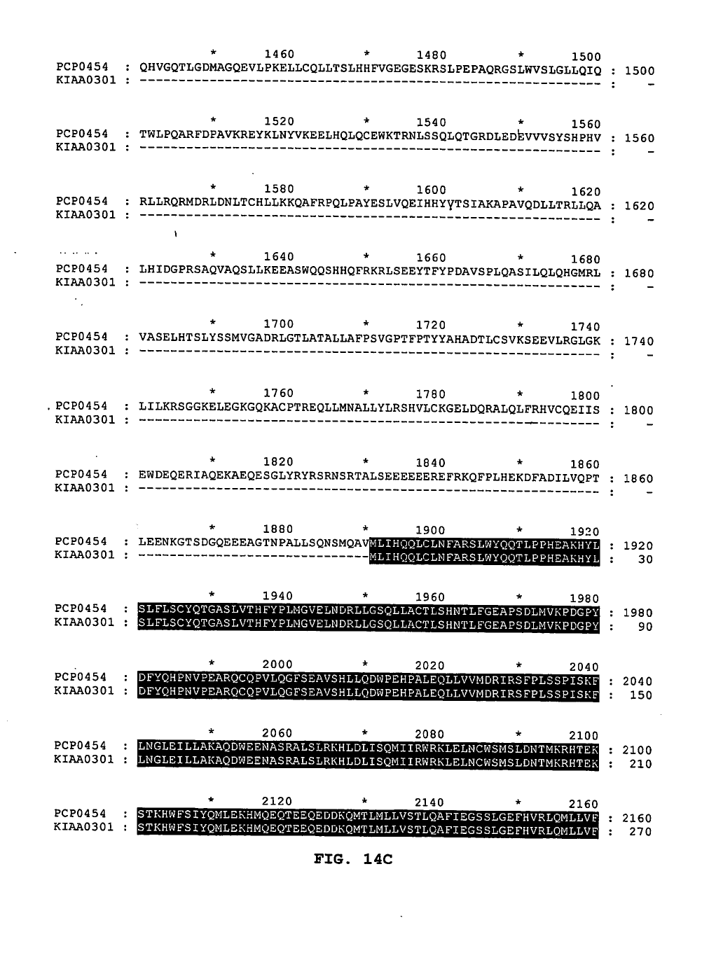

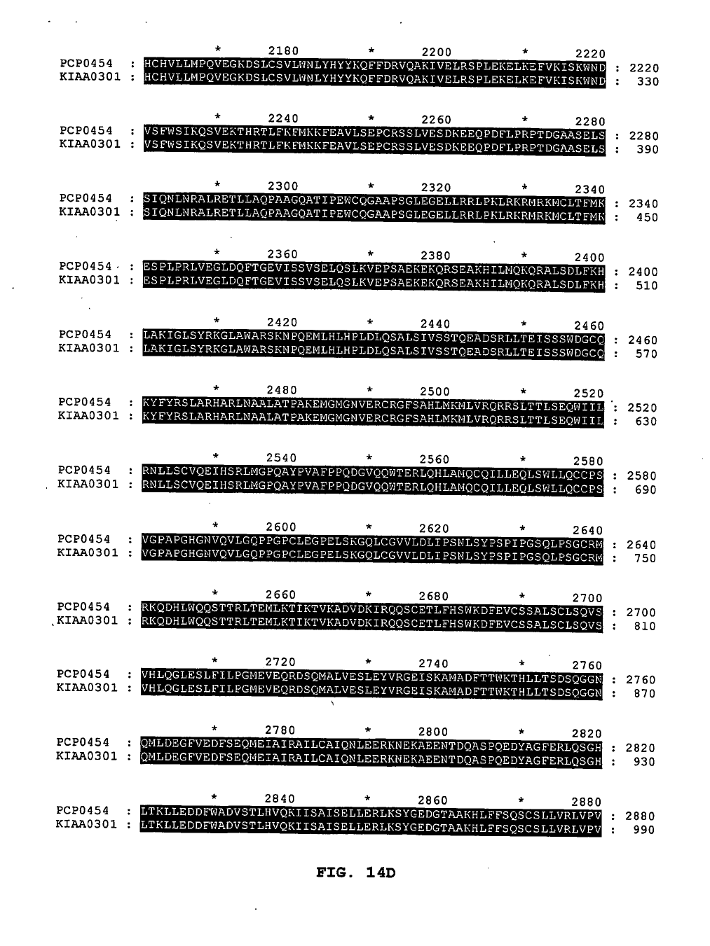

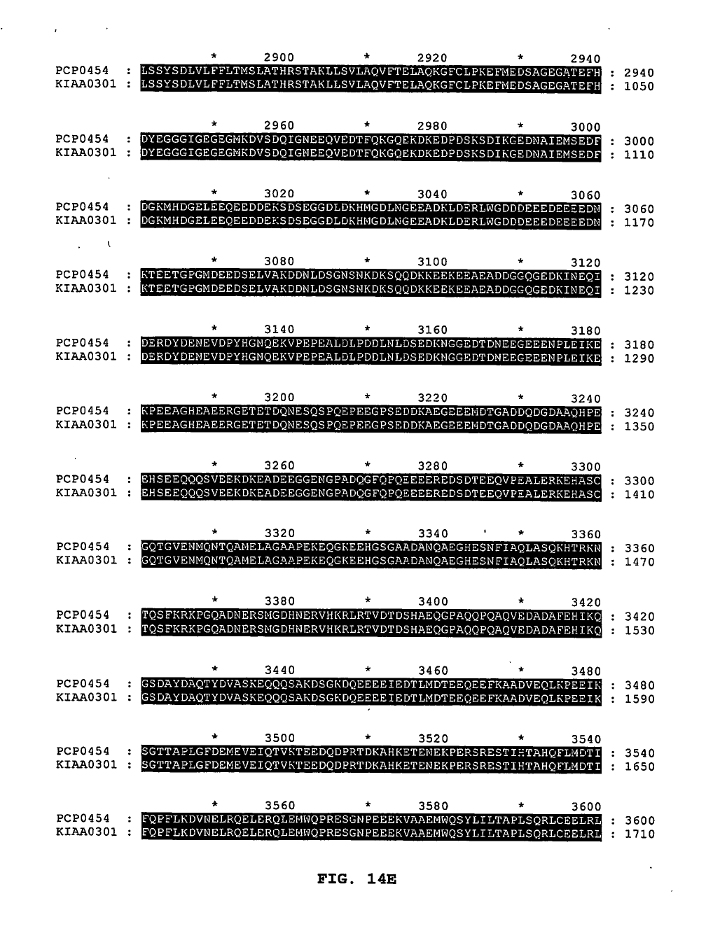

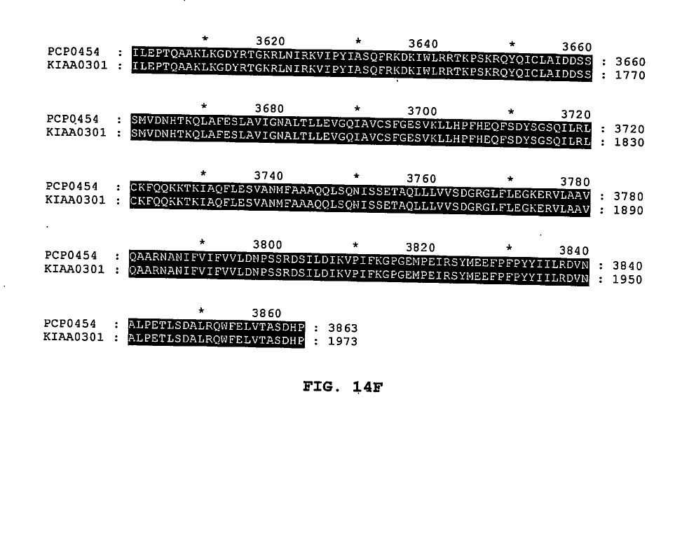

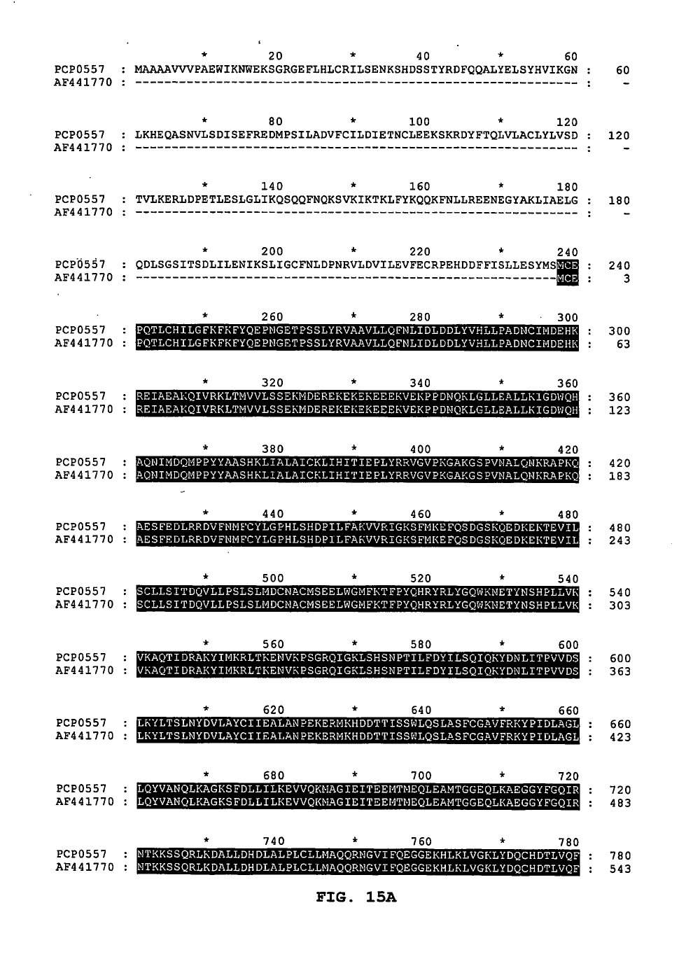

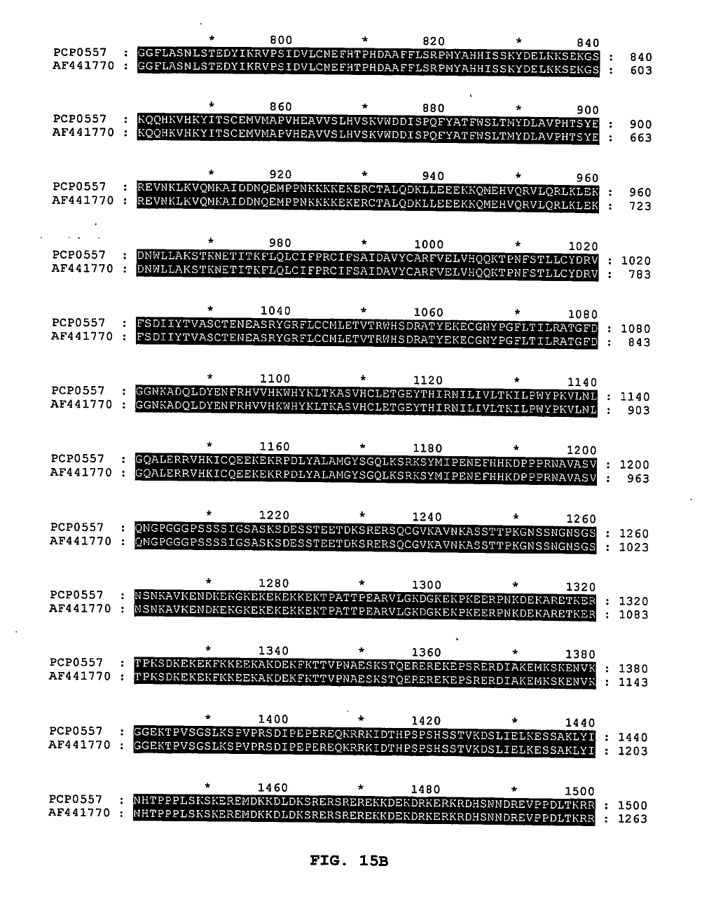

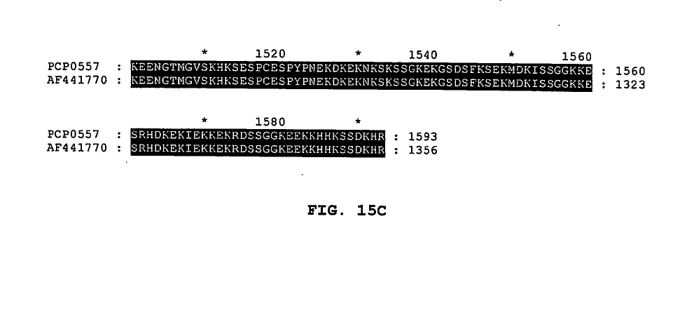

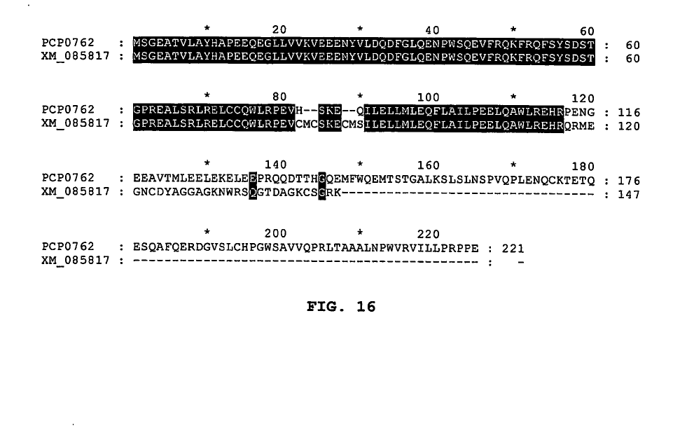

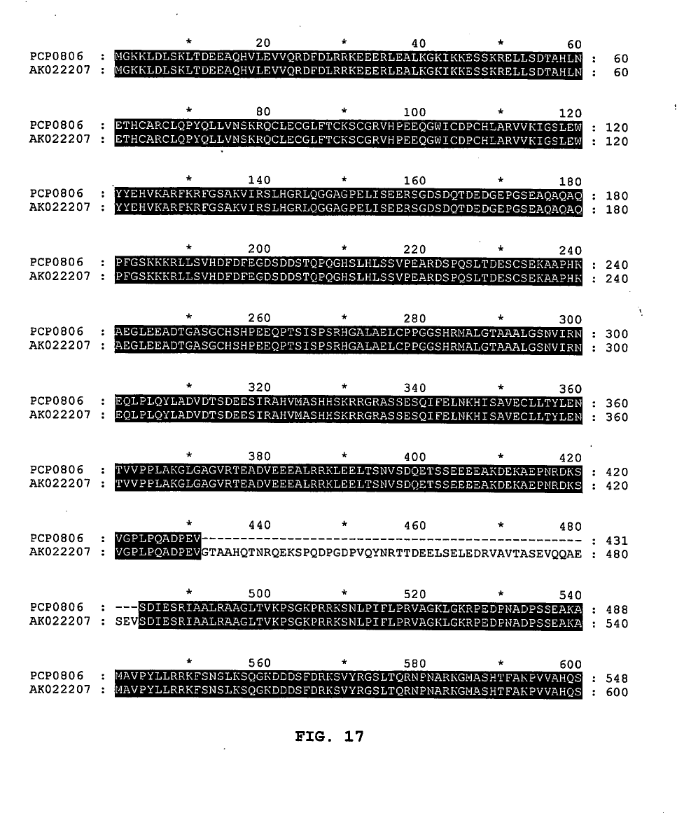

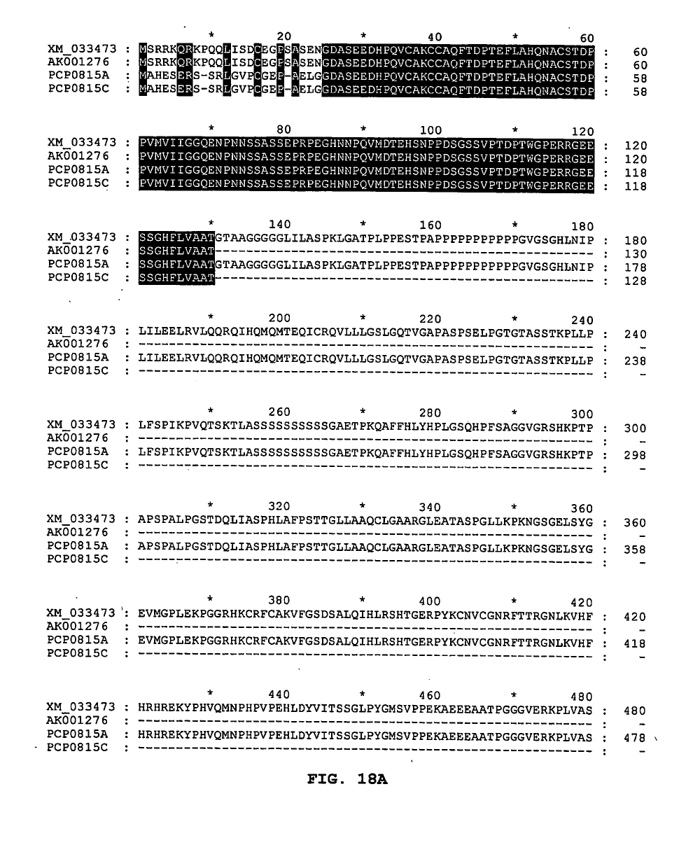

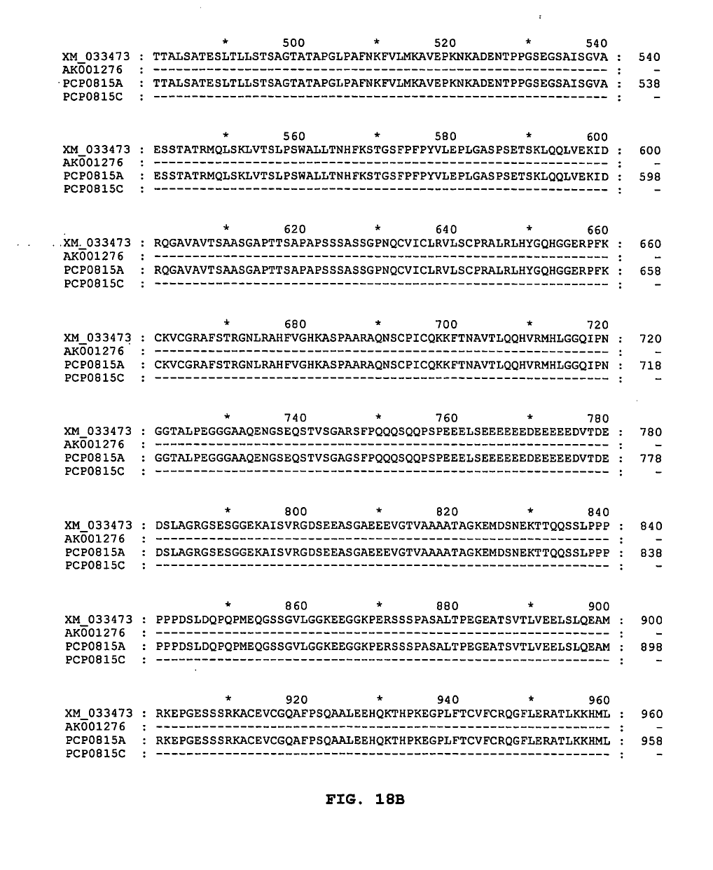

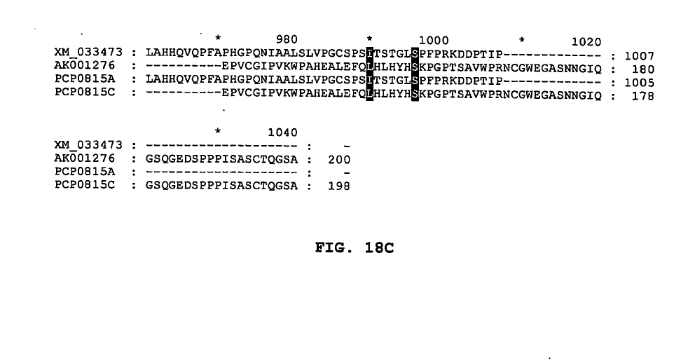

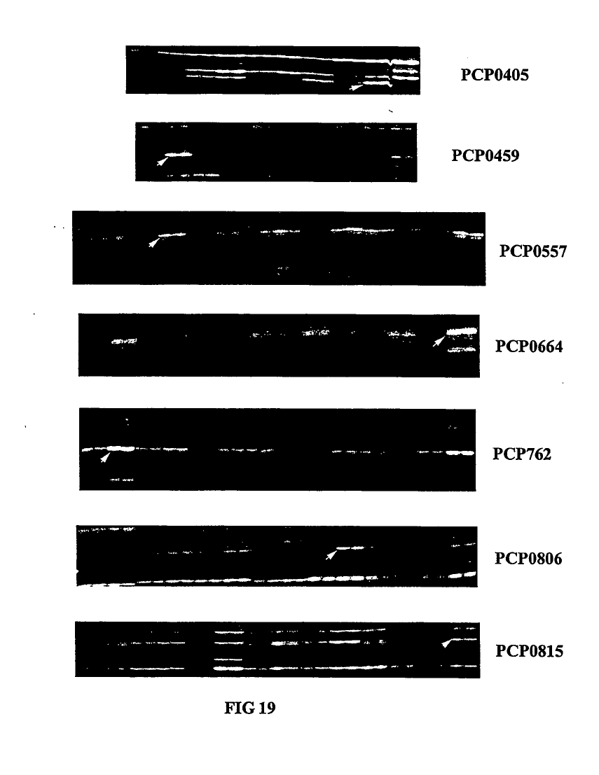

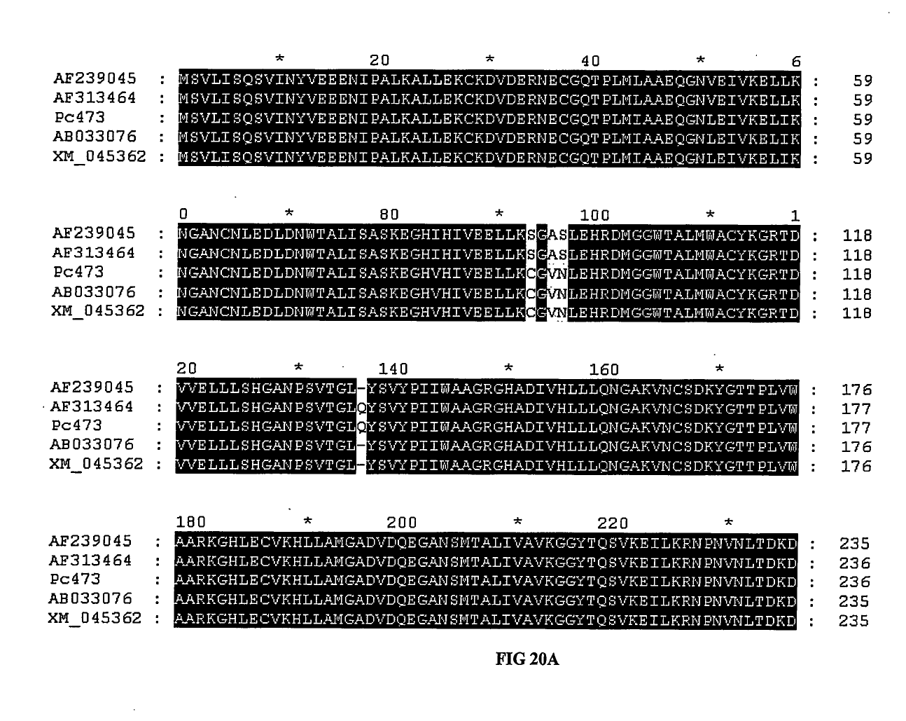

PCP454A (SEQ ID NO 50-51; FIG. 14): polypeptides comprising, consisting of, or consisting essentially of about amino acids 1-1890, polypeptide fragments thereof, and polynucleotides encoding said polypeptides. PCP454B (SEQ ID NOS 48-49) codes for a 577-amino acid polypeptide. This polypeptide comprises a nucleotide binding site which can be used to assay for its activity, e.g., by a filtration-type assay using radioactive ATP or other nucleotides. Nucleotide binding can also be used to purify the polypeptide, e.g., using a column comprising a nucleotide. PCP454A and B are contiguous, and a transcript has also been detected (SEQ ID NO 47) which comprises both open reading frames, where 454B is in the 5′ region, and about 2 kb down from it is 454A.

PCP0557 (SEQ ID 62-63): polypeptides comprising, consisting of, or consisting essentially of about amino acids 1-237, polypeptide fragments thereof, and polynucleotides encoding said polypeptides. PCP0557 polypeptide has a phosphoacceptor domain indicating that it is involved in signal transduction. This domain (e.g., amino acids 565-620) can be used as a substrate in kinase assays, e.g., as described in Kemp et al., “Design and use of peptide substrates for protein kinases,” Methods in Enzymol., 200:121-34, 1991; Wang et al., “Identification of the major site of rat prolactin phosphorylation as serine 177,” J. Biol. Chem., 271:2462-9, 1996; Yasuda et al., “A synthetic peptide substrate for selective assay of protein kinase C,” Biochem. Biophys. Res. Comm., 166:1220-7, 1990; Gonzalez et al., “Use of the synthetic peptide neurogranin(28-43) as a selective protein kinase C substrate in assays of tissue homogenates,” Anal. Biochem., 215:184-9, 1993; Parker et al., “Development of high throughput screening assays using fluorescence polarization: nuclear receptor-ligand-binding and kinase/phosphatase assays,” J. Biomol. Screen., 5:77-88, April 2000. See, also., U.S. Pat. Nos. 6,203,994, 6,074,861, 6,066,462, 6,004,757, and 5,741,689.

PCP0762 (SEQ ID NO 68-69): polypeptides comprising, consisting of, or consisting essentially of about amino acids 82-86, 113-221, polypeptide fragments thereof, and polynucleotides encoding said polypeptides. It contains a SCAN domain involved in transcriptional regulation.

PCP0806 (SEQ ID NO 70-71): polypeptides comprising, consisting of, or consisting essentially of about amino acids 31-32, polypeptide fragments thereof, and polynucleotides encoding said polypeptides. PC0806 is in an intracellular protein that shows high expression in lung, pancreas, prostate, and stomach.

PCP08 15A (SEQ ID NO 72-73): polypeptides comprising, consisting of, or consisting essentially of about amino acids 1-24, 131-1005, 744, polypeptide fragments thereof, and polynucleotides encoding said polypeptides; PCP0815C (SEQ ID NO 74-75): polypeptides comprising, consisting of, or consisting essentially of about amino acids 1-24, polypeptide fragments thereof, and polynucleotides encoding said polypeptides. The gene is expressed in many tissues, but is highest in brain and pituitary. PCP0815A comprises seven zinc finger domains, indicating that it is a transcriptional regulator. PCP0815C is missing these transcriptional domains, indicating that it can be a regulator (e.g., a negative regulator) of PCP0815A.

PCP0664 (SEQ ID NO 64-65) is a 122 amino acid polypeptide comprising an N-terminal hydrophobic region. It has a signal peptide cleavage site at about between amino acids 18 and 19, indicating that it can be a secreted molecule.

Nucleic Acids

A mammalian polynucleotide, or fragment thereof, of the present invention is a polynucleotide having a nucleotide sequence obtainable from a natural source. When the species name is used, e.g., a human, it indicates that the polynucleotide or polypeptide is obtainable from a natural source. It therefore includes naturally-occurring normal, naturally-occurring mutant, and naturally-occurring polymorphic alleles (e.g., SNPs), differentially-spliced transcripts, splice-variants, etc. By the term “naturally-occurring,” it is meant that the polynucleotide is obtainable from a natural source, e.g., animal tissue and cells, body fluids, tissue culture cells, forensic samples. Natural sources include, e.g., living cells obtained from tissues and whole organisms, tumors, cultured cell lines, including primary and immortalized cell lines. Naturally-occurring mutations can include deletions (e.g., a truncated amino- or carboxy-terminus), substitutions, inversions, or additions of nucleotide sequence. These genes can be detected and isolated by polynucleotide hybridization according to methods which one skilled in the art would know, e.g., as discussed below.

A polynucleotide according to the present invention can be obtained from a variety of different sources. It can be obtained from DNA or RNA, such as polyadenylated mRNA or total RNA, e.g., isolated from tissues, cells, or whole organism. The polynucleotide can be obtained directly from DNA or RNA, from a cDNA library, from a genomic library, etc. The polynucleotide can be obtained from a cell or tissue (e.g., from an embryonic or adult tissues) at a particular stage of development, having a desired genotype, phenotype, disease status, etc. A polynucleotide which “codes without interruption” refers to a polynucleotide having a continuous open reading frame (“ORF”) as compared to an ORF which is interrupted by introns or other noncoding sequences.

Polynucleotides and polypeptides (including any part of a differentially regulated cancer gene) can be excluded as compositions from the present invention if, e.g., listed in a publicly available databases on the day this application was filed and/or disclosed in a patent application having an earlier filing or priority date than this application and/or conceived and/or reduced to practice earlier than a polynucleotide in this application.

As described herein, the phrase “an isolated polynucleotide which is SEQ ID NO,” or “an isolated polynucleotide which is selected from SEQ ID NO,” refers to an isolated nucleic acid molecule from which the recited sequence was derived (e.g., a cDNA derived from mRNA; cDNA derived from genomic DNA). Because of sequencing errors, typographical errors, etc., the actual naturally-occurring sequence may differ from a SEQ ID listed herein. Thus, the phrase indicates the specific molecule from which the sequence was derived, rather than a molecule having that exact recited nucleotide sequence, analogously to how a culture depository number refers to a specific cloned fragment in a cryotube.

As explained in more detail below, a polynucleotide sequence of the invention can contain the complete sequence as shown in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72,74, 84, 85, 86, 88, 95, and/or 99, degenerate sequences thereof, anti-sense, muteins thereof, genes comprising said sequences, full-length cDNAs comprising said sequences, complete genomic sequences, fragments thereof, homologs, primers, nucleic acid molecules which hybridize thereto, derivatives thereof, etc.

Genomic

The present invention also relates genomic DNA from which the polynucleotides of the present invention can be derived. A genomic DNA coding for a human, mouse, or other mammalian polynucleotide, can be obtained routinely, for example, by screening a genomic library (e.g., a YAC library) with a polynucleotide of the present invention, or by searching nucleotide databases, such as GenBank and EMBL, for matches. Promoter and other regulatory regions (including both 5′ and 3′ regions, as well introns) can be identified upstream or downstream of coding and expressed RNAs, and assayed routinely for activity, e.g., by joining to a reporter gene (e.g., CAT, GFP, alkaline phosphatase, luciferase, galatosidase). A promoter obtained from a differentially regulated cancer gene can be used, e.g., in gene therapy to obtain tissue-specific expression of a heterologous gene (e.g., coding for a therapeutic product or cytotoxin). 5′ and 3′ sequences (including, UTRs and introns) can be used to modulate or regulate stability, transcription, and translation of nucleic acids, including the sequence to which is attached in nature, as well as heterologous nucleic acids.

Constructs

A polynucleotide of the present invention can comprise additional polynucleotide sequences, e.g., sequences to enhance expression, detection, uptake, cataloging, tagging, etc. A polynucleotide can include only coding sequence; a coding sequence and additional non-naturally occurring or heterologous coding sequence (e.g., sequences coding for leader, signal, secretory, targeting, enzymatic, fluorescent, antibiotic resistance, and other functional or diagnostic peptides); coding sequences and non-coding sequences, e.g., untranslated sequences at either a 5′ or 3′ end, or dispersed in the coding sequence, e.g., introns.

A polynucleotide according to the present invention also can comprise an expression control sequence operably linked to a polynucleotide as described above. The phrase “expression control sequence” means a polynucleotide sequence that regulates expression of a polypeptide coded for by a polynucleotide to which it is functionally (“operably”) linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can include an initiation codon and additional nucleotides to place a partial nucleotide sequence of the present invention in-frame in order to produce a polypeptide (e.g., pET vectors from Promega have been designed to permit a molecule to be inserted into all three reading frames to identify the one that results in polypeptide expression). Expression control sequences can be heterologous or endogenous to the normal gene.

A polynucleotide of the present invention can also comprise nucleic acid vector sequences, e.g., for cloning, expression, amplification, selection, etc. Any effective vector can be used. A vector is, e.g., a polynucleotide molecule which can replicate autonomously in a host cell, e.g., containing an origin of replication. Vectors can be useful to perform manipulations, to propagate, and/or obtain large quantities of the recombinant molecule in a desired host. A skilled worker can select a vector depending on the purpose desired, e.g., to propagate the recombinant molecule in bacteria, yeast, insect, or mammalian cells. The following vectors are provided by way of example. Bacterial: pQE70, pQE60 pQE-9 (Qiagen), pBS, pD10, Phagescript, phiX174, pBK Phagemid, pNH8A, pNH16a, pNH18Z, pNH46A (Stratagene); Bluescript KS+II (Stratagene); ptrc99a, pKK223-3, pKK233-3, pDR54 0, pRIT5 (Pharmacia). Eukaryotic: PWLNEO, pSV2CAT, pOG44, pXT1, pSG (Stratagene), pSVK3, PBPV, PMSG, pSVL (Pharmacia), pCR2.1/TOPO, pCRII/TOPO, pCR4/TOPO, pTrcHisB, pCMV6-XLA, etc. However, any other vector, e.g., plasmids, viruses, or parts thereof, may be used as long as they are replicable and viable in the desired host. The vector can also comprise sequences which enable it to replicate in the host whose genome is to be modified.

Hybridization

Polynucleotide hybridization, as discussed in more detail below, is useful in a variety of applications, including, in gene detection methods, for identifying mutations, for making mutations, to identify homologs in the same and different species, to identify related members of the same gene family, in diagnostic and prognostic assays, in therapeutic applications (e.g., where an antisense polynucleotide is used to inhibit expression), etc.

The ability of two single-stranded polynucleotide preparations to hybridize together is a measure of their nucleotide sequence complementarity, e.g., base-pairing between nucleotides, such as A-T, G-C, etc. The invention thus also relates to polynucleotides, and their complements, which hybridize to a polynucleotide comprising a nucleotide sequence as set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 and genomic sequences thereof. A nucleotide sequence hybridizing to the latter sequence will have a complementary polynucleotide strand, or act as a template for one in the presence of a polymerase (i.e., an appropriate polynucleotide synthesizing enzyme). The present invention includes both strands of polynucleotide, e.g., a sense strand and an anti-sense strand.

Hybridization conditions can be chosen to select polynucleotides which have a desired amount of nucleotide complementarity with the nucleotide sequences set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 and genomic sequences thereof. A polynucleotide capable of hybridizing to such sequence, preferably, possesses, e.g., about 70%, 75%, 80%, 85%, 87%, 90%, 92%, 95%, 97%, 99%, or 100% complementarity, between the sequences. The present invention particularly relates to polynucleotide sequences which hybridize to the nucleotide sequences set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 or genomic sequences thereof, under low or high stringency conditions. These conditions can be used, e.g., to select corresponding homologs in non-human species.

Polynucleotides which hybridize to polynucleotides of the present invention can be selected in various ways. Filter-type blots (i.e., matrices containing polynucleotide, such as nitrocellulose), glass chips, and other matrices and substrates comprising polynucleotides (short or long) of interest, can be incubated in a prehybridization solution (e.g., 6×SSC, 0.5% SDS, 100 μg/ml denatured salmon sperm DNA, 5× Denhardt's solution, and 50% formamide), at 22-68° C., overnight, and then hybridized with a detectable polynucleotide probe under conditions appropriate to achieve the desired stringency. In general, when high homology or sequence identity is desired, a high temperature can be used (e.g., 65 ° C.). As the homology drops, lower washing temperatures are used. For salt concentrations, the lower the salt concentration, the higher the stringency. The length of the probe is another consideration. Very short probes (e.g., less than 100 base pairs) are washed at lower temperatures, even if the homology is high. With short probes, formainde can be omitted. See, e.g., Current Protocols in Molecular Biology, Chapter 6, Screening of Recombinant Libraries; Sambrook et al., Molecular Cloning, 1989, Chapter 9.

For instance, high stringency conditions can be achieved by incubating the blot overnight (e.g., at least 12 hours) with a polynucleotide probe in a hybridization solution containing, e.g., about 5×SSC, 0.5% SDS, 100 μg/ml denatured salmon sperm DNA and 50% formamide, at 42° C., or hybridizing at 42° C. in 5×SSPE, 0.5% SDS, and 50% formamide, 100 μg/ml denatured salmon sperm DNA, and washing at 65° C. in 0.1% SSC and 0.1% SDS.

Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity.

Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for, e.g., less than 10%, less than 5% mismatch, etc., reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm =(number of A's and T's)×2° C.+(number of C's and G's)×4° C. For longer molecules, Tm=81.5+16.6 log₁₀[Na⁺]+0.41(% GC)−600/N where [Na⁺] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 90%, 95%, 97%, or more, etc., nucleotide complementarity between the probe (e.g., a short polynucleotide of SEQ ED NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 or genomic sequences thereof) and a target polynucleotide.

Other homologs of polynucleotides of the present invention can be obtained from mammalian and non-mammalian sources according to various methods. For example, hybridization with a polynucleotide can be employed to select homologs, e.g., as described in Sambrook et al., Molecular Cloning, Chapter 11, 1989. Such homologs can have varying amounts of nucleotide and amino acid sequence identity and similarity to such polynucleotides of the present invention. Mammalian organisms include, e.g., mice, rats, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, artemia, viruses, etc. The degree of nucleotide sequence identity between human and mouse can be about, e.g. 70% or more, 85% or more for open reading frames, etc.

Alignment

Alignments can be accomplished by using any effective algorithm. For pairwise alignments of DNA sequences, the methods described by Wilbur-Lipman (e.g., Wilbur and Lipman, Proc. Natl. Acad Sci., 80:726-730, 1983) or Martinez/Needleman-Wunsch (e.g., Martinez, Nucleic Acid Res., 11:4629-4634, 1983) can be used. For instance, if the Martinez/Needleman-Wunsch DNA alignment is applied, the minimum match can be set at 9, gap penalty at 1.10, and gap length penalty at 0.33. The results can be calculated as a similarity index, equal to the sum of the matching residues divided by the sum of all residues and gap characters, and then multiplied by 100 to express as a percent. Similarity index for related genes at the nucleotide level in accordance with the present invention can be greater than 70%, 80%, 85%, 90%, 95%, 99%, or more. Pairs of protein sequences can be aligned by the Lipman-Pearson method (e.g., Lipman and Pearson, Science, 227:1435-1441, 1985) with k-tuple set at 2, gap penalty set at 4, and gap length penalty set at 12. Results can be expressed as percent similarity index, where related genes at the amino acid level in accordance with the present invention can be greater than 65%, 70%, 75%, 80%, 85%, 90%, 95%, 99%, or more. Various commercial and free sources of alignment programs are available, e.g., MegAlign by DNA Star, BLAST (National Center for Biotechnology Information), BCM (Baylor College of Medicine) Launcher, etc. BLAST can be used to calculate amino acid sequence identity, amino acid sequence homology, and nucleotide sequence identity. These calculations can be made along the entire length of each of the target sequences which are to be compared.

Afler two sequences have been aligned, a “percent sequence identity” can be determined. For these purposes, it is convenient to refer to a Reference Sequence and a Compared Sequence, where the Compared Sequence is compared to the Reference Sequence. Percent sequence identity can be determined according to the following formula: Percent Identity=100 [1−(C/R)], wherein C is the number of differences between the Reference Sequence and the Compared Sequence over the length of alignment between the Reference Sequence and the Compared Sequence where (i) each base or amino acid in the Reference Sequence that does not have a corresponding aligned base or amino acid in the Compared Sequence, (ii) each gap in the Reference Sequence, (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

Percent sequence identity can also be determined by other conventional methods, e.g., as described in Altschul et al., Bull. Math. Bio. 48: 603-616, 1986 and Henikoff and Henikoff, Proc. Natl. Acad. Sci. USA 89:10915-10919, 1992.

Specific Polynucleotide Probes

A polynucleotide of the present invention can comprise any continuous nucleotide sequence of SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, sequences which share sequence identity thereto, or complements thereof. The term “probe” refers to any substance that can be used to detect, identify, isolate, etc., another substance. A polynucleotide probe is comprised of nucleic acid can be used to detect, identify, etc., other nucleic acids, such as DNA and RNA.

These polynucleotides can be of any desired size that is effective to achieve the specificity desired. For example, a probe can be from about 7 or 8 nucleotides to several thousand nucleotides, depending upon its use and purpose. For instance, a probe used as a primer PCR can be shorter than a probe used in an ordered array of polynucleotide probes. Probe sizes vary, and the invention is not limited in any way by their size, e.g., probes can be from about 7-2000 nucleotides, 7-1000, 8-700, 8-600, 8-500, 8-400, 8-300, 8-150, 8-100, 8-75, 7-50, 10-25, 14-16,at least about 8, at least about 10, 11, 12, 13, 14, 15, 16, 17, 18, 19, 20, 21, 22, 23, 24, 25, 26, or more, etc. The polynucleotides can have non-naturally-occurring nucleotides, e.g., inosine, AZT, 3TC, etc. The polynucleotides can have 100% sequence identity or complementarity to a sequence of SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, or it can have mismatches or nucleotide substitutions, e.g., 1, 2, 3, 4, or 5 substitutions. The probes can be single-stranded or double-stranded.

In accordance with the present invention, a polynucleotide can be present in a kit, where the kit includes, e.g., one or more polynucleotides, a desired buffer (e.g., phosphate, tris, etc.), detection compositions, RNA or cDNA from different tissues to be used as controls, libraries, etc. The polynucleotide can be labeled or unlabeled, with radioactive or non-radioactive labels as known in the art. Kits can comprise one or more pairs of polynucleotides for amplifying nucleic acids specific for differentially regulated cancer genes, e.g., comprising a forward and reverse primer effective in PCR. These include both sense and anti-sense orientations. For instance, in PCR-based methods (such as RT-PCR), a pair of primers are typically used, one having a sense sequence and the other having an antisense sequence.

Another aspect of the present invention is a nucleotide sequence that is specific to, or for, a selective polynucleotide. The phrases “specific for” or “specific to” a polynucleotide have a functional meaning that the polynucleotide can be used to identify the presence of one or more target genes in a sample and distinguish them from non-target genes. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, e.g., in the nucleotide sequences of SEQ ID NOS 1, 3, 5, 7, 9, 11, 13, 15, 17, 19,21, 23, 25, 27, 29, 45, 47, 50, 48, 52, 54, 56, 58, 60, 62, 64, 66, 68, 70, 72, 74, 84, 85, 86, 88, 95, and/or 99, and which is characteristic of that target sequence, and substantially no non-target sequences. A probe or mixture of probes can comprise a sequence or sequences that are specific to a plurality of target sequences, e.g., where the sequence is a consensus sequence, a functional domain, etc., e.g., capable of recognizing a family of related genes. Such sequences can be used as probes in any of the methods described herein or incorporated by reference. Both sense and antisense nucleotide sequences are included. A specific polynucleotide according to the present invention can be determined routinely.

A polynucleotide comprising a specific sequence can be used as a hybridization probe to identify the presence of, e.g., human or mouse polynucleotide, in a sample comprising a mixture of polynucleotides, e.g., on a Northern blot. Hybridization can be performed under high stringent conditions (see, above) to select polynucleotides (and their complements which can contain the coding sequence) having at least 90%, 95%, 99%, etc., identity (i.e., complementarity) to the probe, but less stringent conditions can also be used. A specific polynucleotide sequence can also be fused in-flame, at either its 5′ or 3′ end, to various nucleotide sequences as mentioned throughout the patent, including coding sequences for enzymes, detectable markers, GFP, etc, expression control sequences, etc.

A polynucleotide probe, especially one that is specific to a polynucleotide of the present invention, can be used in gene detection and hybridization methods as already described. In one embodiment, a specific polynucleotide probe can be used to detect whether a particular tissue or cell-type is present in a target sample. To carry out such a method, a selective polynucleotide can be chosen which is characteristic of the desired target tissue. Such polynucleotide is preferably chosen so that it is expressed or displayed in the target tissue, but not in other tissues which are present in the sample. For instance, if detection of prostate or breast cancer is desired, it may not matter whether the selective polynucleotide is expressed in other tissues, as long as it is not expressed in cells normally present in blood, e.g., peripheral blood mononuclear cells. Starting from the selective polynucleotide, a specific polynucleotide probe can be designed which hybridizes (if hybridization is the basis of the assay) under the hybridization conditions to the selective polynucleotide, whereby the presence of the selective polynucleotide can be determined.

Probes which are specific for polynucleotides of the present invention can also be prepared using involve transcription-based systems, e.g., incorporating an RNA polymerase promoter into a selective polynucleotide of the present invention, and then transcribing anti-sense RNA using the polynucleotide as a template. See, e.g., U.S. Pat. No. 5,545,522.

Polynucleotide Composition

A polynucleotide according to the present invention can comprise, e.g., DNA, RNA, synthetic polynucleotide, peptide polynucleotide, modified nucleotides, dsDNA, ssDNA, ssRNA, dsRNA, and mixtures thereof. A polynucleotide can be single- or double-stranded, triplex, DNA:RNA, duplexes, comprise hairpins, and other secondary structures, etc. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

Various modifications can be made to the polynucleotides, such as attaching detectable markers (avidin, biotin, radioactive elements, fluorescent tags and dyes, energy transfer labels, energy-emitting labels, binding partners, etc.) or moieties which improve hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967, 5,476,925, and 5,478,893.

Polynucleotide according to the present invention can be labeled according to any desired method. The polynucleotide can be labeled using radioactive tracers such as ³²P, ³⁵S, ³H, or ¹⁴C, to mention some commonly used tracers. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

Nucleic Acid Detection Methods

Another aspect of the present invention relates to methods and processes for detecting differentially regulated cancer genes. Detection methods have a variety of applications, including for diagnostic, prognostic, forensic, and research applications. To accomplish gene detection, a polynucleotide in accordance with the present invention can be used as a “probe.” The term “probe” or “polynucleotide probe” has its customary meaning in the art, e.g., a polynucleotide which is effective to identify (e.g., by hybridization), when used in an appropriate process, the presence of a target polynucleotide to which it is designed. Identification can involve simply determining presence or absence, or it can be quantitative, e.g., in assessing amounts of a gene or gene transcript present in a sample. Probes can be useful in a variety of ways, such as for diagnostic purposes, to identify homologs, and to detect, quantitate, or isolate a polynucleotide of the present invention in a test sample.

Assays can be utilized which permit quantification and/or presencelabsence detection of a target nucleic acid in a sample. Assays can be performed at the single-cell level, or in a sample comprising many cells, where the assay is “averaging” expression over the entire collection of cells and tissue present in the sample. Any suitable assay format can be used, including, but not limited to, e.g., Southern blot analysis, Northern blot analysis, polymerase chain reaction (CTCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115, 1997), ligase chain reaction (“LCR”) (EP 320 308), one-sided PCR (Ohara et al., Proc. Natl. Acad. Sci., 86:5673-5677, 1989), indexing methods (e.g., U.S. Pat. No. 5,508,169), in situ hybridization, differential display (e.g., Liang et al., Nucl. Acid. Res., 21:3269-3275, 1993; U.S. Pat. Nos. 5,262,311, 5,599,672 and 5,965,409; W097/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970, 1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), Qbeta Replicase (PCT/US87/00880), Strand Displacement Amplification (“SDA”), Repair Chain Reaction (“RCR”), nuclease protection assays, subtraction-based methods, Rapid-Scan™, etc. Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280, 1991; U.S. Pat. Nos. 5,210,015 and 5,994,063), real-time fluorescence-based monitoring (e.g., U.S. Pat. 5,928,907), molecular energy transfer labels (e.g., U.S. Pat. Nos. 5,348,853, 5,532,129, 5,565,322, 6,030,787, and 6,117,635; Tyagi and Kramer, Nature Biotech., 14:303-309, 1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat No. 5,723,290). These and other methods can be carried out conventionally, e.g., as described in the mentioned publications.

Many of such methods may require that the polynucleotide is labeled, or comprises a particular nucleotide type useful for detection. The present invention includes such modified polynucleotides that are necessary to carry out such methods. Thus, polynucleotides can be DNA, RNA, DNA:RNA hybrids, PNA, etc., and can comprise any modification or substituent which is effective to achieve detection.

Detection can be desirable for a variety of different purposes, including research, diagnostic, prognostic, and forensic. For diagnostic purposes, it may be desirable to identify the presence or quantity of a polynucleotide sequence in a sample, where the sample is obtained from tissue, cells, body fluids, etc. In a preferred method as described in more detail below, the present invention relates to a method of detecting a polynucleotide comprising, contacting a target polynucleotide in a test sample with a polynucleotide probe under conditions effective to achieve hybridization between the target and probe; and detecting hybridization.

Any test sample in which it is desired to identify a polynucleotide or polypeptide thereof can be used, including, e.g., blood, urine, saliva, stool (for extracting nucleic acid, see, e.g., U.S. Pat. No. 6,177,251), swabs comprising tissue, biopsied tissue, tissue sections, cultured cells, etc.

Polynucleotides can be used in wide range of methods and compositions, including for detecting, diagnosing, staging, grading, assessing, prognosticating, etc. diseases and disorders associated with differentially regulated cancer genes, for monitoring or assessing therapeutic and/or preventative measures, in ordered arrays, etc. Any method of detecting genes and polynucleotides can be used; certainly, the present invention is not to be limited how such methods are implemented.

Along these lines, the present invention relates to methods of detecting differentially regulated cancer genes in a sample comprising nucleic acid. Such methods can comprise one or more the following steps in any effective order, e.g., contacting said sample with a polynucleotide probe under conditions effective for said probe to hybridize specifically to nucleic acid in said sample, and detecting the presence or absence of probe hybridized to nucleic acid in said sample, wherein said probe is a polynucleotide which is selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, a polynucleotide having, e.g., about 70%, 80%, 85%, 90%, 95%, 99%, or more sequence identity thereto, effective or specific framents thereof, or complements thereto. The detection method can be applied to any sample, e.g., cultured primary, secondary, or established cell lines, tissue biopsy, blood, urine, stool, cerebral spinal fluid, and other bodily fluids, for any purpose.

Contacting the sample with probe can be carried out by any effective means in any effective environment It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.). When hybridization is the chosen means of achieving detection, the probe and sample can be combined such that the resulting conditions are functional for said probe to hybridize specifically to nucleic acid in said sample.

The phrase “hybridize specifically” indicates that the hybridization between single-stranded polynucleotides is based on nucleotide sequence complementarity. The effective conditions are selected such that the probe hybridizes to a preselected and/or definite target nucleic acid in the sample. For instance, if detection of a polynucleotide set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 is desired, a probe can be selected which can hybridize to such target gene under high stringent conditions, without significant hybridization to other genes in the sample. To detect homologs of a polynucleotide set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, the effective hybridization conditions can be less stringent, and/or the probe can comprise codon degeneracy, such that a homolog is detected in the sample.

As already mentioned, the methods can be carried out by any effective process, e.g., by Northern blot analysis, polymerase chain reaction (PCR), reverse transcriptase PCR, RACE PCR, in situ hybridization, etc., as indicated above. When PCR based techniques are used, two or more probes are generally used. One probe can be specific for a defined sequence which is characteristic of a selective polynucleotide, but the other probe can be specific for the selective polynucleotide, or specific for a more general sequence, e.g., a sequence such as polyA which is characteristic of mRNA, a sequence which is specific for a promoter, ribosome binding site, or other transcriptional features, a consensus sequence (e.g., representing a functional domain). For the former aspects, 5′ and 3′ probes (e.g., polyA, Kozak, etc.) are preferred which are capable of specifically hybridizing to the ends of transcripts. When PCR is utilized, the probes can also be referred to as “primers” in that they can prime a DNA polymerase reaction.

In addition to testing for the presence or absence of polynucleotides, the present invention also relates to determining the amounts at which polynucleotides of the present invention are expressed in sample and determining the differential expression of such polynucleotides in samples. Such methods can involve substantially the same steps as described above for presence/absence detection, e.g., contacting with probe, hybridizing, and detecting hybridized probe, but using more quantitative methods and/or comparisons to standards.

The amount of hybridization between the probe and target can be determined by any suitable methods, e.g., PCR, RT-PCR, RACE PCR, Northern blot, polynucleotide microarrays, Rapid-Scan, etc., and includes both quantitative and qualitative measurements. For further details, see the hybridization methods described above and below. Determining by such hybridization whether the target is differentially expressed (e.g., up-regulated or down-regulated) in the sample can also be accomplished by any effective means. For instance, the target's expression pattern in the sample can be compared to its pattern in a known standard, such as in a normal tissue, or it can be compared to another gene in the same sample. When a second sample is utilized for the comparison, it can be a sample of normal tissue that is known not to contain diseased cells. The comparison can be performed on samples which contain the same amount of RNA (such as polyadenylated RNA or total RNA), or, on RNA extracted from the same amounts of starting tissue. Such a second sample can also be referred to as a control or standard. Hybridization can also be compared to a second target in the same tissue sample. Experiments can be performed that determine a ratio between the target nucleic acid and a second nucleic acid (a standard or control), e.g., in a normal tissue. When the ratio between the target and control are substantially the same in a normal and sample, the sample is determined or diagnosed not to contain cells. However, if the ratio is different between the normal and sample tissues, the sample is determined to contain cancer cells. The approaches can be combined, and one or more second samples, or second targets can be used. Any second target nucleic acid can be used as a comparison, including “housekeeping” genes, such as beta-actin, alcohol dehydrogenase, or any other gene whose expression does not vary depending upon the disease status of the cell.

Methods of Identifying Polymorphisms, Mutations, etc., of Differentially Regulated Cancer Genes

Polynucleotides of the present invention can also be utilized to identify mutant alleles, SNPs, gene rearrangements and modifications, and other polymorphisms of the wild-type gene. Mutant alleles, polymorphisms, SNPs, etc., can be identified and isolated from subjects with diseases that are known, or suspected to have, a genetic component. Identification of such genes can be carried out routinely (see, above for more guidance), e.g., using PCR, hybridization techniques, direct sequencing, mismatch reactions (see, e.g., above), RFLP analysis, SSCP (e.g., Orita et al., Proc. Natl. Acad. Sci., 86:2766, 1992), etc., where a polynucleotide having a sequence selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 is used as a probe. The selected mutant alleles, SNPs, polymorphisms, etc., can be used diagnostically to determine whether a subject has, or is susceptible to a disorder associated with differentially regulated cancer genes, as well as to design therapies and predict the outcome of the disorder. Methods involve, e.g., diagnosing a disorder associated with differentially regulated cancer genes or determining susceptibility to a disorder, comprising, detecting the presence of a mutation in a gene represented by a polynucleotide selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99. The detecting can be carried out by any effective method, e.g., obtaining cells from a subject, determining the gene sequence or structure of a target gene (using, e.g., mRNA, cDNA, genomic DNA, etc), comparing the sequence or structure of the target gene to the structure of the normal gene, whereby a difference in sequence or structure indicates a mutation in the gene in the subject. Polynucleotides can also be used to test for mutations, SNPs, polymorphisms, etc., e.g., using mismatch DNA repair technology as described in U.S. Pat. No. 5,683,877; U.S. Pat. No. 5,656,430; Wu et al., Proc. Natl. Acad. Sci., 89:8779-8783, 1992.

The present invention also relates to methods of detecting polymorphisms in differentially regulated cancer genes, comprising, e.g., comparing the structure of: genomic DNA comprising all or part of a differentially regulated cancer gene, mRNA comprising all or part of a differentially regulated cancer gene, cDNA comprising all or part of a differentially regulated cancer gene, or a polypeptide comprising all or part of differentially regulated cancer gene, with the structure of a differentially regulated cancer gene,e.g., as set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99. The methods can be carried out on a sample from any source, e.g., cells, tissues, body fluids, blood, urine, stool, hair, egg, sperm, cerebral spinal fluid, etc.

These methods can be implemented in many different ways. For example, “comparing the structure” steps include, but are not limited to, comparing restriction maps, nucleotide sequences, amino acid sequences, RFLPs, DNAase sites, DNA methylation fingerprints (e.g., U.S. Pat. No. 6,214,556), protein cleavage sites, molecular weights, electrophoretic mobilities, charges, ion mobility, etc., between a standard differentially regulated cancer genes and a test differentially regulated cancer genes. The term “structure” can refer to any physical characteristics or configurations which can be used to distinguish between nucleic acids and polypeptides. The methods and instruments used to accomplish the comparing step depends upon the physical characteristics which are to be compared. Thus, various techniques are contemplated, including, e.g., sequencing machines (both amino acid and polynucleotide), electrophoresis, mass spectrometer (U.S. Pat. Nos. 6,093,541, 6,002,127), liquid chromatography, HPLC, etc.

To carry out such methods, “all or part” of the gene or polypeptide can be compared. For example, if nucleotide sequencing is utilized, the entire gene can be sequenced, including promoter, introns, and exons, or only parts of it can be sequenced and compared, e.g., exon 1, exon 2, etc.

Mutagenesis

Mutated polynucleotide sequences of the present invention are useful for various purposes, e.g., to create mutations of the polypeptides they encode, to identify functional regions of genomic DNA, to produce probes for screening libraries, etc. Mutagenesis can be carried out routinely according to any effective method, e.g., oligonucleotide-directed (Smith, M., Ann. Rev. Genet. 19:423-463, 1985), degenerate oligonucleotiddirected (Hill et al., Method Enzymology, 155:558-568, 1987), region-specific (Myers et al., Science, 229:242-246, 1985; Derbyshire et al., Gene, 46:145, 1986; Ner et al., DNA, 7:127, 1988), inker-scanning (McKnight and Kingsbury, Science, 217:316-324, 1982), directed using PCR, recursive ensemble mutagenesis (Arkin and Yourvan, Proc. Natl. Acad. Sci., 89:7811-7815, 1992), random mutagenesis (e.g., U.S. Pat. Nos. 5,096,815; 5,198,346; and 5,223,409), site-directed mutagenesis (e.g., Walder et al., Gene, 42:133, 1986; Bauer et al., Gene, 37:73, 1985; Craik, Bio Techniques, January 1985, 12-19; Smith et al., Genetic Engineering: Principles and Methods, Plenum Press, 1981), phage display (e.g., Lowman et al., Biochem. 30:10832-10837, 1991; Ladner et al., U.S. Pat. No. 5,223,409; Huse, WIPO Publication WO 92/06204), etc. Desired sequences can also be produced by the assembly of target sequences using mutually priming oligonucleotides (Uhllmann, Gene, 71:2940, 1988). For directed mutagenesis methods, analysis of the three-dimensional structure of the differentially regulated cancer genes polypeptide can be used to guide and facilitate making mutants which effect polypeptide activity. Sites of substrate-enzyme interaction or other biological activities can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904, 1992; Wlodaver et al., FEBS Lett. 309:59-64, 1992.

In addition, libraries of differentially regulated cancer genes and fragments thereof can be used for screening and selection of differentially regulated cancer genes variants. For instance, a library of coding sequences can be generated by treating a double-stranded DNA with a nuclease under conditions where the nicking occurs, e.g., only once per molecule, denaturing the double-stranded DNA, renaturing it to for double-stranded DNA that can include sense/antisense pairs from different nicked products, removing single-stranded portions from reformed duplexes by treatment with S1 nuclease, and ligating the resulting DNAs into an expression vector. By this method, expression libraries can be made comprising “mutagenized” differentially regulated cancer genes. The entire coding sequence or parts thereof can be used.

Polynucleotide Expression, Polypeptides Produced thereby, and Specific-Binding Partners Thereto.

A polynucleotide according to the present invention can be expressed in a variety of different systems, in vitro and in vivo, according to the desired purpose. For example, a polynucleotide can be inserted into an expression vector, introduced into a desired host, and cultured under conditions effective to achieve expression of a polypeptide coded for by the polynucleotide, to search for specific binding partners. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding polynucleotide is adjacent to a dhfr gene), cycloheximide, cell densities, culture dishes, etc. A polynucleotide can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a polynucleotide of the present invention has been introduced is a transformed host cell. The polynucleotide can be extrachromosomal or integrated into a chromosome(s) of the host cell. It can be stable or transient. An expression vector is selected for its compatibility with the host cell. Host cells include, mammalian cells, e.g., COS, CV1, BHK, CHO, Hela, LIX, NIH 3T3,

PC-3 (CRL-1435), LNCaP (CRL-1740), CA-HPV-10 (CRL-2220), PZ-HPV-7 (CRL-2221), MDA-PCa 2b (CRL-2422), 22Rv1 (CRL2505), NCI-H660 (CRL-5813), HS 804.Sk (CRL-7535), LNCaP-FGF (CRL-10995), RWPE-1 (CRL,11609), RWPE-2 (CRI11610), PWR-1E (CRL 11611), rat MAT-Ly-LuB-2 (CRL-2376), and other primary and established prostate and prostate cancer cell lines, ZR-75-1 (ATCC CRL-1500), ZR-75-30 (ATCC CRL-1504), UACC-812 (ATCC CRL,1897), UACC-893 (ATCC CRL-1902), HCC38 (ATCC CRL-2314), HCC70 (CRL-2315), and other HCC cell lines (e.g., as deposited with the ATCC), AU565 (ATCC CRL-2351), Hs 496.T (ATCC CRL-7303), Hs 748.T (ATCC CRL-7486), SW527 (ATCC CRL-7940), 184A1 (ATCC CRL-8798), MCF cell lines (e.g., 10A and others deposited with the ATCC), MDA-MB-134-VI (ATCC HTB-23 and other MDA cell lines), SK-BR-3 (ATCC HTB-30), ME-180 (ATCC HTB-33), Hs 578Bst (ATCC HTB-125), Hs 578T (ATCC HTB-126), T-47D (ATCC HTB-133), and other primary and established breast and breast cancer cell lines, insect cells, such as 59 (S. frugipeda) and Drosophila, bacteria, such as E. coli, Streptococcus, bacillus, yeast, such as Sacharomyces, S. cerevisiae, fungal cells, plant cells, embryonic or adult stem cells (e.g., mammalian, such as mouse or human).

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type'specific elements, or sequences which allow selective or specific cell expression. Promoters that can be used to drive its expression, include, e.g., the endogenous promoter, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Polynucleotide Res., 12(18):7035-7056, 1984; Dunn and Studier. J. Mol. Bio., 166:477-435, 1984; U.S. Pat. No. 5,891,636; Studier et al., Gene Epression Technology, Methods in Enzymology, 85:60-89, 1987. In addition, as discussed above, translational signals (including in-frame insertions) can be included.

When a polynucleotide is expressed as a heterologous gene in a transfected cell line, the gene is introduced into a cell as described above, under effective conditions in which the gene is expressed. The term “heterologous” means that the gene has been introduced into the cell line by the “hand-of-man.” Introduction of a gene into a cell line is discussed above. The transfected (or transformed) cell expressing the gene can be lysed or the cell line can be used intact.

For expression and other purposes, a polynucleotide can contain codons found in a naturally-occurring gene, transcript, or cDNA, for example, e.g., as set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, or it can contain degenerate codons coding for the same amino acid sequences. For instance, it may be desirable to change the codors in the sequence to optimize the sequence for expression in a desired host. See, e.g., U.S. Pat. Nos. 5,567,600 and 5,567,862.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. Another approach is express the polypeptide recombinantly with an affinity tag (Flag epitope, HA epitope, myc epitope, 6× His, maltose binding protein, chitinase, etc) and then purify by anti-tag antibody-conjugated affinity chromatography.

The present invention also relates to polypeptides of differentially regulated cancer genes, e.g., an isolated human differentially regulated cancer gene polypeptide comprising or having the amino acid sequence set forth in SEQ D) NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99, an isolated mammalian differentially regulated cancer genes polypeptide comprising an amino acid sequence, e.g., having at least 90%, 95%, 99%, or more amino acid sequence identity to the amino acid sequence set forth in SEQ ID NOS 2, 4, 6, 8, 10, 12, 14, 16, 18, 20, 22, 24, 26, 28, 30, 46, 51, 49, 53, 55, 57, 59, 61, 63, 65, 67, 69, 71, 73, 75, 87, 89, 96 and/or 100, and optionally having one or more of differentially regulated cancer genes activities. Fragments specific to differentially regulated cancer genes can also used, e.g., to produce antibodies or other immune responses, as competitors, etc. These fragments can be referred to as-being “specific for” a differentially regulated cancer gene. The latter phrase, as already defined, indicates that the peptides are characteristic of a particular genes, and that the defined sequences are substantially absent from all other protein types. Such polypeptides can be of any size which is necessary to confer specificity, e.g., 5, 8, 10, 12, 15, 20, etc.

The present invention also relates to specific-binding partners. These include antibodies which are specific for polypeptides encoded by polynucleotides of the present invention, as well as other binding-partners which interact with polynucleotides and polypeptides of the present invention. Protein-protein interactions between differentially regulated cancer genes and other polypeptides and binding partners can be identified using any suitable methods, e.g., protein binding assays (e.g., filtration assays, chromatography, etc.), yeast two-hybrid system (Fields and Song, Nature, 340: 245-247, 1989), protein arrays, gel-shift assays, FRET (fluorescence resonance energy transfer) assays, etc. Nucleic acid interactions (e.g., protein-DNA or protein-RNA) can be assessed using gel-shift assays, e.g., as carried out in U.S. Pat. Nos. 6,333,407 and 5,789,538.

Antibodies, e.g., polyclonal, monoclonal, recombinant, chimeric, humanized, single-chain, Fab, and fragments thereof, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. The antibodies can be IgM, IgG, subtypes, IgG2a, IgG1, etc. Antibodies, and immune responses, can also be generated by administering naked DNA See, e.g., U.S. Pat Nos. 5,703,055; 5,589,466; 5,580,859. Antibodies can be used from any source, including, goat, rabbit, mouse, chicken (e.g., IgY; see, Duan, W0/029444 for methods of making antibodies in avian hosts, and harvesting the antibodies from the eggs). An antibody specific for a polypeptide means that the antibody recognizes a defined sequence of amino acids within or including the polypeptide. Other specific binding partners include, e.g., aptamers and PNA. Antibodies can be prepared against specific epitopes or domains of a differentially regulated cancer gene. Antibodies can also be humanized, e.g., where they are to be used therapeutically.

The term “antibody” as used herein includes intact molecules as well as fragments thereof, such as Fab, F(ab′)2, and Fv which are capable of binding to an epitopic determinant present in Bin1 polypeptide. Such antibody fragments retain some ability to selectively bind with its antigen or receptor. The term “epitope” refers to an antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. Antibodies can be prepared against specific epitopes or polypeptide domains.

Antibodies which bind to differentially regulated cancer genes polypeptides of the present invention can be prepared using an intact polypeptide or fragments containing small peptides of interest as the immunizing antigen. For example, it may be desirable to produce antibodies that specifically bind to the N- or C-terminal domains of differentially regulated cancer genes. The polypeptide or peptide used to immunize an animal which is derived from translated cDNA or chemically synthesized which can be conjugated to a carrier protein, if desired. Such commonly used carriers which are chemically coupled to the immunizing peptide include keyhole limpet hemocyanin (KLH), thyroglobulin, bovine serum albumin (BSA), and tetanus toxoid.

Methods of Detecting Polypeptides

Polypeptides coded for by differentially regulated cancer genes of the present invention can be detected, visualized, determined, quantitated, etc. according to any effective method, useful methods include, e.g., but are not limited to, immunoassays, RIA (radioimmunassay), ELISA, (enzyme-linked-immunosorbent assay), inumunoflourescence, flow, cytometry, histology, electron microscopy, light microscopy, in situ assays, immunoprecipitation, Western blot, etc.

Inmunoassays may be carried in liquid or on biological support. For instance, a sample (e.g., blood, stool, urine, cells, tissue,cerebral spinal fluid, body fluids, etc.) can be brought in contact with and immobilized onto a solid phase support or carrier such as nitrocellulose, or other solid support that is capable of immobilizing cells, cell particles or soluble proteins. The support may then be washed with suitable buffers followed by treatment with the detectably labeled differentially regulated cancer genes specific antibody. The solid phase support can then be washed with a buffer a second time to remove unbound antibody. The amount of bound label on solid support may then be detected by conventional means.

A “solid phase support or carrier” includes any support capable of binding an antigen, antibody, or other specific binding partner. Supports or carriers include glass, polystyrene, polypropylene, polyethylene, dextran, nylon, amylases, natural and modified celluloses, polyacrylamides, and magnetite. A support material can have any structural or physical configuration Thus, the support configuration may be spherical, as in a bead, or cylindrical, as in the inside surface of a test tube, or the external surface of a rod. Alternatively, the surface may-be flat such as a sheet, test strip, etc.

One of the many ways in which gene peptide-specific antibody can be detectably labeled is by linking it to an enzyme and using it in an enzyme immunoassay (EIA). See, e.g., Voller, A., “The Enzyme Linked Immunosorbent Assay (ELISA),” 1978, Diagnostic Horizons 2, 1-7, Microbiological Associates Quarterly Publication, Walkersville, Md.); Voller, A. et al., 1978, J. Clin. Pathol. 31, 507-520; Butler, J. E., 1981, Meth. Enzymol. 73, 482-523; Maggio, E. (ed.), 1980, Enzyme Immunoassay, CRC Press, Boca Raton, Fla. The enzyme which is bound to the antibody will react with an appropriate substrate, preferably a chromogenic substrate, in such a manner as to produce a chemical moiety that can be detected, for example, by spectrophotometric, fluorimetric or by visual means. Enzymes that can be used to detectably label the antibody include, but are not limited to, malate dehydrogenase, staphylococcal nuclease, delta-5-steroid isomerase, yeast alcohol dehydrogenase, .alpha.-glycerophosphate, dehydrogenase, triose phosphate isomerase, horseradish peroxidase, alkaline phosphatase, asparaginase, glucose oxidase, beta.-galactosidase, ribonuclease, urease, catalase, glucose-6-phosphate dehydrogenase, glucoamylase and acetylcholinesterase. The detection can be accomplished by colorimetric methods that employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect differentially regulated cancer genes peptides through the use of a radioimmunoassay (RIA). See, e.g., Weintraub, B., Principles of Radioimmunoassays, Seventh Training Course on Radioligand Assay Techniques, The Endocrine Society, March, 1986. The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoein, phycocyanin, allophycocyanin, o-phthaldehyde and fluorescamme. The antibody can also be detectably labeled using fluorescence emitting metals such as those in the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Diagnostic

The present invention also relates to methods and compositions for diagnosing prostate or breast cancer, or determining susceptibility to said cancer, using polynucleotides, polypeptides, and specific-binding partners of the present invention to detect, assess, determine, etc., the expression of differentially regulated cancer genes and their polypeptide products. In such methods, the gene can serve as a marker for the disorder, e.g., where the gene, when mutant, is a direct cause of the disorder; where the gene is affected by another gene(s) which is directly responsible for the disorder, e.g., when the gene is part of the same signaling pathway as the directly responsible gene; and, where the gene is chromosomally linked to the gene(s) directly responsible for the disorder, and segregates with it. Many other situations are possible. To detect, assess, determine, etc., a probe specific for the gene can be employed as described above and below. Any method of detecting and/or assessing the gene can be used, including detecting expression of the gene using polynucleotides, antibodies, or other specific-binding partners.

The present invention relates to methods of diagnosing a cancer associated with a differentially regulated cancer gene of the present invention, or determining a subject's susceptibility to such cancer, comprising, e.g., assessing the expression of a gene of the present invention in a tissue sample comprising tissue or cells suspected of having cancer. The phrase “diagnosing” indicates that it is determined whether the sample has the disorder. A “disorder” means, e.g., any abnormal condition as in a disease or malady. “Determining a subject's susceptibility to a disease or disorder” indicates that the subject is assessed for whether s/he is predisposed to get such a disease or disorder, where the predisposition is indicated by abnormal expression of the gene (e.g., gene mutation, gene expression pattern is not normal, etc.). Predisposition or susceptibility to a disease may result when a such disease is influenced by epigenetic, environmental, etc., factors. Diagnosing includes prenatal screening where samples from the fetus or embryo (e.g., via amniocentesis or CV sampling) are analyzed for the expression of the gene.

By the phrase “assessing expression of a differentially regulated gene,” it is meant that the functional status of the gene is evaluated. This includes, but is not limited to, measuring expression levels of said gene, determining the genomic structure of said gene, determining the mRNA structure of transcripts from said gene, or measuring the expression levels of polypeptide coded for by said gene. Thus, the term “assessing expression” includes evaluating the all aspects of the transcriptional and translational machinery of the gene. For instance, if a promoter defect causes, or is suspected of causing, the disorder, then a sample can be evaluated (i.e., “assessed”) by looking (e.g., sequencing or restriction mapping) at the promoter sequence in the gene, by detecting transcription products (e.g., RNA), by detecting translation product (e.g., polypeptide). Any measure of whether the gene is functional can be used, including, polypeptide, polynucleotide, and functional assays for the gene's biological activity.

In making the assessment, it can be useful to compare the results to a normal gene, e.g., a gene which is not associated with the disorder. The nature of the comparison can be determined routinely, depending upon how the assessing is accomplished. If, for example, the mRNA levels of a sample is detected, then the mRNA levels of a normal can serve as a comparison, or a gene which is known not to be affected by the disorder. Methods of detecting mRNA are well known, and discussed above, e.g., but not limited to, Northern blot analysis, polymerase chain reaction (PCR), reverse transcriptase PCR, RACE PCR, etc. Similarly, if polypeptide production is used to evaluate the gene, then the polypeptide in a normal tissue sample can be used as a comparison, or, polypeptide from a different gene whose expression is known not to be affected by the disorder. These are only examples of how such a method could be carried out.

The genes and polypeptides of the present invention can be used to identify, detect, stage, determine the presence of, prognosticate, treat, study, etc., breast, prostate, and other cancer. The present invention relates to methods of identifying a genetic basis for a disease or disease-susceptibility, comprising, e.g., determining the association of a cancer or cancer susceptibility with a gene of the present invention. An association between a disease or disease-susceptibility and nucleotide sequence includes, e.g., establishing (or finding) a correlation (or relationship) between a DNA marker (e.g., gene, VNTR, polymorphism, EST, etc.) and a particular disease state. Once a relationship is identified, the DNA marker can be utilized in diagnostic tests and as a drug target. Any region of the gene can be used as a source of the DNA marker, exons, introns, intergenic regions, etc.

Human linkage maps can be constructed to establish a relationship between a gene and cancer. Typically, polymorphic molecular markers (e.g., STRP's, SNP's, RFLP's, VNmR's) are identified within the region, linkage and map distance between the markers is then established, and then linkage is established between phenotype and the various individual molecular markers. Maps can be produced for an individual family, selected populations, patient populations, etc. In general, these methods involve identifying a marker associated with the disease (e.g., identifying a polymorphism in a family which is linked to the disease) and then analyzing the surrounding DNA to identity the gene responsible for the phenotype. See, e.g., Kruglyak et al., Am. J. Hum. Genet., 58, 1347-1363, 1996; Matise et al., Nat. Genet., 6(4):384-90, 1994.

Assessing the effects of therapeutic and preventative interventions (e.g., administration of a drug, chemotherapy, radiation, etc.) on cancer is a major effort in drug discovery, clinical medicine, and pharmacogenomics. The evaluation of therapeutic and preventative measures, whether experimental or already in clinical use, has broad applicability, e.g., in clinical trials, for monitoring the status of a patient, for analyzing and assessing animal models, and in any scenario involving disease treatment and prevention. Analyzing the expression profiles of polynucleotides of the present invention can be utilized as a parameter by which interventions are judged and measured. Treatment of a disorder can change the expression profile in some manner which is prognostic or indicative of the drug's effect on it. Changes in the profile can indicate, e.g., drug toxicity, return to a normal level, etc. Accordingly, the present invention also relates to methods of monitoring or assessing a therapeutic or preventative measure (e.g., chemotherapy, radiation, anti-neoplastic drugs, antibodies, etc.) in a subject having a cancer, or, susceptible to cancer, comprising, e.g., detecting the expression levels of differentially regulated cancer genes. A subject can be a cell-based assay system, non-human animal model, human patient, etc. Detecting can be accomplished as described for the methods above and below. By “therapeutic or preventative intervention,” it is meant, e.g., a drug administered to a patient, surgery, radiation, chemotherapy, and other measures taken to prevent, treat, or diagnose a disorder.

The present invention also relates to methods of using differentially regulated cancer genes binding partners, such as antibodies, to deliver active agents to the cancer for a variety of different purposes, including, e.g., for diagnostic, therapeutic (e.g., to treat cancer), and research purposes. Methods can involve delivering or administering an active agent to the cancer, comprising, e.g., administering to a subject in need thereof, an effective amount of an active agent coupled to a binding partner specific for human differentially regulated cancer genes polypeptide, wherein said binding partner is effective to deliver said active agent specifically to said cancer.

Any type of active agent can be used in combination with a binding partner, including, therapeutic, cytotoxic, cytostatic, chemotherapeutic, anti-neoplastic, anti-proliferative, anti-biotic, etc., agents. A chemotherapeutic agent can be, e.g., DNA-interactive agent, alkylating agent, antimetabolite, tubulin-interactive agent, hormonal agent, hydroxyurea, Cisplatin, Cyclophosphamide, Altretamine, Bleomycin, Dactinomycin, Doxorubicin, Etoposide, Teniposide, paclitaxel, cytoxan, 2-methoxy-carbonyl-amino-benzimidazole, Plicamycin, Methotrexate, Fluorouracil, Fluorodeoxyrridin, CB3717, Azacitidine, Floxuridine, Mercapyopurine, 6-Thioguanine, Pentostatin, Cytarabine, Fludarabine, etc. Agents can also be contrast agents useful in imaging technology, e.g., X-ray, CT, CAT, MRI ultrasound, PET, SPECT, and scintographic.

An active agent can be associated in any manner with a differentially regulated cancer genes binding partner which is effective to achieve its delivery specifically to the target. Specific delivery or targeting indicates that the agent is provided to the cancer, without being substantially provided to other tissues. This is useful especially where an agent is toxic, and specific targeting to the cancer enables the majority of the toxicity to be aimed it, with as small as possible effect on other tissues in the body. The association of the active agent and the binding partner (“coupling”) can be direct, e.g., through chemical bonds between the binding partner and the agent, or, via a linidng agent, or the association can be less direct, e.g., where the active agent is in a liposome, or other carrier, and the binding partner is associated with the liposome surface. In such case, the binding partner can be oriented in such a way that it is able to bind to differentially regulated cancer gene product, e.g., on the cell surface. Methods for delivery of DNA via a cell-surface receptor is described, e.g., in U.S. Pat No. 6,339,139.

Identifying Agent Methods

The present invention also relates to methods of identifying agents, and the agents themselves, which modulate a differentially regulated cancer gene. These agents can be used to modulate the biological activity of the polypeptide encoded for the gene, or the gene, itself. Agents which regulate the gene or its product are useful in variety of different environments, including as medicinal agents to treat or prevent disorders associated with differentially regulated cancer genes and as research reagents to modify the fumction of tissues and cell.

Methods of identifying agents generally comprise steps in which an agent is placed in contact with the gene, its transcription product, its translation product, or other target, and then a determination is performed to assess whether the agent “modulates” the target. The specific method utilized will depend upon a number of factors, including, e.g., the target (i.e., is it the gene or polypeptide encoded by it), the environment (e.g., in vitro or in vivo), the composition of the agent, etc.

For modulating the expression of a differentially regulated cancer gene, a method can comprise, in any effective order, one or more of the following steps, e.g., contacting a differentially regulated cancer gene (e.g., in a cell population) with a test agent under conditions effective for said test agent to modulate the expression of said differentially regulated cancer gene, and determining whether said test agent modulates said differentially regulated cancer gene. An agent can modulate expression of differentially regulated cancer gene at any level, including transcription (e.g., by modulating the promoter), translation, and/or perdurance of the nucleic acid (e.g., degradation, stability, etc.) in the cell.

For modulating the biological activity of differentially regulated cancer gene product, such as a polypeptides, a method can comprise, in any effective order, one or more of the following steps, e.g., contacting a differentially regulated cancer gene polypeptide (e.g., in a cell, lysate, or isolated) with a test agent under conditions effective for said test agent to modulate the biological activity of said polypeptide, and determining whether said test agent modulates said biological activity.

Contacting a differentially regulated cancer gene with the test agent can be accomplished by any suitable method and/or means that places the agent in a position to functionally control expression or biological activity of the gene present in the sample. Functional control indicates that the agent can exert its physiological effect on differentially regulated cancer genes through whatever mechanism it works. The choice of the method and/or means can depend upon the nature of the agent and the condition and type of environment in which the differentially regulated cancer genes is presented, e.g., lysate, isolated, or in a cell population (such as, in vivo, in vitro, organ explants, etc.). For instance, if the cell population is an in vitro cell culture, the agent can be contacted with the cells by adding it directly into the culture medium. If the agent cannot dissolve readily in an aqueous medium, it can be incorporated into liposomes, or another lipophilic carrier, and then administered to the cell culture. Contact can also be facilitated by incorporation of agent with carriers and delivery molecules and complexes, by injection, by infusion, etc.

Agents can be directed to, or targeted to, any part of the polypeptide which is effective for modulating it. For example, agents, such as antibodies and small molecules, can be targeted to cell-surface, exposed, extracellular, ligand binding, functional, etc., domains of the polypeptide. Agents can also be directed to intracellular regions and domains, e.g., regions where the polypeptide couples or interacts with intracellular or intramembrane binding partners.

After the agent has been administered in such a way that it can gain access to differentially regulated cancer genes, it can be determined whether the test agent modulates differentially regulated cancer genes expression or biological activity. Modulation can be of any type, quality, or quantity, e.g., increase, facilitate, enhance, up-regulate, stimulate, activate, amplify, augment, induce, decrease, down-regulate, diminish, lessen, reduce, etc. The modulatory quantity can also encompass any value, e.g., 1%, 5%, 10%, 50%, 75%, 1-fold, 2-fold, 5-fold, 1 0-fold, 100-fold, etc. To modulate differentially regulated cancer genes expression means, e.g., that the test agent has an effect on its expression, e.g., to effect the amount of transcription, to effect RNA splicing, to effect translation of the RNA into polypeptide, to effect RNA or polypeptide stability, to effect polyadenylation or other processing of the RNA, to effect post-transcriptional or post-translational processing, etc. To modulate biological activity means, e.g., that a functional activity of the polypeptide is changed in comparison to its normal activity in the absence of the agent This effect includes, increase, decrease, block inhibit, enhance, etc A test agent can be of any molecular composition, e.g., chemical compounds, biomolecules, such as polypeptides, lipids, nucleic acids, carbohydrates, antibodies, ribozymes, double-stranded RNA, aptamers, etc. For example, if a polypeptide to be modulated is a cell-surface molecule, a test agent can be an antibody that specifically recognizes it and, e.g., causes the polypeptide to be internalized, leading-to its down regulation on the surface of the cell. Such an effect does not have to be permanent, but can require the presence of the antibody to continue the down-regulatory effect. Antibodies can also be used to modulate the biological activity of a polypeptide in a lysate or other cell-free form.

Therapeutics

Selective polynucleotides, polypeptides, and specific-binding partners thereto, can be utilized in therapeutic applications, especially to treat prostate and breast cancers. Useful methods include, but are not limited to, immunotherapy (e.g., using specific-binding partners to polypeptides), vaccination (e.g., using a selective polypeptide or a naked DNA encoding such polypeptide), protein or polypeptide replacement therapy, gene therapy (e.g., germ-line correction, antisense), etc.

Various immunotherapeutic approaches can be used. For instance, unlabeled antibody that specifically recognizes a tissue-specific antigen can be used to stimulate the body to destroy or attack a cancer or other diseased tissue, to cause down-regulation, to produce complement-mediated lysis, to inhibit cell growth, etc., of target cells which display the antigen, e.g., analogously to how c-erbB-2 antibodies are used to treat breast cancer. In addition, antibody can be labeled or conjugated to enhance its deleterious effect, e.g., with radionuclides and other energy emitting entitities, toxins, such as ricin, exotoxin A (ETA), and diphtheria, cytotoxic or cytostatic agents, immunomodulators, chemotherapeutic agents, etc. See, e.g., U.S. Pat. No. 6,107,090.

An antibody or other specific-binding partner can be conjugated to a second molecule, such as a cytotoxic agent, and used for targeting the second molecule to a tissue-antigen positive cell (Vitetta, E. S. et al., 1993, Immunotoxin therapy, in DeVita, Jr., V. T. et al., eds, Cancer: Principles and Practice of Oncology, 4th ed., J. B. Lippincott Co., Philadelphia, 2624-2636). Examples of cytotoxic agents include, but are not limited to, antimetabolites, alkylating agents, anthracyclines, antibiotics, anti-mitotic agents, radioisotopes and chemotherapeutic agents. Further examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, 1-dehydrotestosterone, diptheria toxin, Pseudomonas exotoxin (PE) A, PE40, abrin, elongation factor-2 and glucocorticoid. Techniques for conjugating therapeutic agents to antibodies are well.

In addition to immunotherapy, polynucleotides and polypeptides can be used as targets for non-immunotherapeutic applications, e.g., using compounds which interfere with function, expression (e.g., antisense as a therapeutic agent), assembly, etc. RNA interference can be used in vitro and in vivo to silence differentially regulated cancer genes when its expression contributes to a disease (but also for other purposes, e.g., to identify the gene's function to change a developmental pathway of a cell, etc.). See, e.g., Sharp and Zamore, Science, 287:2431-2433, 2001; Grishok et al., Science, 287:2494, 2001.

Delivery of therapeutic agents can be achieved according to any effective method, including, liposomes, viruses, plasmid vectors, bacterial delivery systems, orally, systemically, etc. Therapeutic agents of the present invention can be admnistered in any form by any effective route, including, e.g., oral, parenteral, enteral, intraperitoneal, topical, transdermal (e.g., using any standard patch), intravenously, ophthalmic, nasally, local, non-oral, such as aerosal, inhalation, subcutaneous, intramuscular, buccal, sublingual, rectal, vaginal, intra-arterial, and intrathecal, etc. They can be administered alone, or in combination with any ingredient(s), active or inactive.

In addition to therapeutics, per se, the present invention also relates to methods of treating a cancer showing altered expression of differentially regulated cancer genes, comprising, e.g., administering to a subject in need thereof a therapeutic agent which is effective for regulating expression of said differentially regulated cancer genes and/or which is effective in treating said disease. The term treating” is used conventionally, e.g., the management or care of a subject for the purpose of combating, alleviating, reducing, relieving, improving the cancer. By the phrase “altered expression,” it is meant that the disease is associated with a mutation in the gene, or any modification to the gene (or corresponding product) which affects its normal function. Thus, expression of a differentially regulated cancer gene refers to, e.g., transcription, translation, splicing, stability of the mRNA or protein product, activity of the gene product, differential expression, etc.

Any agent which “treats” the disease can be used. Such an agent can be one which regulates the expression of the differentially regulated cancer genes. Expression refers to the same acts already mentioned, e.g. transcription, translation, splicing, stability of the mRNA or protein product, activity of the gene product, differential expression, etc. For instance, if the condition was a result of a complete deficiency of the gene product, administration of gene product to a patient would be said to treat the disease and regulate the gene's expression. Many other possible situations are possible, e.g., where the gene is aberrantly expressed, and the therapeutic agent regulates the aberrant expression by restoring its normal expression pattern.

Arrays

The present invention also relates to an ordered array of polynucleotide probes and specific-binding partners (e.g., antibodies) for detecting the expression of differentially regulated cancer genes in a sample, comprising, one or more polynucleotide probes or specific binding partners associated with a solid support or in separate receptacles, wherein each probe is specific for differentially regulated cancer genes, and the probes comprise a nucleotide sequence of SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 which is specific for said gene, a nucleotide sequence having sequence identity to SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and/or 99 which is specific for said gene or polynucleotide, or complements thereto, or a specific-binding partner which is specific for differentially regulated cancer genes.

The phrase “ordered array” indicates that the probes are arranged in an identifiable or position-addressable pattern, e.g., such as the arrays disclosed in U.S. Pat. Nos. 6,156,501, 6,077,673, 6,054,270, 5,723,320, 5,700,637, WO09919711, WO00023803. The probes are associated with the solid support in any effective way. For instance, the probes can be bound to the solid support, either by polymerizing the probes on the substrate, or by attaching a probe to the substrate. Association can be, covalent, electrostatic, noncovalent, hydrophobic, hydrophilic, noncovalent, coordination, adsorbed, absorbed, polar, etc. When fibers or hollow filaments are utilized for the array, the probes can fill the hollow orifice, be absorbed into the solid filament, be attached to the surface of the orifice, etc. Probes can be of any effective size, sequence identity, composition, etc., as already discussed.

Transgenic Animals

The present invention also relates to transgenic animals comprising differentially regulated cancer genes genes, and homologs thereof (Methods of maling transgenic animals, and associated recombinant technology, can be accomplished conventionally, e.g., as described in Transgenic Animal Technology, Pinkert et al., 2^{nd} Edition, Academic Press, 2002.) Such genes, as discussed in more detail below, include, but are not limited to, functionally-disrupted genes, mutated genes, ectopically or selectively-expressed genes, inducible or regulatable genes, etc. These transgenic animals can be produced according to any suitable technique or method, including homologous recombination, mutagenesis (e.g., ENU, Rathkolb et al., Exp. Physiol., 85(6):635-644, 2000), and the tetracycline-regulated gene expression system (e.g., U.S. Pat No. 6,242,667). The term “gene” as used herein includes any part of a gene, i.e., regulatory sequences, promoters, enhancers, exons, introns, coding sequences, etc. The differentially regulated cancer genes nucleic acid present in the construct or transgene can be naturally-occurring wild-type, polymorphic, or mutated. Where the animal is a non-human animal, its homolog can be used instead. Transgenic animals can be susceptible to cancer, e.g., prostate or breast cancer.

Along these lines, polynucleotides of the present invention can be used to create transgenic animals, e.g. a non-human animal, comprising at least one cell whose genome comprises a functional disruption of a differentially regulated cancer gene, or a homolog thereof (e.g., a mouse homolog when a mouse is used). By the phrases “functional disruption” or “functionally disrupted,” it is meant that the gene does not express a biologically-active product. It can be substantially deficient in at least one functional activity coded for by the gene. Expression of a polypeptide can be substantially absent, i.e., essentially undetectable amounts are made. However, polypeptide can also be made, but which is deficient in activity, e.g., where only an amino-terminal portion of the gene product is produced.

The transgenic animal can comprise one or more cells. When substantially all its cells contain the engineered gene, it can be referred to as a transgenic animal “whose genome comprises” the engineered gene. This indicates that the endogenous gene loci of the animal has been modified and substantially all cells contain such modification.

Functional disruption of the gene can be accomplished in any effective way, including, e.g., introduction of a stop codon into any part of the coding sequence such that the resulting polypeptide is biologically inactive (e.g., because it lacks a catalytic domain, a ligand binding domain, etc.), introduction of a mutation into a promoter or other regulatory sequence that is effective to turn it off, or reduce transcription of the gene, insertion of an exogenous sequence into the gene which inactivates it (e.g., which disrupts the production of a biologically-active polypeptide or which disrupts the promoter or other transcriptional machinery), deletion of sequences from the differentially regulated cancer genes gene (or homolog thereof), etc. Examples of transgenic animals having functionally disrupted genes are well known, e.g., as described in U.S. Pat. Nos. 6,239,326, 6,225,525, 6,207,878, 6,194,633, 6,187,992, 6,180,849, 6,177,610, 6,100,445, 6,087,555, 6,080,910, 6,069,297, 6,060,642, 6,028,244, 6,013,858, 5,981,830, 5,866,760, 5,859,314, 5,850,004, 5,817,912, 5,789,654, 5,777,195, and 5,569,824. A transgenic animal which comprises the functional disruption can also be referred to as a “knock-out” animal, since the biological activity of its differentially regulated cancer genes genes has been “knocked-out” Knock-outs can be homozygous or heterozygous.

For creating functionally disrupted genes, and other gene mutations, homologous recombination technology is of special interest since it allows specific regions of the genome to be targeted. Using homologous recombination methods, genes can be specifically-inactivated, specific mutations can be introduced, and exogenous sequences can be introduced at specific sites. These methods are well known in the art, e.g., as described in the patents above. See, also, Robertson, Biol. Reproduc., 44(2):238-245, 1991. Generally, the genetic engineering is performed in an embryonic stem (ES) cell, or other pluripotent cell line (e.g., adult stem cells, EG cells), and that genetically-modified cell (or nucleus) is used to create a whole organism. Nuclear transfer can be used in combination with homologous recombination technologies.

For example, the differentially regulated cancer genes locus can be disrupted in mouse ES cells using a positive-negative selection method (e.g., Mansour et al., Nature, 336:348-352, 1988). In this method, a targeting vector can be constructed which comprises a part of the gene to be targeted. A selectable marker, such as neomycin resistance genes, can be inserted into a differentially regulated cancer genes exon present in the targeting vector, disrupting it. When the vector recombines with the ES cell genome, it disrupts the function of the gene. The presence in the cell of the vector can be determined by expression of neomycin resistance. See, e.g., U.S. Pat. No. 6,239,326. Cells having at least one functionally disrupted gene can be used to make chimeric and germline animals, e.g., animals having somatic and/or germ cells comprising the engineered gene. Homozygous knock-out animals can be obtained from breeding heterozygous knock-out animals. See, e.g., U.S. Pat. No. 6,225,525.

The present invention also relates to non-human, transgenic animal whose genome comprises recombinant differentially regulated cancer nucleic acid (and homologs thereof) operatively linked to an expression control sequence effective to express said coding sequence, e.g., in prostate and/or breast tissues. Such a transgenic animal can also be referred to as a “knock-in” animal since an exogenous gene has been introduced, stably, into its genome.

A recombinant differentially regulated cancer genes nucleic acid refers to a polynucleotide which has been introduced into a target host cell and optionally modified, such as cells derived from animals, plants, bacteria, yeast, etc. A recombinant differentially regulated cancer genes includes completely synthetic nucleic acid sequences, semi-synthetic nucleic acid sequences, sequences derived from natural sources, and chimeras thereof. “Operable linkage” has the meaning used through the specification, i.e., placed in a functional relationship with another nucleic acid. When a gene is operably linked to an expression control sequence, as explained above, it indicates that the gene (e.g., coding sequence) is joined to the expression control sequence (e.g., promoter) in such a way that facilitates transcription and translation of the coding sequence. As described above, the phrase “genome” indicates that the genome of the cell has been modified. In this case, the recombinant differentially regulated cancer genes has been stably integrated into the genome of the animal. The differentially regulated cancer genes nucleic acid (e.g., a coding sequence) in operable linkage with the expression control sequence can also be referred to as a construct or transgene.

The present invention also relates to a transgenic animal which contains a functionally disrupted and a transgene stably integrated into the animals genome. Such an animal can be constructed using combinations any of the above- and below-mentioned methods. Such animals have any of the aforementioned uses, including permitting the knock-out of the normal gene and its replacement with a mutated gene. Such a transgene can be integrated at the endogenous gene locus so that the functional disruption and “knock-in” are carried out in the same step.

In addition to the methods mentioned above, transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115l-4124, 1993; Games et al. Nature, 373:523-527, 1995; Valancius and Smithies, Mol. Cell. Bio., 11:1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617, 1993; Cibelli et al., Science, 280:1256-1258, 1998. For guidance on recombinase excision systems, see, e.g., U.S. Pat. Nos. 5,626,159, 5,527,695, and 5,434,066. See also, Orban, P.C., et al., “Tissue- and Site-Specific DNA Recombination in Transgenic Mice,” Proc. Natl. Acad. Sci. USA, 89:6861-6865 (1992); O'Gorman, S., et al., “Recombinase-Mediated Gene Activation and Site-Specific Integration in Mammalian Cells,” Science, 251:1351-1355 (1991); Sauer, B., et al., “Cre-stimulated recombination at loxP-Containing DNA sequences placed into the mammalian genome,” Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest 100:169-179; Barlow, C. et al. (1997) Nucl. Acids Res. 25:2543-2545; Araki, K. et al. (1997) Nucl. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

A polynucleotide according to the present invention can be introduced into any non-human animal, including a non-human mammal, mouse (Hogan et al., Manipulating the Mouse Embryo: A Laboratory Manual Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePamphilis et al., BioTechniques, 6:662-680, 1988. Transgenic animals can be produced by the methods described in U.S. Pat. No. 5,994,618, and utilized for any of the utilities described therein.

Database

The present invention also relates to electronic forms of polynucleotides, polypeptides, etc., of the present invention, including computer-readable medium (e.g., magnetic, optical, etc., stored in any suitable format, such as flat files or hierarchical files) which comprise such sequences, or fragments thereof, e-commerce-related means, etc. Along these lines, the present invention relates to methods of retrieving gene sequences from a computer-readable medium, comprising, one or more of the following steps in any effective order, e.g., selecting a cell or gene expression profile, e.g., a profile that specifies that said gene is differentially expressed in breast and/or prostate cancer, and retrieving said differentially expressed gene sequences, where the gene sequences consist of the genes represented by SEQ ID NOS 1, 3, 5, 7, 9, 11, 13, 15, 17, 19, 21,.23, 25, 27, 29, 45, 47, 50, 48, 52, 54, 56, 58, 60, 62, 64, 66, 68, 70, 72, 74, 84, 85, 86, 88, 95, and/or 99.

A “gene expression profile” means the list of tissues, cells, etc., in which a defined gene is expressed (i.e, transcribed and/or translated). A “cell expression profile” means the genes which are expressed in the particular cell type. The profile can be a list of the tissues in which the gene is expressed, but can include additional information as well, including level of expression (e.g., a quantity as compared or normalized to a control gene), and information on temporal (e.g., at what point in the cell-cycle or developmental program) and spatial expression. By the phrase “selecting a gene or cell expression profile,” it is meant that a user decides what type oQf gene or cell expression pattern he is interested in retrieving. Any pattern of expression preferences may be selected. The selecting can be performed by any effective method. In general, “selecting” refers to the process in which a user forms a query that is used to search a database of gene expression profiles. The step of retrieving involves searching for results in a database that correspond to the query set forth in the selecting step. Any suitable algorithm can be utilized to perform the search query, including algorithms that look for matches, or that perform optimization between query and data The database is information that has been stored in an appropriate storage medium, having a suitable computer-readable format. Once results are retrieved, they can be displayed in any suitable format, such as HTML.

For instance, the user may be interested in identifying genes that are differentially expressed in cancer. He may not care whether small amounts of expression occur in other tissues, as long as such genes are not expressed in peripheral blood lymphocytes. A query is formed by the user to retrieve the set of genes from the database having the desired gene or cell expression profile. Once the query is inputted into the system, a search algorithm is used to interrogate the database, and retrieve results.

Advertising, Licensing, etc., Methods

The present invention also relates to methods of advertising, licensing, selling, purchasing, brokering, etc., genes, polynucleotides, specific-binding partners, antibodies, etc., of the present invention. Methods can comprises, e.g., displaying a differentially regulated cancer genes gene, differentially regulated cancer genes polypeptide, or antibody specific for differentially regulated cancer genes in a printed or computer-readable medium (e.g., on the Web or Internet), accepting an offer to purchase said gene, polypeptide, or antibody.

Other

A polynucleotide, probe, polypeptide, antibody, specific-binding partner, etc., according to the present invention can be isolated. The term “isolated” means that the material is in a form in which it is not found in its original environment or in nature, e.g., more concentrated, more purified, separated from component, etc. An isolated polynucleotide includes, e.g., a polynucleotide having the sequenced separated from the chromosomal DNA found in a living animal, e.g., as the complete gene, a transcript, or a cDNA. This polynucleotide can be part of a vector or inserted into a chromosome (by specific gene-targeting or by random integration at a position other than its normal position) and still be isolated in that it is not in a form that is found in its natural environment. A polynucleotide, polypeptide, etc., of the present invention can also be substantially purified. By substantially purified, it is meant that polynucleotide or polypeptide is separated and is essentially free from other polynucleotides or polypeptides, i.e., the polynucleotide or polypeptide is the primary and active constituent. A polynucleotide can also be a recombinant molecule. By “recombinant,” it is meant that the polynucleotide is an arrangement or form which does not occur in nature. For instance, a recombinant molecule comprising a promoter sequence would not encompass the naturally-occurring gene, but would include the promoter operably linked to a coding sequence not associated with it in nature, e.g., a reporter gene, or a truncation of the normal coding sequence.

The term “marker” is used herein to indicate a means for detecting or labeling a target. A marker can be a polynucleotide (usually referred to as a “probe”), polypeptide (e.g., an antibody conjugated to a detectable label), PNA, or any effective material.

The topic headings set forth above are meant as guidance where certain information can be found in the application, but are not intended to be the only source in the application where information on such topic can be found. Reference materials For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. The entire disclosure of all applications, patents and publications, cited above and in the figures are hereby incorporated by reference in their entirety.

TABLE 1

| Clone ID | Protein-L* | Location | Domains Names |
|---|---|---|---|
| 1. Pcp0749-2z | 1564aa | Nuclear | 1. ZnF_C3H1 domain: 36-63aa; |
| | | | 2. Caldesmon domain: 423-1027aa; |
| | | | 3. Coiled coil: 162-197aa; |
| | | | 4. Coiled coil: 645-789aa; |
| | | | 5. Coiled coil: 1339-1366aa. |
| 2. Pcp0389Az | 694aa | Extracellular | 1. Coiled coil: 17-51aa; |
| | | | 2. Methyl-accepting chemotaxis-like domain |
| | | | (MA): 101-200aa. |
| 3. Pcp0814z | 279aa | Membrane | 1. Transmembrane domain: 83-105aa; |
| | | | 2. Transmembrane domain: 120-142aa; |
| | | | 3. Transmembrane domain: 192-211aa; |
| | | | 4. MotA/TolQ/ExbB proton channel domain: 34-156aa; |
| | | | 5. Coiled coil: 226-253aa. |
| 4. Pcp0623 | 2697aa | Nuclear | 1. Caldesmon domain: 590-884aa. |
| 5. Pcp0815 | 1041aa | Nuclear | 1. ZF_C2H2: 371-393aa; |
| | | | 2. ZF_C2H2: 399-421aa; |
| | | | 3. ZF_C2H2: 621-651aa; |
| | | | 4. ZF_C2H2: 657-679aa; |
| | | | 5. ZF_C2H2: 689-711aa; |
| | | | 6. ZF_C2H2: 909-931aa; |
| | | | 7. ZF_C2H2: 938-961aa; |
| | | | 8. PP_M1 Phosphoprotein domain: 754-923aa. |
| 6. Pcp0840z | 243aa | Nuclear | 1. ZnF_C2H2 domains: 12-37aa; |
| | | | 2. ZnF_C2H2 domains: 173-198aa; |
| | | | 3. ZnF_C2H2 domains: 208-230aa. |
| 7. Pcp0424Az | 789aa | Cytoplasm | 1. Dynamin, large GTPase domain: 54-308aa. (GTP- |
| | | | binding); |
| | | | 2. Dynamin GTPase effector domain: 692-783aa. |
| 7. Pcp0424Bz | 763aa | Cytoplasm | 1. Dynamin, large GTPase domain: 54-308aa. (GTP- |
| | | | binding); |
| | | | 2. Dynamin GTPase effector domain: 692783aa; |
| 7. Pcp0424Cz | 752aa | Cytoplasm | 1. Dynamin, large GTPase domain: 54-308aa. (GTP- |
| | | | binding); |
| | | | 2. Dynamin GTPase effector domain: 692-783aa. |
| 8. Pc0382 | 1584aa | Nuclear | 1. SAM domain: 11-78aa; |
| | | | 2. Kinesin domain: 1079-1103aa. |
| 9. Pcp0816 | 1013aa | Membrane | 1. Signal peptide: 1-38aa; |
| | | | 2. EGF-like domain: 274-308aa; |
| | | | 3. Transmembrane domain: 908-930aa. |
| 10. Pcp0480 | 171aa | Nuclear | 1. Estrogen receptor: 1-169aa. |
| 11. Pcp0842x | 2221aa | Cytoplasm | 1. SET7 domain: 630-842aa |

L* stands for protein length in amino acids

TABLE 2

| Clone ID | Locus | Associated diseases |
|---|---|---|
| 1. Pcp0749-2z | 13q14.11 | 1. Rieger syndrome type 2 at 13q14; |
| | | 2. Low grade B-cell malignancy at 13q14. |
| 2. Pcp0389Az | 6q22.33 | 1. IgA Nephropathy at 6q22-q23; |
| | | 2. Autosomal recessive craniometaphyseal dysplasia at 6q21-q22; |
| | | 3. Heterocellular hereditary persistence of fetal hemoglobin at 6q22.3-q23.1; |
| | | 4. Oculodentodigital dysplasia at 6q22-q24; |
| | | 5. Susceptibility to severe hepatic fibrosis due to Schistosoma mansoni |
| | | infection at 6q22-q23. |
| 3. Pcp0814z | 12p13.3 | 1. Chromosomal abnormalities associated with breast and |
| | | ovary cancer. |
| 4. Pcp0623 | 5p13.2 | |
| 5. Pcp0815 | 14q11.1 | 1. Respiratory allergies (asthma) at 14q11.1 |
| 6. Pcp0840z | 7p15.1 | |
| 7. Pcp0424Az | 12p12.3 | 1. Alzheimer disease familial type 5 at 12p11.23-q13.12; |
| | | 2. Fibrosis of extraocular muscles congenital 1at 12p11.2-q12; |
| | | 3. Hypertension with brachydactyly at 12p12.2-p11.2. |
| 7. Pcp0424Bz | 12p12.3 | same as Bcu0424Az. |
| 7. Pcp0424Cz | 12p12.3 | same as Bcu0424Az. |
| 8. Pc0382 | 7q21.3 | 1. Split-hand/foot malformation type-1(SHFM1) at 7q21.2-q21.3; |
| | | 2. SHFM with sensorineural hearing loss (SHFM1D at 7q21.2-21.3.); |
| | | 4. Malignant hyperthermia susceptibility 3 at 7q21-q22; |
| | | 5. Myoclonic dystonia-11 at 7q21. |
| 9. Pcp0816 | 1p13.1 | 1. Vesicoureteral reflux (VUR) at 1p13; |
| | | 2. Trisomy and Monosomy at 1p13 cause cancers in prostate, ovary |
| | | and breast. |
| 10. Pcp0480 | 6q25.1 | 1. Estrogen receptor-1 at 6q25.1(Alternative isoforms are related to |
| | | breast cancer); |
| | | 2. Schizophrenia-5 at 6q26-q13; |
| | | 3. Insulin-dependent diabetes mellitus-8 at 6q25-q27. |
| 11. Pcp0842x | 6q23.3 | 1. Dilated cardiomyopathy 1J (CMD1J) at 6q23-q24; |
| | | 2. Dilated cardiomyopathy 1F (CMD1F) at 6q23; |
| | | 3. Oculodentodigital dysplasia (ODDD) at 6q22-q24; |
| | | 4. Susceptibility to severe hepatic fibrosis due to Schistosoma mansoni |
| | | infection at 6q22-q23; |
| | | 5. IgA nephropathy at 6q22-q23. |

TABLE 3

| Gene | | | |
|---|---|---|---|
| Name | SEQ ID NO | Expression | |
| PCP0749 | 1-4 | UP | |
| PCP0389 | 5-8 | UP | |
| PCP0814 | 9-10 | DOWN | |
| PCP0623 | 11-12 | DOWN | |
| PCP0815 | 13-14 | UP | |
| PCP0840 | 15-16 | DOWN | |
| PCP0424 | 17-18 (A); 19-20 (B); | UP | |
| | 21-22 (C) | | |
| PC0382 | 23-24 | UP | |
| PCP0816 | 25-26 | UP | |
| PCP0480 | 27-28 | UP | normal expression |
| | | | restricted to muscle |
| | | | and uterus |
| PCP0842 | 29-30 | UP | |

TABLE 4

| GENE | EXPRESSION | LENGTH | DOMAINS |
|---|---|---|---|
| 1. PCP0405 | DOWN | 1379 | 1. CUB domain: 93-209aa; |
| (SEQ ID NO 45-46) | | | 2. DSL domain: 222-280aa; |
| | | | 3. Kelch domain: 480-531aa; |
| | | | 4. Kelch domain: 532-591aa; |
| | | | 5. PSI domain: 614-657aa; |
| | | | 6. PSI domain: 666-709aa; |
| | | | 7. CLECT domain: 748-873aa; |
| | | | 8. PSI domain: 889-939aa; |
| | | | 9. PSI domain: 942-1012aa; |
| | | | 10. EGF-like domain: 1014-1057aa; |
| | | | 11. EGF-like domain: 1060-1106aa; |
| | | | 12. Transmembrane domain: 1230-1252aa. |
| 2. PCP0454A | DOWN | 3863 | 1. Internal repeat 2: 19-72; |
| (SEQ ID NO 50-51) | | | 2. Internal repeat 1: 71-135; |
| | | | 3. Internal repeat 2: 332-385; |
| | | | 4. Internal repeat 1: 488-554. |
| PCP0454B | DOWN | 577 | 1. Internal repeat 1: 1-137; |
| (SEQ ID NO 48-49) | | | 2. AAA domain: 241-408. |
| 3. PCP0459 | UP | 715 | 1. Gag p10 domain: 1-89; |
| (SEQ ID NO 52-53) | | | 2. Gag p24 domain: 360-573; |
| | | | 3. ZnF C2HC domain: 592-608; |
| | | | 4. ZnF C2HC domain: 629-645. |
| 4. PC0177A | UP | 1744 | 1. Coiled coil: 646-685; |
| (SEQ ID NO 54-55) | | | 2. Coiled coil: 1469-1481; |
| | | | 3. Coiled coil: 1656-1684. |
| PC0177B | UP | 1709 | 1. Coiled coil: 611-650; |
| (SEQ ID NO 56-57) | | | 2. Coiled coil: 1434-1456; |
| | | | 3. Coiled coil: 1621-1649. |
| PC0177C | UP | 1908 | 1. Coiled coil: 611-650; |
| (SEQ ID NO 58-59) | | | 2. Coiled coil: 1434-1456; |
| | | | 3. Coiled coil: 1621-1649. |
| PC0177D | UP | 1309 | 1. Coiled coil: 611-650 |
| (SEQ ID NO 60-61) | | | |
| 5. PCP0557 | UP | 1593 | 1. HisKA: 565-620; |
| (SEQ ID NO 62-63) | | | 2. Coiled coil: 933-965; |
| | | | 3. Coiled coil: 1464-1491. |
| 6. PCP0664 | UP | 112 | 1. Transmembrane: 4-26. |
| (SEQ ID NO 64-65) | | | |
| 7. PCP0677 | UP | 89 | No domain found. |
| (SEQ ID NO 66-67) | | | |
| 8. PCP0762 | UP | 221 | 1. SCAN domain 42-137 |
| (SEQ ID NO 68-69) | | | |
| 9. PCP0806 | UP | 548 | 1. SCOP domain: 10-122 |
| (SEQ ID NO 70-71) | | | 2. Coiled coil: 374-409 |
| 10. PCP0815A | UP | 1005 | 1. ZnF C2H2 domain: 371-393; |
| (SEQ ID NO 72-73) | | | 2. ZnF C2H2 domain: 399-421; |
| | | | 3. ZnF C2H2 domain: 629-651; |
| | | | 4. ZnF C2H2 domain: 657-679; |
| | | | 5. ZnF C2H2 domain: 689-711; |
| | | | 6. ZnF C2H2 domain: 909-931; |
| | | | 7. ZnF C2H2 domain: 9380961. |
| PCP0815C | UP | 198 | No domain found |
| (SEQ ID NO 74-75) | | | |

TABLE 5

| Clone ID | Locus | Diseases |
|---|---|---|
| PCP0405 | 10q26 | Cancers |
| PCP0454 | 6q15 | Amaurosis Congenita Of Leber V; |
| | | Cardiomyopathy, Dilated, 1k (Cmd1k); |
| | | Chorioretinal Atrophy, Progressive Bifocal; |
| | | Macular Dystrophy, Retinal, 1, North Carolina Type (Mcdr1) |
| PCP0459 | 22q11.21 | Cancers |
| PC0177 | 10p11.22 | Diabetes |
| PCP0577 | Xq25-q26.3 | Mental Retardation, X-Linked, With Short Stature, Small Testes, |
| | | Muscle Wasting, And Tremor; |
| | | Hypertrichosis, Congenital Generalized (Htc2); |
| | | Borjeson-Forssman-Lehmann Syndrome (Bfls); |
| | | Mental Retardation |
| PCP0664 | Xq25-Xq26 | Mental Retardation, X-Linked, With Isolated Growth Hormone |
| | | Deficiency (Mrgh) |
| | | Hypertrichosis, Congenital Generalized (Htc2); |
| | | Mental Retardation, X-Linked, South African Type; |
| | | Borjeson-Forssman-Lehmann Syndrome (Bfls); |
| | | Mental Retardation With Optic Atrophy, Deafness, And Seizures; |
| | | Mental Retardation |
| PCP0677 | 12q15 | Scapuloperoneal Myopathy (SPM); |
| | | Cancers (e.g., glioma) |
| PCP0762 | 18q12.1 | Cancers |
| PCP0806 | 2q37.3 | Gracile Syndrome; |
| | | Holoprosencephaly 6 |
| PCP0815 | 14q11.1-q12 | Arrhythmogenic Right Ventricular Dysplasia, Familial, 3 (Arvd3); |
| | | Radiation Sensitivity/Chromosome Instability Syndrome, |
| | | Autosomal Dominant; |
| | | Asthma |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding International application No. PCT/US03/01943, filed Jan. 24, 2003; U.S. application Ser. No. 10/054,935, filed Jan. 25, 2002; U.S. Provisional Application Ser. No. 60/356,130, filed Feb. 14, 2002; U.S. application Ser. No. 10/102,946, filed Mar. 22, 2002; U.S. application Ser. No. 10/117,229, filed Apr. 8, 2002; U.S. application Ser. No. 10/144,198, filed May 14, 2002 and U.S. application Ser. No. 10/197,824, filed Jul. 19, 2002, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. An isolated polynucleotide comprising,
a polynucleotide sequence which codes without interruption for the amino acid sequence selected from SEQ ID NOS 2, 4, 6, 8, 10, 12, 14, 16, 18, 20, 22, 24, 26, 28, 30, 46, 51, 49, 53, 55, 57, 59, 61, 63, 65, 67, 69, 71, 73, 75, 87, 89, 96, and 100, or a complement thereto.

2. An isolated polynucleotide comprising,
a polynucleotide sequence having 95% or more sequence identity along the entire length of a polynucleotide sequence selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and 99, or a complement thereto.

3. An isolated polynucleotide comprising,
a differentially regulated mammalian cancer gene having a polynucleotide sequence which hybridizes under high stringency conditions to a polynucleotide having a polynucleotide sequence set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and 99, or a complement thereto

4. An isolated polynucleotide of claim 3, wherein said high stringency conditions comprise hybridizing 42° C. in 5×SSPE, 0.3% SDS, and 50% formamide, and washes at 65° C. for 15 minutes in 2×SSC, and 0.2% SDS.

5. An isolated human polypeptide which is differentially regulated in a human cancer, comprising the complete amino acid sequence as set forth in SEQ ID NOS 2, 4, 6, 8, 10, 12, 14, 16, 18, 20, 22, 24, 26, 28, 30, 46, 51, 49, 53, 55, 57, 59, 61, 63, 65, 67, 69, 71, 73, 75, 87, 89, 96, or 100, or a specific fragment thereof.

6. An isolated mammalian polypeptide which is differentially regulated in a mammalian cancer, having 95% or more sequence identity along its entire length to a complete amino acid sequence as set forth in SEQ ID NOS 2, 4, 6, 8, 10, 12, 14, 16, 18, 20, 22, 24, 26, 28, 30, 46, 51, 49, 53, 55, 57, 59, 61, 63, 65, 67, 69, 71, 73, 75, 87, 89, 96, or 100, or a specific fragment thereof.

7. An isolated polypeptide which is coded for by a polynucleotide of claim 2.

8. A method of detecting a nucleic acid coding for human differentially regulated cancer genes, comprising,
contacting a sample comprising nucleic acid with a polynucleotide probe specific for a differentially regulated human cancer gene of claim 1 under conditions effective for said probe to hybridize specifically with said gene, and
detecting hybridization between said probe and said nucleic acid.

9. A method of claim 8, wherein said detecting is performed by:
Northern blot analysis, polymerase chain reaction (PCR), reverse taanscriptase PCR, RACE PCR, or in situ hybridization.

10. A method of treating a cancer showing altered expression of differentially regulated human cancer gene, comprising:
administering to a subject in need thereof a therapeutic agent which is effective for regulating expression of a differentially regulated human cancer gene of claim 1

11. A method of claim 10, wherein said agent is an antibody or an antisense which is effective to inhibit translation of said gene.

12. A method of diagnosing a cancer associated with abnormal expression of a differentially regulated human cancer gene, or determining a subject's susceptibility to such cancer, comprising:
assessing the expression of a differentially regulated human cancer gene of claim 1 in a tissue sample comprising prostate or breast cells.

13. A method of claim 12, wherein assessing is:
measuring expression levels of said gene, determining the genomic structure of said gene, determining the mRNA structure of transcripts from said gene, or measuring the expression levels of polypeptide coded for by said gene.

14. A method of claim 12, wherein said assessing is performed by:
Northern blot analysis, polymerase chain reaction (PCR), reverse transcriptase PCR, RACE PCR, or in situ hybridization, and
using a polynucleotide probe having a polynucleotide sequence selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and 99, or a complement thereto.

15. A method of assessing a therapeutic or preventative intervention in a subject having cancer, comprising,
determining the expression levels of differentially regulated cancer genes of claim 1 in a tissue sample comprising prostate or breast cells.

16. A method for identifying an agent that modulates the expression of differentially regulated cancer gene expressed in breast or prostate tissue cells, comprising,
contacting a cell population comprising breast or prostate cells with a test agent under conditions effective for said test agent to modulate the expression of a differentially regulated cancer gene of claim 1 in said breast or prostate cells, and
determining whether said test agent modulates said differentially regulated cancer gene.

17. A method for identifying an agent that modulates the expression of a polypeptide coded for by a differentially regulated cancer gene expressed in breast or prostate tissue cells, comprising,
contacting a polypeptide coded for by a differentially regulated cancer gene of claim 1, with a test agent under conditions effective for said test agent to modulate said polypeptide, and
determining whether said test agent modulates said polypeptide.

18. A method of claim 16, wherein said test agent is an antibody.

19. A method of detecting polymorphisms in differentially regulated cancer genes comprising:
comparing the structure of: genomic DNA comprising all or part of differentially regulated cancer genes, mRNA comprising all or part of differentially regulated cancer genes, cDNA comprising all or part of differentially regulated cancer genes, or a polypeptide comprising all or part of differentially regulated cancer genes, with the structure of a differentially regulated cancer gene as set forth in a polynucleotide sequence selected from SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, and 99.

20. A method of identifying a genetic basis for a prostate or breast cancer, or susceptibility thereto, comprising:
determining the association of cancer, suceptibility thereto, with a polynucleotide of claim 1.

21. A method of claim 20, wherein determining is performed by producing a human-linkage map using said polynucleotide.

22. A method of claim 20, wherein determining is performed by comparing the nucleotide sequences of said polynucleotide between normal subjects and subjects having cancer.

23. A non-human, transgenic mammal, or a cell thereof, whose genome comprises a functional disruption of a homolog of a differentially regulated cancer gene of claim 1.

24. An antibody which is specific-for a differentially regulated cancer gene polypeptide of claim 5.

25. A method of advertising differentially regulated cancer genes for sale, commercial use, or licensing, comprising,
displaying in a computer-readable medium a polynucleotide set forth in SEQ ID NOS 1, 3, 5, 7, 9, 11, 13, 15, 17, 19, 21, 23, 25, 27, 29, 45, 47, 50, 48, 52, 54, 56, 58, 60, 62, 64, 66, 68, 70, 72, 74, 84, 85, 86, 88, 95, or 99 of claim 1, effective specific fragments thereof, or complements thereto.

26. A method of selecting a polynucleotide sequence coding for differentially regulated cancer genes from a database comprising polynucleotide sequences, comprising
displaying, in a computer-readable medium, a polynucleotide sequence or polypeptide sequence for one or more differentially regulated cancer genes of claim 1, or complements to the polynucleotides sequence,
wherein said displayed sequences have been retrieved from said database upon selection by a user.
